(19) United States
(12) Patent Application Publication
(10) Pub. No.: US 2026/0317401 A1
(43) Pub. Date: Oct. 15, 2026

(54) PREDICTIVE MODELING OF DISSOLVED OXYGEN EXCURSIONS FOR REGULATING BIOREACTOR

(71) Applicant: Regeneron Pharmaceuticals, Inc., Tarrytown, NY (US)

(72) Inventors: Katherine Resch, Tarrytown, NY (US); Christopher Sevinsky, Tarrytown, NY (US); Patrick Faughnan, Tarrytown, NY (US); Paul Steve, Tarrytown, NY (US)

(21) Appl. No.: 19/568,337

(22) Filed: Mar. 16, 2026

Related U.S. Application Data
(60) Provisional application No. 63/773,229, filed on Mar. 17, 2025.
(60) Provisional application No. 64/006,108, filed on Mar. 15, 2026.

(51) Int. Cl. C07K 16/00; C12N 5/071; C12P 21/00; C12Q 1/48; C12Q 1/6851; C12Q 1/686; C12Q 1/6869; G06N 20/00; G16B 25/10; G16B 40/20

(52) U.S. Cl. CPC C07K 16/00; C12N 5/0682; C12N 5/0686; C12P 21/00; C12Q 1/48; C12Q 1/6851; C12Q 1/686; C12Q 1/6869; C12Y 207/07; G06N 20/00; G16B 25/10; G16B 40/20; C07K 2317/14; C07K 2319/00; C12N 2500/02; C12N 2510/04

(57) ABSTRACT

The present invention generally pertains to methods of cultivating cells and producing a recombinant protein of interest. In particular, the present invention pertains to determining a cell culture process after a dissolved oxygen excursion based on correlating the dissolved oxygen excursion or expression of genetic biomarkers to predicted product quality.

### CROSS-REFENCE TO RELATED APPLICATIONS

This application claims the benefit of U.S. Provisional Patent Application No. 64/006,108, filed on Mar. 15, 2026 and U.S. Provisional Patent Application No. 63/773,229, filed on Mar. 17, 2025 each of which is incorporated herein by reference in its entirety.

### BACKGROUND

Maintaining process control during commercial antibody manufacturing is important for ensuring safe, high-quality medicines for patients. Excursions to acceptable manufacturing conditions, for example, shifts in dissolved oxygen (DO) concentration, occur during routine operation, which may adversely impact cell growth and protein yield. However, the link between measured excursions and product quality is understudied. Frequently observed DO excursions during commercial production place a significant burden on the quality system, requiring investigation time to resolve prior to lot disposition.

Using conventional methods, a cell culture batch that undergoes excessive DO excursions must be cultivated to its harvest day and the protein must then be harvested, purified, and evaluated before it can be determined that the earlier DO excursions resulted in a protein product that fails to meet a performance target. Thus, a need exists for methods for proactively predicting an effect of DO excursions on product quality, allowing for determining a modified manufacturing process to accommodate the excursion, or the preemptive termination of a cell culture batch without having to subject the batch to costly quality evaluation.

### SUMMARY

Methods have been developed for predicting an effect of DO excursions on recombinant protein product quality, therefore allowing for determining the outcome of a cell culture batch without expensive quality testing, and further allowing for modifying a cell culture process in order to ensure meeting performance targets even after process excursions. Laboratory-scale design of experiments were conducted to assess the impact of transient DO excursions on process performance and product quality, using monoclonal antibodies as an example, and models were produced that successfully linked the properties of DO excursions to the resulting outcome on cell culture performance and product quality. Results showed that titer is affected by many or all input parameters. Product quality is directly influenced by the magnitude and duration of DO excursions, with only extreme excursions causing specification failures and moderate conditions yielding acceptable antibodies. Applying the disclosures disclosed herein provides methods for generating models to predict the effects of excursions and methods for adjusting cell culture conditions to mitigate the detrimental effects of the excursions on titer and product quality.

Additionally, methods have been developed for predicting cell culture performance based on gene expression analysis as early as day 4 of a production process. A transcriptomic analysis of recombinant cells under various cell culture conditions was performed, and models were produced that successfully predicted viable cell density, titer, product quality attributes, and final product quality based on differentially expressed genes. This method also allowed for the identification of genetic biomarkers with particular predictive importance for cell culture process outcomes. Applying the disclosures disclosed herein provides methods for using biomarkers to reliably predict outcomes of a cell culture process, even at an early stage.

Further, methods have been developed for predicting product quality outcomes based on online measurements of cell culture health, such as can be acquired using a blood gas analyzer. Parameters related to cell culture health, such as pH, temperature, gas content, and metabolites, were measured throughout a production process, and the measured data was used to generate multivariate models capable of reliably predicting product quality attributes at the end of the production process.

This disclosure provides methods for controlling the duration of a cell culture batch. In some exemplary aspects, the methods can comprise (a) culturing cells in a cell culture, wherein said cells produce a recombinant protein and said cell culture undergoes at least one dissolved oxygen (DO) excursion; (b) correlating said at least one DO excursion to at least one predicted product quality using at least one regression model; and (c) controlling the duration of said cell culture batch based on said at least one predicted product quality.

In one aspect, said controlling further comprises comparing each of said at least one predicted product qualities to a performance target for said product quality. In a specific aspect, said controlling further comprises terminating said cell culture batch when said at least one predicted product quality fails to meet said performance target. In another specific aspect, said at least one product quality comprises protein titer, said predicted protein titer is lower than a performance target for protein titer, and said controlling further comprises increasing a duration of said cell culture batch.

In one aspect, said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one product quality is a product quality attribute. In still another aspect, said at least one product quality is a critical quality attribute.

In one aspect, said at least one product quality comprises protein titer and a performance target for protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.

In one aspect, said at least one product quality comprises protein purity and a performance target for protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%. In a specific aspect, said protein purity is measured by liquid chromatography, capillary electrophoresis, gel electrophoresis, or mass spectrometry. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is 1D or 2D sodium dodecyl sulfate-polyacrylamide gel electrophoresis.

In one aspect, said at least one product quality comprises high molecular weight species and a performance target for high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said high molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE). In a different specific aspect, said high molecular weight species are measured by size exclusion chromatography, native polyacrylamide gel electrophoresis, capillary electrophoresis, light scattering, dynamic light scattering, analytical ultracentrifugation, scanning electron microscopy, transmission electron microscopy, atomic force microscopy, spectroscopy for 350 nm absorbance, or Fourier transform infrared spectroscopy.

In one aspect, said at least one product quality comprises low molecular weight species and a performance target for low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said low molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE) or native polyacrylamide gel electrophoresis. In a more specific aspect, said SDS-PAGE is reducing SDS-PAGE or non-reducing SDS-PAGE.

In one aspect, said at least one product quality comprises non-glycosylated heavy chain and a performance target for non-glycosylated heavy chain is an upper limit of about 10%, about 9%, about 8%, about 7%, about 6%, about 5%, about 4%, about 3%, about 2%, or about 1%. In a specific aspect, said non-glycosylated heavy chain is measured by reduced capillary electrophoresis. In a more specific aspect, said reduced capillary electrophoresis is reduced microfluidic capillary electrophoresis.

In one aspect, said at least one product quality comprises charge variant profile and a performance target for charge variant profile is an upper limit for a charge variant of about 50%, about 45%, about 40%, about 35%, about 30%, about 25%, about 20%, or about 15%. In a specific aspect, said charge variant profile is measured by imaged capillary isoelectric focusing (iCIEF). In another specific aspect, said charge variant profile is measured by ion exchange chromatography or isoelectric focusing.

In one aspect, said at least one product quality comprises a charge variant and a performance target for said charge variant is an upper limit on variation from an average charge variant value of about 50%, about 45%, about 40%, about 35%, about 30%, about 25%, about 20%, about 15%, about 10%, about 5%, about 6 standard deviations, about 5 standard deviations, about 4 standard deviations, about 3 standard deviations, about 2 standard deviations, or about 1 standard deviation. In a specific aspect, said charge variant is measured by imaged capillary isoelectric focusing. In another specific aspect, said charge variant is measured by ion exchange chromatography or isoelectric focusing.

In one aspect, said at least one product quality comprises iCIEF Region 1 and a performance target for iCIEF Region 1 is an upper limit of about 60%, about 55%, about 50%, about 45%, about 40%, about 35%, or about 30%.

In one aspect, said at least one product quality comprises iCIEF Region 2 and a performance target for iCIEF Region 2 is a lower limit of about 30%, about 35%, about 40%, about 45%, about 50%, about 55%, or about 60%.

In one aspect, said at least one product quality comprises iCIEF Region 3 and a performance target for iCIEF Region 3 is an upper limit of about 40%, about 35%, about 30%, about 25%, about 20%, about 25%, about 20%, about 15%, or about 10%.

In one aspect, said at least one product quality comprises bispecific purity and a performance target for bispecific purity is a lower limit of about 90%, about 91%, about 92%, about 93%, about 94%, about 95%, about 95%, about 97%, about 98%, or about 99%. In a specific aspect, said bispecific purity is measured by protein A chromatography or mixed mode chromatography. In a more specific aspect, said chromatography comprises high performance liquid chromatography (HPLC) or ultra-performance liquid chromatography (UPLC). In another specific aspect, said bispecific purity is measured by liquid chromatography, capillary electrophoresis or gel electrophoresis. In an additional specific aspect, said bispecific impurity is measured by Protein A chromatography, Protein G chromatography, Protein L chromatography, hydrophobic interaction chromatography, mixed mode chromatography, and/or mass spectrometry.

In one aspect, said at least one product quality comprises binding impurity and a performance target for binding impurity is an upper limit of about 10%, about 9%, about 8%, about 7%, about 6%, about 5%, about 4%, about 3%, about 2%, or about 1%. In a specific aspect, said binding impurity is measured by protein A chromatography or mixed mode chromatography. In a more specific aspect, said chromatography comprises HPLC or UPLC. In another specific aspect, said binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said at least one product quality comprises non-binding impurity and a performance target for non-binding impurity is an upper limit of about 10%, about 9%, about 8%, about 7%, about 6%, about 5%, about 4%, about 3%, about 2%, or about 1%. In a specific aspect, said non-binding impurity is measured by protein A chromatography or mixed mode chromatography. In a more specific aspect, said chromatography comprises HPLC or UPLC. In another specific aspect, said non-binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said cells are selected from the group consisting of CHO cells, HEK293 cells, and BHK cells. In a specific aspect, said CHO cells are selected from the group consisting of CHO-K1, CHO DUX B-11, Veggie-CHO, GS-CHO, S-CHO, or CHO lec.

In one aspect, said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof. In another aspect, said recombinant protein is dupilumab.

In one aspect, said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.

In one aspect, said at least one DO excursion includes a relative DO of from 0% to 50%, from 0% to 25%, from 0% to 20%, from 0% to 10%, about 50%, about 25%, about 20%, about 15%, about 10%, about 9%, about 8%, about 7%, about 6%, about 5%, about 4%, about 3%, about 2%, about 1%, or about 0% of the DO setpoint. In a specific aspect, the DO setpoint is about 40%. In another specific aspect, the DO setpoint is about 25%.

In one aspect, said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.

In one aspect, said at least one DO excursion occurs during an early phase, a middle phase, and/or a late phase of said cell culture.

In one aspect, said at least one regression model comprises a random forest model, a batch evolution model and/or a batch level model.

In one aspect, the method further comprises producing said model, wherein producing said model comprises (a) culturing cells in a cell culture, wherein said cells produce a recombinant protein; (b) creating a DO excursion in said cell culture according to a first set of input parameters; (c) measuring a set of output parameters of said recombinant protein; (d) repeating steps (a)-(c) for additional sets of input parameters; and (e) subjecting the results of step (d) to statistical analysis correlating said DO excursions according to said input parameters to said output parameters to produce a model.

This disclosure also provides methods for modifying a cell culture process. In some exemplary aspects, the methods can comprise (a) culturing cells in a cell culture according to a first cell culture process, wherein said cells produce a recombinant protein and said cell culture undergoes at least one DO excursion; (b) predicting at least one recombinant protein parameter by using at least one regression model correlating said at least one DO excursion to said at least one recombinant protein parameter; and (c) culturing said cells according to a second cell culture process, wherein said second cell culture process is modified from said first cell culture process using said at least one predicted recombinant protein parameter.

In one aspect, modifying said first cell culture process comprises modifying a feed schedule, modifying a feed quantity, modifying an agitation rate, modifying a sparging rate, modifying a DO setpoint, and/or modifying a cell culture batch duration.

In one aspect, the method further comprises comparing each of said at least one recombinant protein parameters to a performance target for said recombinant protein parameter.

In one aspect, the at least one recombinant protein parameter is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one recombinant protein parameter is a product quality attribute. In still another aspect, said at least one recombinant protein parameter is a critical quality attribute.

In one aspect, said at least one recombinant protein parameter comprises protein titer and a performance target for said protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.

In one aspect, said at least one recombinant protein parameter comprises protein purity and a performance target for said protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%. In a specific aspect, said protein purity is measured by liquid chromatography, capillary electrophoresis, gel electrophoresis, or mass spectrometry. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is 1D or 2D sodium dodecyl sulfate-polyacrylamide gel electrophoresis.

In one aspect, said at least one recombinant protein parameter comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said high molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis. In a different specific aspect, said high molecular weight species are measured by size exclusion chromatography, native polyacrylamide gel electrophoresis, capillary electrophoresis, light scattering, dynamic light scattering, analytical ultracentrifugation, scanning electron microscopy, transmission electron microscopy, atomic force microscopy, spectroscopy for 350 nm absorbance, or Fourier transform infrared spectroscopy.

In one aspect, said at least one recombinant protein parameter comprises low molecular weight species and a performance target for said low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said low molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE) or native polyacrylamide gel electrophoresis. In a more specific aspect, said SDS-PAGE is reducing SDS-PAGE or non-reducing SDS-PAGE.

In one aspect, said at least one recombinant protein parameter comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%. In a specific aspect, said non-glycosylated heavy chain is measured by reduced capillary electrophoresis. In a more specific aspect, said reduced capillary electrophoresis is reduced microfluidic capillary electrophoresis.

In one aspect, said at least one recombinant protein parameter comprises charge variant profile and a performance target for said recombinant protein parameter is an upper limit for a charge variant of about 50%, about 45%, about 40%, about 35%, about 30%, about 25%, about 20%, or about 15%. In a specific aspect, said charge variant profile is measured by imaged capillary isoelectric focusing. In another specific aspect, said charge variant profile is measured by ion exchange chromatography or isoelectric focusing.

In one aspect, said at least one product quality comprises a charge variant and a performance target for said charge variant is an upper limit on variation from an average charge variant value of about 50%, about 45%, about 40%, about 35%, about 30%, about 25%, about 20%, about 15%, about 10%, about 5%, about 6 standard deviations, about 5 standard deviations, about 4 standard deviations, about 3 standard deviations, about 2 standard deviations, or about 1 standard deviation. In a specific aspect, said charge variant is measured by imaged capillary isoelectric focusing. In another specific aspect, said charge variant is measured by ion exchange chromatography or isoelectric focusing.

In one aspect, said at least one recombinant protein parameter comprises iCIEF Region 1 and a performance target for iCIEF Region 1 is an upper limit of about 60%, about 55%, about 50%, about 45%, about 40%, about 35%, or about 30%.

In one aspect, said at least one recombinant protein parameter comprises iCIEF Region 2 and a performance target for iCIEF Region 2 is a lower limit of about 30%, about 35%, about 40%, about 45%, about 50%, about 55%, or about 60%.

In one aspect, said at least one recombinant protein parameter comprises iCIEF Region 3 and a performance target for iCIEF Region 3 is an upper limit of about 40%, about 35%, about 30%, about 25%, about 20%, about 25%, about 20%, about 15%, or about 10%.

In one aspect, said at least one recombinant protein parameter comprises bispecific purity and a performance target for said bispecific purity is a lower limit of about 90%, about 91%, about 92%, about 93%, about 94%, about 95%, about 96%, about 97%, about 98%, or about 99%. In a specific aspect, said bispecific purity is measured by protein A chromatography or mixed mode chromatography. In a more specific aspect, said chromatography comprises HPLC or UPLC. In another specific aspect, said bispecific purity is measured by liquid chromatography, capillary electrophoresis or gel electrophoresis. In an additional specific aspect, said bispecific impurity is measured by Protein A chromatography, Protein G chromatography, Protein L chromatography, hydrophobic interaction chromatography, mixed mode chromatography, and/or mass spectrometry.

In one aspect, said at least one recombinant protein parameter comprises binding impurity and a performance target for said binding impurity is an upper limit of about 10%, about 9%, about 8%, about 7%, about 6%, about 5%, about 4%, about 3%, about 2%, or about 1%. In a specific aspect, said binding impurity is measured by protein A chromatography or mixed mode chromatography. In a more specific aspect, said chromatography comprises HPLC or UPLC. In another specific aspect, said binding impurity is measured by liquid chromatography, capillary electrophoresis or gel electrophoresis. In an additional specific aspect, said binding impurity is measured by Protein A chromatography, Protein G chromatography, Protein L chromatography, hydrophobic interaction chromatography, mixed mode chromatography, and/or mass spectrometry.

In one aspect, said at least one recombinant protein parameter comprises non-binding impurity and a performance target for said non-binding impurity is an upper limit of about 10%, about 9%, about 8%, about 7%, about 6%, about 5%, about 4%, about 3%, about 2%, or about 1%. In a specific aspect, said non-binding impurity is measured by protein A chromatography or mixed mode chromatography. In a more specific aspect, said chromatography comprises HPLC or UPLC. In another specific aspect, said non-binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said cells are selected from the group consisting of CHO cells, HEK293 cells, and BHK cells. In a specific aspect, said CHO cells are selected from the group consisting of CHO-K1, CHO DUX B-11, Veggie-CHO, GS-CHO, S-CHO, or CHO lec.

In one aspect, said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof. In another aspect, said recombinant protein is dupilumab.

In one aspect, said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.

In one aspect, said at least one DO excursion includes a relative DO of from 0% to 50%, from 0% to 25%, from 0% to 20%, from 0% to 10%, about 50%, about 25%, about 20%, about 15%, about 10%, about 9%, about 8%, about 7%, about 6%, about 5%, about 4%, about 3%, about 2%, about 1%, or about 0% of the DO setpoint. In a specific aspect, the DO setpoint is about 40%. In another specific aspect, the DO setpoint is about 25%.

In one aspect, said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.

In one aspect, said at least one DO excursion occurs during an early phase, a middle phase, and/or a late phase of said cell culture.

In one aspect, said at least one regression model comprises a random forest model, a batch evolution model and/or a batch level model.

This disclosure also provides systems for improved production of a recombinant protein. In some exemplary aspects, the systems can comprise (a) one or more vessels for culturing cells expressing a recombinant protein; (b) one or more probes for measuring one or more cell culture conditions in said one or more vessels; (c) one or more modules for controlling one or more settings in said one or more vessels; (d) a computing system coupled to said one or more probes and said one or more modules; (e) a prediction model stored in non-transitory machine-readable medium coupled to said computing system; and (f) a signal generator for controlling said one or more modules.

In one aspect, said one or more cell culture conditions are selected from a group consisting of dissolved oxygen levels, dissolved carbon dioxide levels, viable cell density, capacitance, pH, temperature, agitation rate, or sparging rate.

In one aspect, said one or more settings are selected from a group consisting of pH, temperature, agitation rates, sparging rates, nutrient feeds, fluid control valves, and/or batch duration.

In one aspect, said system further comprises one or more detectors for characterizing protein titer and/or product quality attributes. In a specific aspect, said characterizing comprises imaged capillary isoelectric focusing (iCIEF), capillary electrophoresis (CE), microfluidic capillary electrophoresis (MCE), size exclusion chromatography (SEC), size exclusion-ultra-performance liquid chromatography (SE-UPLC), high-performance liquid chromatography (HPLC), mass spectrometry (MS), tandem mass spectrometry (MS/MS), liquid chromatography-mass spectrometry (LC-MS), capillary electrophoresis-mass spectrometry (CE-MS), gel electrophoresis, nuclear magnetic resonance, and/or surface plasmon resonance.

In one aspect, said system further comprises a prediction model generator for generating a prediction model, comprising (a) one or more vessels for culturing cells expressing a recombinant protein; (b) one or more probes for measuring one or more cell culture conditions in said one or more vessels; (c) one or more detectors for characterizing protein titer and/or product quality attributes of said recombinant protein; (d) a computing system for receiving data input from said one or more probes and one or more detectors; and (e) instructions stored in non-transitory machine-readable medium that when processed by said computing system perform operations comprising: (i) correlating data inputs from said one or more probes with data inputs from said one or more detectors; (ii) generating a prediction model of protein titer and/or product quality attributes based on said correlation(s); and (iii) storing said prediction model in a database.

In a specific aspect, said operations further comprise: (iv) determining one or more settings for said one or more vessels using said prediction model; and (v) storing said determined setting(s), said data inputs from said one or more probes and said data inputs from said one or more detectors in said database.

In another specific aspect, said data input from said one or more probes of said prediction model generator includes a duration of at least one excursion of said one or more cell culture conditions, a magnitude of at least one excursion of said one or more cell culture conditions, and/or a timing of at least one excursion of said one or more cell culture conditions.

In a further specific aspect, said timing is selected from a group consisting of an early phase, a middle phase, and/or a late phase of said cell culture.

In another specific aspect, said one or more cell culture conditions of said prediction model generator are selected from a group consisting of dissolved oxygen, dissolved carbon dioxide, viable cell density, capacitance, pH, temperature, agitation rate, or sparging rate.

In an additional specific aspect, said one or more settings of said prediction model generator are selected from a group consisting of pH, temperature, agitation rates, sparging rates, nutrient feeds, fluid control valves, and/or batch duration.

In another specific aspect, said operations of said prediction model generator further comprise comparing said protein titer and/or said product quality attributes to a performance target, wherein said performance target is based on values set forth in good manufacturing practices (GMP) or regulatory standards.

In one aspect, the system further comprises a manual override disabling said signal generator. In another aspect, the system further comprises a report generator for generating a report from said prediction model.

In one aspect, said fluid control valves control transfer of said cells from one vessel to another vessel.

In one aspect, said prediction model comprises a random forest model, a batch evolution model and/or a batch level model.

In one aspect, said recombinant protein is dupilumab.

This disclosure provides additional methods for controlling the duration of a cell culture batch. In some exemplary aspects, the methods can comprise (a) culturing cells in a cell culture, wherein said cells produce a recombinant protein and said cell culture undergoes at least one dissolved oxygen (DO) excursion; (b) correlating the expression of at least one gene in said cells to at least one predicted product quality using at least one regression model; and (c) controlling the duration of said cell culture batch based on said at least one predicted product quality.

In one aspect, said controlling further comprises comparing each of said at least one predicted product qualities to a performance target for said product quality. In a specific aspect, said controlling further comprises terminating said cell culture batch when said at least one predicted product quality fails to meet said performance target. In another specific aspect, said at least one product quality comprises protein titer, said predicted protein titer is lower than a performance target for protein titer, and said controlling further comprises increasing a duration of said cell culture batch.

In one aspect, said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one product quality is a product quality attribute. In still another aspect, said at least one product quality is a critical quality attribute.

In one aspect, said at least one product quality comprises protein titer and a performance target for protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.

In one aspect, said at least one product quality comprises protein purity and a performance target for protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%. In a specific aspect, said protein purity is measured by liquid chromatography, capillary electrophoresis, gel electrophoresis, or mass spectrometry. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is 1D or 2D sodium dodecyl sulfate-polyacrylamide gel electrophoresis.

In one aspect, said at least one product quality comprises high molecular weight species and a performance target for high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said high molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE). In a different specific aspect, said high molecular weight species are measured by size exclusion chromatography, native polyacrylamide gel electrophoresis, capillary electrophoresis, light scattering, dynamic light scattering, analytical ultracentrifugation, scanning electron microscopy, transmission electron microscopy, atomic force microscopy, spectroscopy for 350 nm absorbance, or Fourier transform infrared spectroscopy.

In one aspect, said at least one product quality comprises low molecular weight species and a performance target for low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said low molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE) or native polyacrylamide gel electrophoresis. In a more specific aspect, said SDS-PAGE is reducing SDS-PAGE or non-reducing SDS-PAGE.

In one aspect, said at least one product quality comprises non-glycosylated heavy chain and a performance target for non-glycosylated heavy chain is an upper limit of about 10%, about 9%, about 8%, about 7%, about 6%, about 5%, about 4%, about 3%, about 2%, or about 1%. In a specific aspect, said non-glycosylated heavy chain is measured by reduced capillary electrophoresis. In a more specific aspect, said reduced capillary electrophoresis is reduced microfluidic capillary electrophoresis.

In one aspect, said at least one product quality comprises charge variant profile and a performance target for charge variant profile is an upper limit for a charge variant of about 50%, about 45%, about 40%, about 35%, about 30%, about 25%, about 20%, or about 15%. In a specific aspect, said charge variant profile is measured by imaged capillary isoelectric focusing (iCIEF). In another specific aspect, said charge variant profile is measured by ion exchange chromatography or isoelectric focusing.

In one aspect, said at least one product quality comprises a charge variant and a performance target for said charge variant is an upper limit on variation from an average charge variant value of about 50%, about 45%, about 40%, about 35%, about 30%, about 25%, about 20%, about 15%, about 10%, about 5%, about 6 standard deviations, about 5 standard deviations, about 4 standard deviations, about 3 standard deviations, about 2 standard deviations, or about 1 standard deviation. In a specific aspect, said charge variant is measured by imaged capillary isoelectric focusing. In another specific aspect, said charge variant is measured by ion exchange chromatography or isoelectric focusing.

In one aspect, said at least one product quality comprises iCIEF Region 1 and a performance target for iCIEF Region 1 is an upper limit of about 60%, about 55%, about 50%, about 45%, about 40%, about 35%, or about 30%.

In one aspect, said at least one product quality comprises iCIEF Region 2 and a performance target for iCIEF Region 2 is a lower limit of about 30%, about 35%, about 40%, about 45%, about 50%, about 55%, or about 60%.

In one aspect, said at least one product quality comprises iCIEF Region 3 and a performance target for iCIEF Region 3 is an upper limit of about 40%, about 35%, about 30%, about 25%, about 20%, about 25%, about 20%, about 15%, or about 10%.

In one aspect, said at least one product quality comprises bispecific purity and a performance target for bispecific purity is a lower limit of about 90%, about 91%, about 92%, about 93%, about 94%, about 95%, about 95%, about 97%, about 98%, or about 99%. In a specific aspect, said bispecific purity is measured by protein A chromatography or mixed mode chromatography. In a more specific aspect, said chromatography comprises high performance liquid chromatography (HPLC) or ultra-performance liquid chromatography (UPLC). In another specific aspect, said bispecific purity is measured by liquid chromatography, capillary electrophoresis or gel electrophoresis. In an additional specific aspect, said bispecific impurity is measured by Protein A chromatography, Protein G chromatography, Protein L chromatography, hydrophobic interaction chromatography, mixed mode chromatography, and/or mass spectrometry.

In one aspect, said at least one product quality comprises binding impurity and a performance target for binding impurity is an upper limit of about 10%, about 9%, about 8%, about 7%, about 6%, about 5%, about 4%, about 3%, about 2%, or about 1%. In a specific aspect, said binding impurity is measured by protein A chromatography or mixed mode chromatography. In a more specific aspect, said chromatography comprises HPLC or UPLC. In another specific aspect, said binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said at least one product quality comprises non-binding impurity and a performance target for non-binding impurity is an upper limit of about 10%, about 9%, about 8%, about 7%, about 6%, about 5%, about 4%, about 3%, about 2%, or about 1%. In a specific aspect, said non-binding impurity is measured by protein A chromatography or mixed mode chromatography. In a more specific aspect, said chromatography comprises HPLC or UPLC. In another specific aspect, said non-binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said cells are selected from the group consisting of CHO cells, HEK293 cells, and BHK cells. In a specific aspect, said CHO cells are selected from the group consisting of CHO-K1, CHO DUX B-11, Veggie-CHO, GS-CHO, S-CHO, or CHO lec.

In one aspect, said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof. In another aspect, said recombinant protein is dupilumab.

In one aspect, said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.

In one aspect, said at least one DO excursion includes a relative DO of from 0% to 50%, from 0% to 25%, from 0% to 20%, from 0% to 10%, about 50%, about 25%, about 20%, about 15%, about 10%, about 9%, about 8%, about 7%, about 6%, about 5%, about 4%, about 3%, about 2%, about 1%, or about 0% of the DO setpoint. In a specific aspect, the DO setpoint is about 40%. In another specific aspect, the DO setpoint is about 25%.

In one aspect, said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.

In one aspect, said at least one DO excursion occurs during an early phase, a middle phase, and/or a late phase of said cell culture.

In one aspect, said at least one regression model comprises a random forest model, a batch evolution model and/or a batch level model.

In one aspect, the expression of said at least one gene is measured using RNA sequencing (RNAseq), quantitative polymerase chain reaction (qPCR), or a microarray.

In one aspect, said at least one gene is a hypoxia-related gene. In another aspect, said at least one gene is a biomarker.

In one aspect, the method further comprises producing said model, wherein producing said model comprises (a) culturing cells in a cell culture, wherein said cells produce a recombinant protein; (b) creating a DO excursion in said cell culture according to a first set of input parameters; (c) measuring expression of at least one gene; (d) measuring a set of output parameters of said recombinant protein; (e) repeating steps (a)-(d) for additional sets of input parameters; and (f) subjecting the results of step (e) to statistical analysis correlating said expression of at least one gene to said output parameters to produce a model.

This disclosure also provides further methods for modifying a cell culture process. In some exemplary aspects, the methods can comprise (a) culturing cells in a cell culture according to a first cell culture process, wherein said cells produce a recombinant protein and said cell culture undergoes at least one DO excursion; (b) predicting at least one recombinant protein parameter by using at least one regression model correlating the expression of at least one gene in said cells to said at least one recombinant protein parameter; and (c) culturing said cells according to a second cell culture process, wherein said second cell culture process is modified from said first cell culture process using said at least one predicted recombinant protein parameter.

In one aspect, modifying said first cell culture process comprises modifying a feed schedule, modifying a feed quantity, modifying an agitation rate, modifying a sparging rate, modifying a DO setpoint, and/or modifying a cell culture batch duration.

In one aspect, the method further comprises comparing each of said at least one recombinant protein parameters to a performance target for said recombinant protein parameter.

In one aspect, the at least one recombinant protein parameter is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one recombinant protein parameter is a product quality attribute. In still another aspect, said at least one recombinant protein parameter is a critical quality attribute.

In one aspect, said at least one recombinant protein parameter comprises protein titer and a performance target for said protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.

In one aspect, said at least one recombinant protein parameter comprises protein purity and a performance target for said protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%. In a specific aspect, said protein purity is measured by liquid chromatography, capillary electrophoresis, gel electrophoresis, or mass spectrometry. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is 1D or 2D sodium dodecyl sulfate-polyacrylamide gel electrophoresis.

In one aspect, said at least one recombinant protein parameter comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said high molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis. In a different specific aspect, said high molecular weight species are measured by size exclusion chromatography, native polyacrylamide gel electrophoresis, capillary electrophoresis, light scattering, dynamic light scattering, analytical ultracentrifugation, scanning electron microscopy, transmission electron microscopy, atomic force microscopy, spectroscopy for 350 nm absorbance, or Fourier transform infrared spectroscopy.

In one aspect, said at least one recombinant protein parameter comprises low molecular weight species and a performance target for said low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said low molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE) or native polyacrylamide gel electrophoresis. In a more specific aspect, said SDS-PAGE is reducing SDS-PAGE or non-reducing SDS-PAGE.

In one aspect, said at least one recombinant protein parameter comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%. In a specific aspect, said non-glycosylated heavy chain is measured by reduced capillary electrophoresis. In a more specific aspect, said reduced capillary electrophoresis is reduced microfluidic capillary electrophoresis.

In one aspect, said at least one recombinant protein parameter comprises charge variant profile and a performance target for said recombinant protein parameter is an upper limit for a charge variant of about 50%, about 45%, about 40%, about 35%, about 30%, about 25%, about 20%, or about 15%. In a specific aspect, said charge variant profile is measured by imaged capillary isoelectric focusing. In another specific aspect, said charge variant profile is measured by ion exchange chromatography or isoelectric focusing.

In one aspect, said at least one product quality comprises a charge variant and a performance target for said charge variant is an upper limit on variation from an average charge variant value of about 50%, about 45%, about 40%, about 35%, about 30%, about 25%, about 20%, about 15%, about 10%, about 5%, about 6 standard deviations, about 5 standard deviations, about 4 standard deviations, about 3 standard deviations, about 2 standard deviations, or about 1 standard deviation. In a specific aspect, said charge variant is measured by imaged capillary isoelectric focusing. In another specific aspect, said charge variant is measured by ion exchange chromatography or isoelectric focusing.

In one aspect, said at least one recombinant protein parameter comprises iCIEF Region 1 and a performance target for iCIEF Region 1 is an upper limit of about 60%, about 55%, about 50%, about 45%, about 40%, about 35%, or about 30%.

In one aspect, said at least one recombinant protein parameter comprises iCIEF Region 2 and a performance target for iCIEF Region 2 is a lower limit of about 30%, about 35%, about 40%, about 45%, about 50%, about 55%, or about 60%.

In one aspect, said at least one recombinant protein parameter comprises iCIEF Region 3 and a performance target for iCIEF Region 3 is an upper limit of about 40%, about 35%, about 30%, about 25%, about 20%, about 25%, about 20%, about 15%, or about 10%.

In one aspect, said at least one recombinant protein parameter comprises bispecific purity and a performance target for said bispecific purity is a lower limit of about 90%, about 91%, about 92%, about 93%, about 94%, about 95%, about 96%, about 97%, about 98%, or about 99%. In a specific aspect, said bispecific purity is measured by protein A chromatography or mixed mode chromatography. In a more specific aspect, said chromatography comprises HPLC or UPLC. In another specific aspect, said bispecific purity is measured by liquid chromatography, capillary electrophoresis or gel electrophoresis. In an additional specific aspect, said bispecific impurity is measured by Protein A chromatography, Protein G chromatography, Protein L chromatography, hydrophobic interaction chromatography, mixed mode chromatography, and/or mass spectrometry.

In one aspect, said at least one recombinant protein parameter comprises binding impurity and a performance target for said binding impurity is an upper limit of about 10%, about 9%, about 8%, about 7%, about 6%, about 5%, about 4%, about 3%, about 2%, or about 1%. In a specific aspect, said binding impurity is measured by protein A chromatography or mixed mode chromatography. In a more specific aspect, said chromatography comprises HPLC or UPLC. In another specific aspect, said binding impurity is measured by liquid chromatography, capillary electrophoresis or gel electrophoresis. In an additional specific aspect, said binding impurity is measured by Protein A chromatography, Protein G chromatography, Protein L chromatography, hydrophobic interaction chromatography, mixed mode chromatography, and/or mass spectrometry.

In one aspect, said at least one recombinant protein parameter comprises non-binding impurity and a performance target for said non-binding impurity is an upper limit of about 10%, about 9%, about 8%, about 7%, about 6%, about 5%, about 4%, about 3%, about 2%, or about 1%. In a specific aspect, said non-binding impurity is measured by protein A chromatography or mixed mode chromatography. In a more specific aspect, said chromatography comprises HPLC or UPLC. In another specific aspect, said non-binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said cells are selected from the group consisting of CHO cells, HEK293 cells, and BHK cells. In a specific aspect, said CHO cells are selected from the group consisting of CHO-K1, CHO DUX B-11, Veggie-CHO, GS-CHO, S-CHO, or CHO lec.

In one aspect, said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof. In another aspect, said recombinant protein is dupilumab.

In one aspect, said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.

In one aspect, said at least one DO excursion includes a relative DO of from 0% to 50%, from 0% to 25%, from 0% to 20%, from 0% to 10%, about 50%, about 25%, about 20%, about 15%, about 10%, about 9%, about 8%, about 7%, about 6%, about 5%, about 4%, about 3%, about 2%, about 1%, or about 0% of the DO setpoint. In a specific aspect, the DO setpoint is about 40%. In another specific aspect, the DO setpoint is about 25%.

In one aspect, said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.

In one aspect, said at least one DO excursion occurs during an early phase, a middle phase, and/or a late phase of said cell culture.

In one aspect, said at least one regression model comprises a random forest model, a batch evolution model and/or a batch level model.

In one aspect, the expression of said at least one gene is measured using RNAseq, qPCR, or a microarray.

In one aspect, said at least one gene is a hypoxia-related gene. In another aspect, said at least one gene is a biomarker.

This disclosure also provides further systems for improved production of a recombinant protein. In some exemplary aspects, the systems can comprise (a) one or more vessels for culturing cells expressing a recombinant protein; (b) one or more ports for collecting one or more samples from said one or more vessels; (c) one or more analyzers for analyzing said one or more samples; (d) one or more modules for controlling one or more settings in said one or more vessels; (e) a computing system coupled to said one or more analyzers and said one or more modules; (f) a prediction model stored in non-transitory machine-readable medium coupled to said computing system; and (g) a signal generator for controlling said one or more modules.

In one aspect, said one or more analyzers are configured to analyze gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging 02%, temperature-corrected pH, temperature-corrected partial pressure of oxygen, temperature-corrected partial pressure of carbon dioxide, O₂ saturation, CO₂ saturation, and/or bicarbonate.

In one aspect, said one or more analyzers are coupled online to said one or more ports.

In one aspect, said one or more settings are selected from a group consisting of pH, temperature, agitation rates, sparging rates, nutrient feeds, fluid control valves, and/or batch duration.

In one aspect, said system further comprises one or more detectors for characterizing protein titer and/or product quality attributes. In a specific aspect, said characterizing comprises imaged capillary isoelectric focusing (iCIEF), capillary electrophoresis (CE), microfluidic capillary electrophoresis (MCE), size exclusion chromatography (SEC), size exclusion-ultra-performance liquid chromatography (SE-UPLC), high-performance liquid chromatography (HPLC), mass spectrometry (MS), tandem mass spectrometry (MS/MS), liquid chromatography-mass spectrometry (LC-MS), capillary electrophoresis-mass spectrometry (CE-MS), gel electrophoresis, nuclear magnetic resonance, and/or surface plasmon resonance.

In one aspect, said system further comprises a prediction model generator for generating a prediction model, comprising (a) one or more vessels for culturing cells expressing a recombinant protein; (b) one or more ports for collecting one or more samples from said one or more vessels; (c) one or more analyzers for analyzing said one or more samples; (d) one or more detectors for characterizing protein titer and/or product quality attributes of said recombinant protein; (e) a computing system for receiving data input from said one or more analyzers and one or more detectors; and (f) instructions stored in non-transitory machine-readable medium that when processed by said computing system perform operations comprising: (i) correlating data inputs from said one or more analyzers with data inputs from said one or more detectors; (ii) generating a prediction model of protein titer and/or product quality attributes based on said correlation(s); and (iii) storing said prediction model in a database.

In a specific aspect, said operations further comprise: (iv) determining one or more settings for said one or more vessels using said prediction model; and (v) storing said determined setting(s), said data inputs from said one or more analyzers and said data inputs from said one or more detectors in said database.

In another specific aspect, said data input from said one or more analyzers of said prediction model generator includes quantification of gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O₂%, temperature-corrected pH, temperature-corrected partial pressure of oxygen, temperature-corrected partial pressure of carbon dioxide, O₂ saturation, CO₂ saturation, and/or bicarbonate.

In an additional specific aspect, said one or more analyzers are coupled online to said one or more ports.

In a further specific aspect, said one or more settings of said prediction model generator are selected from a group consisting of pH, temperature, agitation rates, sparging rates, nutrient feeds, fluid control valves, and/or batch duration.

In another specific aspect, said operations of said prediction model generator further comprise comparing said protein titer and/or said product quality attributes to a performance target, wherein said performance target is based on values set forth in good manufacturing practices (GMP) or regulatory standards.

In one aspect, the system further comprises a manual override disabling said signal generator. In another aspect, the system further comprises a report generator for generating a report from said prediction model.

In one aspect, said fluid control valves control transfer of said cells from one vessel to another vessel.

In one aspect, said prediction model comprises a random forest model, a batch evolution model and/or a batch level model.

In one aspect, said recombinant protein is dupilumab.

This disclosure provides additional methods for controlling the duration of a cell culture batch. In some exemplary aspects, the methods can comprise (a) culturing cells in a cell culture, wherein said cells produce a recombinant protein and said cell culture undergoes at least one dissolved oxygen (DO) excursion; (b) correlating at least one cell culture health parameter to at least one predicted product quality using at least one regression model; and (c) controlling the duration of said cell culture batch based on said at least one predicted product quality.

In one aspect, said controlling further comprises comparing each of said at least one predicted product qualities to a performance target for said product quality. In a specific aspect, said controlling further comprises terminating said cell culture batch when said at least one predicted product quality fails to meet said performance target. In another specific aspect, said at least one product quality comprises protein titer, said predicted protein titer is lower than a performance target for protein titer, and said controlling further comprises increasing a duration of said cell culture batch.

In one aspect, said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one product quality is a product quality attribute. In still another aspect, said at least one product quality is a critical quality attribute.

In one aspect, said at least one product quality comprises protein titer and a performance target for protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.

In one aspect, said at least one product quality comprises protein purity and a performance target for protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%. In a specific aspect, said protein purity is measured by liquid chromatography, capillary electrophoresis, gel electrophoresis, or mass spectrometry. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is 1D or 2D sodium dodecyl sulfate-polyacrylamide gel electrophoresis.

In one aspect, said at least one product quality comprises high molecular weight species and a performance target for high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said high molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE). In a different specific aspect, said high molecular weight species are measured by size exclusion chromatography, native polyacrylamide gel electrophoresis, capillary electrophoresis, light scattering, dynamic light scattering, analytical ultracentrifugation, scanning electron microscopy, transmission electron microscopy, atomic force microscopy, spectroscopy for 350 nm absorbance, or Fourier transform infrared spectroscopy.

In one aspect, said at least one product quality comprises low molecular weight species and a performance target for low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said low molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE) or native polyacrylamide gel electrophoresis. In a more specific aspect, said SDS-PAGE is reducing SDS-PAGE or non-reducing SDS-PAGE.

In one aspect, said at least one product quality comprises non-glycosylated heavy chain and a performance target for non-glycosylated heavy chain is an upper limit of about 10%, about 9%, about 8%, about 7%, about 6%, about 5%, about 4%, about 3%, about 2%, or about 1%. In a specific aspect, said non-glycosylated heavy chain is measured by reduced capillary electrophoresis. In a more specific aspect, said reduced capillary electrophoresis is reduced microfluidic capillary electrophoresis.

In one aspect, said at least one product quality comprises charge variant profile and a performance target for charge variant profile is an upper limit for a charge variant of about 50%, about 45%, about 40%, about 35%, about 30%, about 25%, about 20%, or about 15%. In a specific aspect, said charge variant profile is measured by imaged capillary isoelectric focusing (iCIEF). In another specific aspect, said charge variant profile is measured by ion exchange chromatography or isoelectric focusing.

In one aspect, said at least one product quality comprises a charge variant and a performance target for said charge variant is an upper limit on variation from an average charge variant value of about 50%, about 45%, about 40%, about 35%, about 30%, about 25%, about 20%, about 15%, about 10%, about 5%, about 6 standard deviations, about 5 standard deviations, about 4 standard deviations, about 3 standard deviations, about 2 standard deviations, or about 1 standard deviation. In a specific aspect, said charge variant is measured by imaged capillary isoelectric focusing. In another specific aspect, said charge variant is measured by ion exchange chromatography or isoelectric focusing.

In one aspect, said at least one product quality comprises iCIEF Region 1 and a performance target for iCIEF Region 1 is an upper limit of about 60%, about 55%, about 50%, about 45%, about 40%, about 35%, or about 30%.

In one aspect, said at least one product quality comprises iCIEF Region 2 and a performance target for iCIEF Region 2 is a lower limit of about 30%, about 35%, about 40%, about 45%, about 50%, about 55%, or about 60%.

In one aspect, said at least one product quality comprises iCIEF Region 3 and a performance target for iCIEF Region 3 is an upper limit of about 40%, about 35%, about 30%, about 25%, about 20%, about 25%, about 20%, about 15%, or about 10%.

In one aspect, said at least one product quality comprises bispecific purity and a performance target for bispecific purity is a lower limit of about 90%, about 91%, about 92%, about 93%, about 94%, about 95%, about 95%, about 97%, about 98%, or about 99%. In a specific aspect, said bispecific purity is measured by protein A chromatography or mixed mode chromatography. In a more specific aspect, said chromatography comprises high performance liquid chromatography (HPLC) or ultra-performance liquid chromatography (UPLC). In another specific aspect, said bispecific purity is measured by liquid chromatography, capillary electrophoresis or gel electrophoresis. In an additional specific aspect, said bispecific impurity is measured by Protein A chromatography, Protein G chromatography, Protein L chromatography, hydrophobic interaction chromatography, mixed mode chromatography, and/or mass spectrometry.

In one aspect, said at least one product quality comprises binding impurity and a performance target for binding impurity is an upper limit of about 10%, about 9%, about 8%, about 7%, about 6%, about 5%, about 4%, about 3%, about 2%, or about 1%. In a specific aspect, said binding impurity is measured by protein A chromatography or mixed mode chromatography. In a more specific aspect, said chromatography comprises HPLC or UPLC. In another specific aspect, said binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said at least one product quality comprises non-binding impurity and a performance target for non-binding impurity is an upper limit of about 10%, about 9%, about 8%, about 7%, about 6%, about 5%, about 4%, about 3%, about 2%, or about 1%. In a specific aspect, said non-binding impurity is measured by protein A chromatography or mixed mode chromatography. In a more specific aspect, said chromatography comprises HPLC or UPLC. In another specific aspect, said non-binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said cells are selected from the group consisting of CHO cells, HEK293 cells, and BHK cells. In a specific aspect, said CHO cells are selected from the group consisting of CHO-K1, CHO DUX B-11, Veggie-CHO, GS-CHO, S-CHO, or CHO lec.

In one aspect, said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof. In another aspect, said recombinant protein is dupilumab.

In one aspect, said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.

In one aspect, said at least one DO excursion includes a relative DO of from 0% to 50%, from 0% to 25%, from 0% to 20%, from 0% to 10%, about 50%, about 25%, about 20%, about 15%, about 10%, about 9%, about 8%, about 7%, about 6%, about 5%, about 4%, about 3%, about 2%, about 1%, or about 0% of the DO setpoint. In a specific aspect, the DO setpoint is about 40%. In another specific aspect, the DO setpoint is about 25%.

In one aspect, said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.

In one aspect, said at least one DO excursion occurs during an early phase, a middle phase, and/or a late phase of said cell culture.

In one aspect, said at least one regression model comprises a random forest model, a batch evolution model and/or a batch level model.

In one aspect, said at least one cell culture health parameter comprises one or more of pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O₂%, temperature-corrected pH, temperature-corrected partial pressure of oxygen, temperature-corrected partial pressure of carbon dioxide, O₂ saturation, CO₂ saturation, and/or bicarbonate.

In one aspect, the method further comprises producing said model, wherein producing said model comprises (a) culturing cells in a cell culture, wherein said cells produce a recombinant protein; (b) creating a DO excursion in said cell culture according to a first set of input parameters; (c) measuring at least one cell culture health parameter in said cell culture; (d) measuring a set of output parameters of said recombinant protein; (e) repeating steps (a)-(d) for additional sets of input parameters; and (f) subjecting the results of step (e) to statistical analysis correlating said at least one cell culture health parameter to said output parameters to produce a model.

This disclosure also provides methods for modifying a cell culture process. In some exemplary aspects, the methods can comprise (a) culturing cells in a cell culture according to a first cell culture process, wherein said cells produce a recombinant protein and said cell culture undergoes at least one DO excursion; (b) predicting at least one recombinant protein parameter by using at least one regression model correlating at least one cell culture health parameter to said at least one recombinant protein parameter; and (c) culturing said cells according to a second cell culture process, wherein said second cell culture process is modified from said first cell culture process using said at least one predicted recombinant protein parameter.

In one aspect, modifying said first cell culture process comprises modifying a feed schedule, modifying a feed quantity, modifying an agitation rate, modifying a sparging rate, modifying a DO setpoint, and/or modifying a cell culture batch duration.

In one aspect, the method further comprises comparing each of said at least one recombinant protein parameters to a performance target for said recombinant protein parameter.

In one aspect, the at least one recombinant protein parameter is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one recombinant protein parameter is a product quality attribute. In still another aspect, said at least one recombinant protein parameter is a critical quality attribute.

In one aspect, said at least one recombinant protein parameter comprises protein titer and a performance target for said protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.

In one aspect, said at least one recombinant protein parameter comprises protein purity and a performance target for said protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%. In a specific aspect, said protein purity is measured by liquid chromatography, capillary electrophoresis, gel electrophoresis, or mass spectrometry. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is 1D or 2D sodium dodecyl sulfate-polyacrylamide gel electrophoresis.

In one aspect, said at least one recombinant protein parameter comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said high molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis. In a different specific aspect, said high molecular weight species are measured by size exclusion chromatography, native polyacrylamide gel electrophoresis, capillary electrophoresis, light scattering, dynamic light scattering, analytical ultracentrifugation, scanning electron microscopy, transmission electron microscopy, atomic force microscopy, spectroscopy for 350 nm absorbance, or Fourier transform infrared spectroscopy.

In one aspect, said at least one recombinant protein parameter comprises low molecular weight species and a performance target for said low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%. In a specific aspect, said low molecular weight species are measured by liquid chromatography, capillary electrophoresis or gel electrophoresis. In a more specific aspect, said liquid chromatography is size exclusion chromatography. In a further specific aspect, said size exclusion chromatography is size exclusion-ultra-performance liquid chromatography. In another specific aspect, said capillary electrophoresis is microfluidic capillary electrophoresis. In an additional specific aspect, said capillary electrophoresis is non-reduced capillary electrophoresis or reduced capillary electrophoresis. In another specific aspect, said gel electrophoresis is sodium dodecyl sulfate-polyacrylamide gel electrophoresis (SDS-PAGE) or native polyacrylamide gel electrophoresis. In a more specific aspect, said SDS-PAGE is reducing SDS-PAGE or non-reducing SDS-PAGE.

In one aspect, said at least one recombinant protein parameter comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%. In a specific aspect, said non-glycosylated heavy chain is measured by reduced capillary electrophoresis. In a more specific aspect, said reduced capillary electrophoresis is reduced microfluidic capillary electrophoresis.

In one aspect, said at least one recombinant protein parameter comprises charge variant profile and a performance target for said recombinant protein parameter is an upper limit for a charge variant of about 50%, about 45%, about 40%, about 35%, about 30%, about 25%, about 20%, or about 15%. In a specific aspect, said charge variant profile is measured by imaged capillary isoelectric focusing. In another specific aspect, said charge variant profile is measured by ion exchange chromatography or isoelectric focusing.

In one aspect, said at least one product quality comprises a charge variant and a performance target for said charge variant is an upper limit on variation from an average charge variant value of about 50%, about 45%, about 40%, about 35%, about 30%, about 25%, about 20%, about 15%, about 10%, about 5%, about 6 standard deviations, about 5 standard deviations, about 4 standard deviations, about 3 standard deviations, about 2 standard deviations, or about 1 standard deviation. In a specific aspect, said charge variant is measured by imaged capillary isoelectric focusing. In another specific aspect, said charge variant is measured by ion exchange chromatography or isoelectric focusing.

In one aspect, said at least one recombinant protein parameter comprises iCIEF Region 1 and a performance target for iCIEF Region 1 is an upper limit of about 60%, about 55%, about 50%, about 45%, about 40%, about 35%, or about 30%.

In one aspect, said at least one recombinant protein parameter comprises iCIEF Region 2 and a performance target for iCIEF Region 2 is a lower limit of about 30%, about 35%, about 40%, about 45%, about 50%, about 55%, or about 60%.

In one aspect, said at least one recombinant protein parameter comprises iCIEF Region 3 and a performance target for iCIEF Region 3 is an upper limit of about 40%, about 35%, about 30%, about 25%, about 20%, about 25%, about 20%, about 15%, or about 10%.

In one aspect, said at least one recombinant protein parameter comprises bispecific purity and a performance target for said bispecific purity is a lower limit of about 90%, about 91%, about 92%, about 93%, about 94%, about 95%, about 96%, about 97%, about 98%, or about 99%. In a specific aspect, said bispecific purity is measured by protein A chromatography or mixed mode chromatography. In a more specific aspect, said chromatography comprises HPLC or UPLC. In another specific aspect, said bispecific purity is measured by liquid chromatography, capillary electrophoresis or gel electrophoresis. In an additional specific aspect, said bispecific impurity is measured by Protein A chromatography, Protein G chromatography, Protein L chromatography, hydrophobic interaction chromatography, mixed mode chromatography, and/or mass spectrometry.

In one aspect, said at least one recombinant protein parameter comprises binding impurity and a performance target for said binding impurity is an upper limit of about 10%, about 9%, about 8%, about 7%, about 6%, about 5%, about 4%, about 3%, about 2%, or about 1%. In a specific aspect, said binding impurity is measured by protein A chromatography or mixed mode chromatography. In a more specific aspect, said chromatography comprises HPLC or UPLC. In another specific aspect, said binding impurity is measured by liquid chromatography, capillary electrophoresis or gel electrophoresis. In an additional specific aspect, said binding impurity is measured by Protein A chromatography, Protein G chromatography, Protein L chromatography, hydrophobic interaction chromatography, mixed mode chromatography, and/or mass spectrometry.

In one aspect, said at least one recombinant protein parameter comprises non-binding impurity and a performance target for said non-binding impurity is an upper limit of about 10%, about 9%, about 8%, about 7%, about 6%, about 5%, about 4%, about 3%, about 2%, or about 1%. In a specific aspect, said non-binding impurity is measured by protein A chromatography or mixed mode chromatography. In a more specific aspect, said chromatography comprises HPLC or UPLC. In another specific aspect, said non-binding impurity is measured by liquid chromatography, capillary electrophoresis or gel electrophoresis. In an additional specific aspect, said non-binding impurity is measured by Protein A chromatography, Protein G chromatography, Protein L chromatography, hydrophobic interaction chromatography, mixed mode chromatography, and/or mass spectrometry.

In one aspect, said cells are selected from the group consisting of CHO cells, HEK293 cells, and BHK cells. In a specific aspect, said CHO cells are selected from the group consisting of CHO-K1, CHO DUX B-11, Veggie-CHO, GS-CHO, S-CHO, or CHO lec.

In one aspect, said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof. In another aspect, said recombinant protein is dupilumab.

In one aspect, said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.

In one aspect, said at least one DO excursion includes a relative DO of from 0% to 50%, from 0% to 25%, from 0% to 20%, from 0% to 10%, about 50%, about 25%, about 20%, about 15%, about 10%, about 9%, about 8%, about 7%, about 6%, about 5%, about 4%, about 3%, about 2%, about 1%, or about 0% of the DO setpoint. In a specific aspect, the DO setpoint is about 40%. In another specific aspect, the DO setpoint is about 25%.

In one aspect, said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.

In one aspect, said at least one DO excursion occurs during an early phase, a middle phase, and/or a late phase of said cell culture.

In one aspect, said at least one regression model comprises a random forest model, a batch evolution model and/or a batch level model.

In one aspect, said at least one cell culture health parameter comprises one or more of pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O₂%, temperature-corrected pH, temperature-corrected partial pressure of oxygen, temperature-corrected partial pressure of carbon dioxide, O₂ saturation, CO₂ saturation, and/or bicarbonate.

This disclosure also provides methods for predicting at least one property of a cell culture batch. In some exemplary aspects, the methods can comprise (a) measuring at least one condition of a cell culture batch, wherein said cells produce a recombinant protein and said cell culture batch undergoes at least one DO excursion; and (b) predicting at least one property of said cell culture batch by using at least one regression model correlating said at least one condition to said at least one property.

In one aspect, said at least one condition is one or more of DO excursion magnitude, DO excursion duration, DO excursion timing, gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O₂%, temperature-corrected pH, temperature-corrected partial pressure of oxygen, temperature-corrected partial pressure of carbon dioxide, O₂ saturation, CO₂ saturation, and bicarbonate.

In one aspect, said at least one property is one or more of viable cell density, viability, protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and terminal galactosylation. In another aspect, said at least one property is a product quality attribute. In still another aspect, said at least one property is a critical quality attribute.

In one aspect, the methods can further comprise (c) modifying a cell culture process for said cell culture batch using said prediction of said at least one property. In a specific aspect, modifying said cell culture process comprises modifying a feed schedule, modifying a feed quantity, modifying an agitation rate, modifying a sparging rate, modifying a DO setpoint, and/or modifying a cell culture batch duration.

In one aspect, said recombinant protein is dupilumab.

This disclosure additionally provides methods for improving dupilumab production. In some exemplary aspects, the methods can comprise (a) culturing cells in a cell culture according to a first cell culture process, wherein said cells produce dupilumab and said cell culture undergoes at least one DO excursion; (b) predicting at least one dupilumab product quality by using at least one regression model correlating at least one cell culture condition to said at least one product quality; and (c) culturing said cells according to a second cell culture process, wherein said second cell culture process is modified from said first cell culture process using said at least one predicted product quality to improve dupilumab production.

In one aspect, said at least one cell culture condition is one or more of DO excursion magnitude, DO excursion duration, DO excursion timing, gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O₂%, temperature-corrected pH, temperature-corrected partial pressure of oxygen, temperature-corrected partial pressure of carbon dioxide, O₂ saturation, CO₂ saturation, and bicarbonate.

In one aspect, modifying said first cell culture process comprises modifying a feed schedule, modifying a feed quantity, modifying an agitation rate, modifying a sparging rate, modifying a DO setpoint, and/or modifying a cell culture batch duration.

In one aspect, the methods further comprise comparing each of said at least one product quality to a performance target for said product quality.

In one aspect, said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one product quality is a product quality attribute. In still another aspect, said at least one product quality is a critical quality attribute.

In one aspect, said cells are selected from the group consisting of CHO cells, HEK293 cells, and BHK cells. In another aspect, said cells are selected from the group consisting of CHO-K1, CHO DUX B-11, Veggie-CHO, GS-CHO, S-CHO, or CHO lec.

In one aspect, said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.

In one aspect, said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.

In one aspect, said at least one DO excursion occurs during an early phase, a middle phase, and/or a late phase of said cell culture.

In one aspect, said at least one regression model comprises a random forest model, a batch evolution model and/or a batch level model.

This disclosure further provides methods for identifying a biomarker of a recombinant protein parameter. In some exemplary aspects, the methods can comprise (a) growing cells in at least two cell cultures, wherein said cells express a recombinant protein, at least one of said at least two cell cultures undergoes a dissolved oxygen excursion, and at least one of said at least two cell cultures does not undergo a dissolved oxygen excursion; (b) measuring expression of one or more genes in said cells; (c) measuring a parameter of said recombinant protein; and (d) correlating said measured expression to said measured parameter using at least one regression model, wherein the expression of at least one of said one or more genes correlates to said measured parameter, thereby identifying a biomarker.

In one aspect, the at least one recombinant protein parameter is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation. In another aspect, said at least one recombinant protein parameter is a product quality attribute. In still another aspect, said at least one recombinant protein parameter is a critical quality attribute.

In one aspect, said cells are selected from the group consisting of CHO cells, HEK293 cells, and BHK cells. In a specific aspect, said CHO cells are selected from the group consisting of CHO-K1, CHO DUX B-11, Veggie-CHO, GS-CHO, S-CHO, or CHO lec.

In one aspect, said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof. In another aspect, said recombinant protein is dupilumab.

In one aspect, said at least one regression model comprises a random forest model. In a specific aspect, the method further comprises measuring feature importance to identify said biomarker.

These, and other, aspects of the invention will be better appreciated and understood when considered in conjunction with the following description and accompanying drawings. The following description, while indicating various aspects and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1A shows viable cell density (VCD) of a cell culture batch subjected to a late phase 24-hour excursion at 0% DO compared to an average of control batches (center point, CP), according to an exemplary aspect.

FIG. 1B shows viability of a cell culture batch subjected to a late phase 24-hour excursion at 0% DO compared to an average of control batches, according to an exemplary aspect.

FIG. 1C shows titer of a product produced in a cell culture batch subjected to a late phase 24-hour excursion at 0% DO compared to an average of control batches, according to an exemplary aspect.

FIG. 2A shows VCD of a cell culture batch subjected to an early phase 24-hour excursion at 8% DO compared to an average of control batches, according to an exemplary aspect.

FIG. 2B shows viability of a cell culture batch subjected to an early phase 24-hour excursion at 8% DO compared to an average of control batches, according to an exemplary aspect.

FIG. 2C shows titer of a product produced in a cell culture batch subjected to an early phase 24-hour excursion at 8% DO compared to an average of control batches, according to an exemplary aspect.

FIG. 3A shows VCD of a cell culture batch subjected to a late phase 96-hour excursion at 8% DO compared to an average of control batches, according to an exemplary aspect.

FIG. 3B shows viability of a cell culture batch subjected to a late phase 96-hour excursion at 8% DO compared to an average of control batches, according to an exemplary aspect.

FIG. 3C shows titer of a product produced in a cell culture batch subjected to a late phase 96-hour excursion at 8% DO compared to an average of control batches, according to an exemplary aspect.

FIG. 4A shows VCD of a cell culture batch subjected to 42-hour early, middle, and late phase excursions at 8% DO compared to an average of control batches, according to an exemplary aspect.

FIG. 4B shows viability of a cell culture batch subjected to 42-hour early, middle, and late phase excursions at 8% DO compared to an average of control batches, according to an exemplary aspect.

FIG. 4C shows titer of a product produced in a cell culture batch subjected to 42-hour early, middle, and late phase excursions at 8% DO compared to an average of control batches, according to an exemplary aspect.

FIG. 5A shows VCD of a cell culture batch subjected to 96-hour early, middle, and late phase excursions at 8% DO compared to an average of control batches, according to an exemplary aspect.

FIG. 5B shows viability of a cell culture batch subjected to 96-hour early, middle, and late phase excursions at 8% DO compared to an average of control batches, according to an exemplary aspect.

FIG. 5C shows titer of a product produced in a cell culture batch subjected to 96-hour early, middle, and late phase excursions at 8% DO compared to an average of control batches, according to an exemplary aspect.

FIG. 6A shows VCD of a cell culture batch subjected to 60-hour early, middle, and late phase excursions at 4% DO compared to an average of control batches, according to an exemplary aspect.

FIG. 6B shows viability of a cell culture batch subjected to 60-hour early, middle, and late phase excursions at 4% DO compared to an average of control batches, according to an exemplary aspect.

FIG. 6C shows titer of a product produced in a cell culture batch subjected to 60-hour early, middle, and late phase excursions at 4% DO compared to an average of control batches, according to an exemplary aspect.

FIG. 7A shows VCD of a cell culture batch subjected to 75-hour early and middle phase excursions at 5.7% DO compared to an average of control batches, according to an exemplary aspect.

FIG. 7B shows viability of a cell culture batch subjected to 75-hour early and middle phase excursions at 5.7% DO compared to an average of control batches, according to an exemplary aspect.

FIG. 7C shows titer of a product produced in a cell culture batch subjected to 75-hour early and middle phase excursions at 5.7% DO compared to an average of control batches, according to an exemplary aspect.

FIG. 8A shows VCD of a cell culture batch subjected to 76-hour middle and late phase excursions at 5.6% DO compared to an average of control batches, according to an exemplary aspect.

FIG. 8B shows viability of a cell culture batch subjected to 76-hour middle and late phase excursions at 5.6% DO compared to an average of control batches, according to an exemplary aspect.

FIG. 8C shows titer of a product produced in a cell culture batch subjected to 76-hour middle and late phase excursions at 5.6% DO compared to an average of control batches, according to an exemplary aspect.

FIG. 9A shows VCD of a cell culture batch subjected to 24-hour early, middle, and late phase excursions at 0% DO compared to an average of control batches, according to an exemplary aspect.

FIG. 9B shows viability of a cell culture batch subjected to 24-hour early, middle, and late phase excursions at 0% DO compared to an average of control batches, according to an exemplary aspect.

FIG. 9C shows titer of a product produced in a cell culture batch subjected to 24-hour early, middle, and late phase excursions at 0% DO compared to an average of control batches, according to an exemplary aspect.

FIG. 10A shows VCD of a cell culture batch subjected to 96-hour early, middle, and late phase excursions at 0% DO compared to an average of control batches, according to an exemplary aspect.

FIG. 10B shows viability of a cell culture batch subjected to 96-hour early, middle, and late phase excursions at 0% DO compared to an average of control batches, according to an exemplary aspect.

FIG. 10C shows titer of a product produced in a cell culture batch subjected to 96-hour early, middle, and late phase excursions at 0% DO compared to an average of control batches, according to an exemplary aspect.

FIG. 11A shows VCD of a cell culture batch subjected to 96-hour early and middle phase excursions at 0% DO compared to an average of control batches, according to an exemplary aspect.

FIG. 11B shows viability of a cell culture batch subjected to 96-hour early and middle phase excursions at 0% DO compared to an average of control batches, according to an exemplary aspect.

FIG. 11C shows titer of a product produced in a cell culture batch subjected to 96-hour early and middle phase excursions at 0% DO compared to an average of control batches, according to an exemplary aspect.

FIG. 12A shows the purity by size exclusion-ultra-performance liquid chromatography (SE-UPLC) of products produced in cell culture batches subjected to various dissolved oxygen (DO) excursions compared to a performance target, according to an exemplary aspect.

FIG. 12B shows high molecular weight (HMW) species by SE-UPLC of products produced in cell culture batches subjected to various DO excursions compared to a performance target, according to an exemplary aspect.

FIG. 12C shows imaged capillary isoelectric focusing (iCIEF) Region 1 of products produced in cell culture batches subjected to various DO excursions compared to a performance target, according to an exemplary aspect.

FIG. 12D shows iCIEF Region 2 of products produced in cell culture batches subjected to various DO excursions compared to a performance target, according to an exemplary aspect.

FIG. 12E shows iCIEF Region 3 of products produced in cell culture batches subjected to various DO excursions compared to a performance target, according to an exemplary aspect.

FIG. 12F shows purity by non-reduced (NR) microfluidic capillary electrophoresis (MCE) of products produced in cell culture batches subjected to various DO excursions compared to a performance target, according to an exemplary aspect.

FIG. 12G shows low molecular weight (LMW) species by NR MCE of products produced in cell culture batches subjected to various DO excursions compared to a performance target, according to an exemplary aspect.

FIG. 12H shows purity by reduced (R) MCE of products produced in cell culture batches subjected to various DO excursions compared to a performance target, according to an exemplary aspect.

FIG. 12I shows LMW by R MCE of products produced in cell culture batches subjected to various DO excursions compared to a performance target, according to an exemplary aspect.

FIG. 12J shows non-glycosylated heavy chain (NGHC) by R MCE of products produced in cell culture batches subjected to various DO excursions compared to a performance target, according to an exemplary aspect.

FIG. 13A shows a predicted final viability compared to actual final viability, according to an exemplary aspect.

FIG. 13B shows a three-dimensional (3D) response surface for a regression model for predicting final viability based on excursion length and DO % for a middle phase excursion, according to an exemplary aspect.

FIG. 14A shows a predicted final titer compared to actual final titer, according to an exemplary aspect.

FIG. 14B shows a normalized 3D response surface for a regression model for predicting final protein titer based on excursion length and DO % for an early phase excursion, according to an exemplary aspect.

FIG. 14C shows a normalized 3D response surface for a regression model for predicting final protein titer based on excursion length and DO % for a middle phase excursion, according to an exemplary aspect.

FIG. 14D shows a normalized 3D response surface for a regression model for predicting final protein titer based on excursion length and DO % for a late phase excursion, according to an exemplary aspect.

FIG. 15A shows purity by NR MCE and R MCE of products produced in cell culture batches subjected to various DO excursions, according to an exemplary aspect.

FIG. 15B shows a predicted protein purity by NR MCE compared to actual protein purity by NR MCE, according to an exemplary aspect.

FIG. 15C shows a 3D response surface for a regression model for predicting protein purity by NR MCE based on excursion length and DO % for a middle phase excursion, according to an exemplary aspect.

FIG. 16A shows LMW by NR MCE, LMW by R MCE, and NGHC by R MCE of products produced in cell culture batches subjected to various DO excursions, according to an exemplary aspect.

FIG. 16B shows predicted LMW by NR MCE compared to actual LMW by NR MCE, according to an exemplary aspect.

FIG. 16C shows a 3D response surface for a regression model for predicting LMW by NR MCE based on excursion length and DO % for a middle phase excursion, according to an exemplary aspect.

FIG. 17A shows iCIEF Regions 1, 2, and 3 of products produced in cell culture batches subjected to various DO excursions, according to an exemplary aspect.

FIG. 17B shows predicted iCIEF Region 1(%) compared to actual iCIEF Region 1(%), according to an exemplary aspect.

FIG. 17C shows a 3D response surface for a regression model for predicting iCIEF Region 1(%) based on excursion length and DO % for a middle phase excursion, according to an exemplary aspect.

FIG. 17D shows predicted iCIEF Region 2(%) compared to actual iCIEF Region 2(%), according to an exemplary aspect.

FIG. 17E shows a 3D response surface for a regression model for predicting iCIEF Region 2(%) based on excursion length and DO % for a middle phase excursion, according to an exemplary aspect.

FIG. 17F shows predicted iCIEF Region 3(%) compared to actual iCIEF Region 3(%), according to an exemplary aspect.

FIG. 17G shows a 3D response surface for a regression model for predicting iCIEF Region 3(%) based on excursion length and DO % for a middle phase excursion, according to an exemplary aspect.

FIG. 18A shows fucosylated glycans of products produced in cell culture batches subjected to various DO excursions, according to an exemplary aspect.

FIG. 18B shows predicted fucosylated glycans compared to actual fucosylated glycans, according to an exemplary aspect.

FIG. 18C shows a 3D response surface for a regression model for predicting fucosylated glycans based on excursion length and DO % for a middle phase excursion, according to an exemplary aspect.

FIG. 19A shows a prediction profile for determining the lowest acceptable DO % for a 96-hour early phase excursion, wherein product acceptability is limited by a performance target of NGHC as measured by R MCE, according to an exemplary aspect.

FIG. 19B shows a prediction profile for determining the lowest acceptable DO % for a 24-hour early phase excursion, wherein product acceptability is limited by a performance target of LMW as measured by NR MCE, according to an exemplary aspect.

FIG. 19C shows a prediction profile for determining the longest acceptable duration for a middle phase excursion at 8% DO, wherein product acceptability is limited by a performance target of NGHC as measured by R MCE, according to an exemplary aspect.

FIG. 19D shows a prediction profile for determining the lowest acceptable DO % for a 24-hour middle phase excursion, wherein product acceptability is limited by a performance target of LMW as measured by NR MCE, according to an exemplary aspect.

FIG. 19E shows a prediction profile for determining the longest acceptable duration for a late phase excursion at 8% DO, wherein product acceptability is limited by a performance target of NGHC as measured by R MCE, according to an exemplary aspect.

FIG. 19F shows a prediction profile for determining the longest acceptable duration for a late phase excursion at 8% DO, wherein product acceptability is limited by a performance target of LMW as measured by NR MCE, according to an exemplary aspect.

FIG. 19G shows a prediction profile for determining the lowest acceptable DO % for a 24-hour late phase excursion, wherein product acceptability is limited by a performance target of LMW as measured by NR MCE, according to an exemplary aspect.

FIG. 20A shows peak VCD for dupilumab production batches subjected to various DO excursions compared to an upper performance target (UPT), according to an exemplary aspect.

FIG. 20B shows prediction profiles for peak VCD of dupilumab production batches as a function of exemplary significant model terms, according to an exemplary aspect.

FIG. 21 shows final cell culture viability for dupilumab production batches subjected to various DO excursions compared to a lower performance target (LPT), according to an exemplary aspect.

FIG. 22A shows final titer for dupilumab production batches subjected to various DO excursions compared to performance targets, according to an exemplary aspect.

FIG. 22B shows prediction profiles for final titer of dupilumab production batches as a function of exemplary significant model terms, according to an exemplary aspect.

FIG. 23 shows Chinese Hamster Ovary (CHO) host cell protein (HCP) content for dupilumab production batches subjected to various DO excursions compared to a performance target, according to an exemplary aspect.

FIG. 24 shows purity by R MCE for dupilumab production batches subjected to various DO excursions compared to a performance target, according to an exemplary aspect.

FIG. 25 shows LMW by R MCE for dupilumab production batches subjected to various DO excursions compared to a performance target, according to an exemplary aspect.

FIG. 26A shows NGHC by R MCE for dupilumab production batches subjected to various DO excursions compared to a performance target, according to an exemplary aspect.

FIG. 26B shows prediction profiles for NGHC by R MCE of dupilumab production batches as a function of exemplary significant model terms, according to an exemplary aspect.

FIG. 27A shows purity by NR MCE for dupilumab production batches subjected to various DO excursions compared to a performance target, according to an exemplary aspect.

FIG. 27B shows prediction profiles for purity by NR MCE of dupilumab production batches as a function of exemplary significant model terms, according to an exemplary aspect.

FIG. 28A shows LMW by NR MCE for dupilumab production batches subjected to various DO excursions compared to a performance target, according to an exemplary aspect.

FIG. 28B shows prediction profiles for LMW by NR MCE of dupilumab production batches as a function of exemplary significant model terms, according to an exemplary aspect.

FIG. 29A shows purity by SE-UPLC for dupilumab production batches subjected to various DO excursions compared to a performance target, according to an exemplary aspect.

FIG. 29B shows prediction profiles for purity by SE-UPLC of dupilumab production batches as a function of exemplary significant model terms, according to an exemplary aspect.

FIG. 30A shows HMW by SE-UPLC for dupilumab production batches subjected to various DO excursions compared to a performance target, according to an exemplary aspect.

FIG. 30B shows prediction profiles for HMW by SE-UPLC of dupilumab production batches as a function of exemplary significant model terms, according to an exemplary aspect.

FIG. 31A shows iCIEF Region 1 for dupilumab production batches subjected to various DO excursions compared to performance targets, according to an exemplary aspect.

FIG. 31B shows prediction profiles for iCIEF Region 1 of dupilumab production batches as a function of exemplary significant model terms, according to an exemplary aspect.

FIG. 32A shows iCIEF Region 2 for dupilumab production batches subjected to various DO excursions compared to performance targets, according to an exemplary aspect.

FIG. 32B shows prediction profiles for iCIEF Region 2 of dupilumab production batches as a function of exemplary significant model terms, according to an exemplary aspect.

FIG. 33 shows iCIEF Region 3 for dupilumab production batches subjected to various DO excursions compared to a performance target, according to an exemplary aspect.

FIG. 34 shows fucosylated glycans for dupilumab production batches subjected to various DO excursions compared to a performance target, according to an exemplary aspect.

FIG. 35 shows G2+G2F for dupilumab production batches subjected to various DO excursions, according to an exemplary aspect.

FIG. 36 shows mannose-5 for dupilumab production batches subjected to various DO excursions, according to an exemplary aspect.

FIG. 37A shows G0+G0F for dupilumab production batches subjected to various DO excursions, according to an exemplary aspect.

FIG. 37B shows prediction profiles for G0+G0F of dupilumab production batches as a function of exemplary significant model terms, according to an exemplary aspect.

FIG. 38A shows G1+G1F for dupilumab production batches subjected to various DO excursions, according to an exemplary aspect.

FIG. 38B shows prediction profiles for G1+G1F of dupilumab production batches as a function of exemplary significant model terms, according to an exemplary aspect.

FIG. 39A shows an overview of predicted means for LMW by NR MCE for Drug Product A within the design space for early phase excursions, according to an exemplary aspect.

FIG. 39B shows an overview of predicted means for LMW by NR MCE for Drug Product A within the design space for middle phase excursions, according to an exemplary aspect.

FIG. 39C shows an overview of predicted means for LMW by NR MCE for Drug Product A within the design space for late phase excursions, according to an exemplary aspect.

FIG. 40A shows an overview of predicted means for NGHC by R MCE for Drug Product A within the design space for early phase excursions, according to an exemplary aspect.

FIG. 40B shows an overview of predicted means for NGHC by R MCE for Drug Product A within the design space for middle phase excursions, according to an exemplary aspect.

FIG. 40C shows an overview of predicted means for NGHC by R MCE for Drug Product A within the design space for late phase excursions, according to an exemplary aspect.

FIG. 41 illustrates a workflow for developing models for predicting cell culture process performance based on gene expression analysis, according to an exemplary aspect.

FIG. 42 illustrates how varying bioreactor conditions can result in varying gene expression levels, according to an exemplary aspect.

FIG. 43A shows a heat map visualization of gene expression over time across cell batches under control conditions, according to an exemplary aspect.

FIG. 43B shows changes in differentially expressed genes between each day of a cell culture process under control conditions, according to an exemplary aspect.

FIG. 43C shows changes in differentially expressed genes across each phase of a cell culture process under control conditions, according to an exemplary aspect.

FIG. 44A shows changes in expression of an exemplary gene, aurora kinase B (AURKB), in terms of transcripts per million (TPM) over time under control conditions, according to an exemplary aspect.

FIG. 44B shows changes in relative expression of genes related to hypoxia in terms of normalized enrichment score over time under control conditions, according to an exemplary aspect.

FIG. 45 shows a heat map visualization of differentially expressed genes under varying dissolved oxygen excursion conditions over time, according to an exemplary aspect.

FIG. 46A shows a heat map visualization of differentially expressed hypoxia-related genes under varying dissolved oxygen excursion conditions over time, according to an exemplary aspect.

FIG. 46B shows a comparison of differentially expressed genes from varying cellular pathways in terms of normalized enrichment score (NES) under control conditions (center point), during an excursion, or after an excursion, according to an exemplary aspect.

FIG. 47A shows glucan branching enzyme 1 (GBE1) expression levels across cell culture batches grouped by recombinant protein titer, according to an exemplary aspect.

FIG. 47B shows GBE1 expression levels across cell culture batches grouped by fucosylated glycan levels, according to an exemplary aspect.

FIG. 48 shows a principal component analysis (PCA) plot using transcriptomic markers at day 4 to group cell culture batches that passed versus failed product quality specifications, according to an exemplary aspect.

FIG. 49 shows a volcano plot identifying relevant genes that were differentially expressed between cell culture batches that passed versus failed product quality specifications, according to an exemplary aspect.

FIG. 50A shows a comparison of glutathione S-transferase pi 3 (GSTP3) expression across cell culture conditions and outcomes, according to an exemplary aspect.

FIG. 50B shows a comparison of superoxide dismutase 1 (SOD1) expression across cell culture conditions and outcomes, according to an exemplary aspect.

FIG. 50C shows a comparison of solute carrier family 7 member 11 (SLC7A11) expression across cell culture conditions and outcomes, according to an exemplary aspect.

FIG. 50D shows a comparison of phosphatidylinositol transfer protein membrane-associated 1 (PITPNM1) expression across cell culture conditions and outcomes, according to an exemplary aspect.

FIG. 51A shows a comparison of viable cell density for a control batch versus a dead batch that was subjected to a critical dissolved oxygen excursion, according to an exemplary aspect.

FIG. 51B shows a comparison of viability for a control batch versus a dead batch that was subjected to a critical dissolved oxygen excursion, according to an exemplary aspect.

FIG. 51C shows a comparison of recombinant protein titer for a control batch versus a dead batch that was subjected to a critical dissolved oxygen excursion, according to an exemplary aspect.

FIG. 52 shows a principal component analysis (PCA) plot using transcriptomic markers at day 4 to group cell culture batches that passed versus failed product quality specifications, according to an exemplary aspect.

FIG. 53 shows a volcano plot identifying relevant genes that were differentially expressed between cell culture batches that passed product quality specifications versus died out prior to harvest, according to an exemplary aspect.

FIG. 54A shows a comparison of solute carrier family 2 member 1 (SLC2A1) expression across cell culture conditions and outcomes, according to an exemplary aspect.

FIG. 54B shows a comparison of dual specificity phosphatase 8 (DUSP8) expression across cell culture conditions and outcomes, according to an exemplary aspect.

FIG. 54C shows a comparison of glucan branching enzyme 1 (GBE1) expression across cell culture conditions and outcomes, according to an exemplary aspect.

FIG. 54D shows a comparison of ankyrin repeat domain 37 (ANKRD37) expression across cell culture conditions and outcomes, according to an exemplary aspect.

FIG. 55 shows a comparison of predicted viable cell density (VCD) using random forest modeling from transcriptomic data compared to actual VCD, according to an exemplary aspect.

FIG. 56 shows an analysis of feature importance for predicting VCD using random forest modeling from transcriptomic data, according to an exemplary aspect.

FIG. 57 shows a comparison of predicted recombinant protein titer using random forest modeling from transcriptomic data compared to actual titer, according to an exemplary aspect.

FIG. 58A shows a comparison of predicted protein purity by NR MCE using random forest modeling from transcriptomic data compared to actual purity, according to an exemplary aspect.

FIG. 58B shows an analysis of feature importance for predicting protein purity by NR MCE using random forest modeling from transcriptomic data, according to an exemplary aspect.

FIG. 59A shows a comparison of predicted product quality specification passes and fails using random forest modeling from transcriptomic data compared to actual passes and fails, according to an exemplary aspect.

FIG. 59B shows an analysis of feature importance for predicting product quality using random forest modeling from transcriptomic data, according to an exemplary aspect.

FIG. 59C shows a multidimensional scaling plot grouping cell culture batches that passed versus failed product quality specifications, according to an exemplary aspect.

FIG. 60A shows a comparison of predicted versus observed LMW by NR MCE using a multivariate model based on cell culture health data, according to an exemplary aspect.

FIG. 60B shows a comparison of predicted versus observed LMW by NR MCE using a multivariate model based on transcriptomic data, according to an exemplary aspect.

FIG. 60C shows a comparison of predicted versus observed iCIEF Region 3 using a multivariate model based on cell culture health data, according to an exemplary aspect.

FIG. 60D shows a comparison of predicted versus observed iCIEF Region 3 using a multivariate model based on transcriptomic data, according to an exemplary aspect.

### DETAILED DESCRIPTION

Manufacturing processes for therapeutic proteins require strict control over manufacturing conditions in order to ensure the safety and efficacy of the resulting therapeutic product. Understanding the acceptable and desired operating ranges of critical process parameters (CPPs) is primarily achieved using proven acceptable range (PAR) studies. PAR studies help characterize the process by testing the limits of CPPs. Relevant input parameters are pushed outside of the normal operating range to evaluate process performance and determine the edge of failure. Acceptable ranges are then calculated based on where critical quality attributes (CQAs) will meet required specifications.

However, conventional PAR studies do not address transient excursions. Production bioreactors used in manufacturing suites often experience transient excursions during normal operations, including dissolved oxygen (DO) excursions. These excursions require investigations within the quality system, which lead to inefficiencies in time and resources. Because the specific relationship between the characteristics of DO excursions and the resulting impact on cell culture properties and protein quality are not yet understood, proteins must be harvested, purified and evaluated before it can be determined whether an observed DO excursion led to a product that fails to meet performance targets or specification limits.

In order to address this issue, a novel strategy was developed using design of experiments (DOE) screening of dissolved oxygen excursions to characterize the production bioreactor process of recombinant proteins, in order to predict the effect of DO excursions on product quality before having to harvest the protein. DO excursions were studied during the production bioreactor unit operation of two exemplary recombinant proteins, mAb1 and dupilumab. Data was collected and analyzed to characterize the cells and resulting product quality from bioreactors that experience DO excursions during production, as defined by the relationship between selected input and output parameters.

Several input parameters around DO excursions were examined. Commonly experienced DO excursions were evaluated, as well as those that may have the most impact to a batch (long excursions) versus the most impact to the compliance system (short or oscillatory excursions). The selected input parameters include duration of DO excursion, timing of DO excursion, and magnitude of DO excursion.

Using the duration of DO excursions as an input parameter provides insight into how long a DO excursion can last before it impacts the cell culture to a point at which product quality is affected. Fluctuations could cause changes in cellular metabolism, cell growth, and/or cell death.

Studying the timing of DO excursions elucidates which phases of production (for example, logarithmic growth, stationary phase, or death) are most sensitive to low DO excursions. The phase of the bioreactor when the DO excursion occurs could impact cellular metabolism, cell growth, and/or cell death, and affect titer and product quality. Dividing the design space into three discrete phases (early, middle, and late, corresponding to logarithmic growth, stationary, and death/decline, respectively) that reflect stereotypical discrete phases of cell metabolism and life cycle allows for distinguishing the varied effects of low oxygen on cells at biologically distinct times, while limiting the number of time points to an experimentally achievable quantity.

Investigating the magnitude of DO excursions helps demonstrate how low the DO must drop before a statistically significant change in product quality is observed. Variations could cause differential changes in cellular metabolism, cell growth, and/or cell death, and may differentially affect product quality.

The same parameters described above can be framed in different terms, for example as four input parameters including DO setpoint, duration of DO excursion in early phase, duration of DO excursion in middle phase, and duration of DO excursion in late phase.

Additional input parameters can be selected based on the process of interest; for example, batch length, feed schedule, agitation rate, or post-excursion DO concentration.

Differences in process performance caused by variations in excursion input parameters provide an indication of process robustness to DO excursions. Supplementary data from cell pellets (containing RNA and/or protein) and spent media can be further processed and analyzed for gene/protein expression and metabolite levels, respectively, to improve process understanding. Additional performance data summarizing culture conditions during each batch, such as profiles of nutrient, metabolite, gas, and/or electrolyte concentrations, can be trended to provide additional process understanding and to better characterize the full range of operability within the evaluated space.

Output parameters were selected after considering the relative importance of each in determining process consistency and/or product safety and efficacy. Output parameters included in-process DO traces; peak viable cell density (VCD); final cell viability; final titer; antibody purity, non-glycosylated heavy chain (NGHC) and low molecular weight (LMW) species as measured by reduced microfluidic capillary electrophoresis (MCE); antibody purity, LMW species, and high molecular weight (HMW) species as measured by non-reduced MCE; main peak purity and HMW species as measured by size exclusion-ultra-performance liquid chromatography (SE-UPLC); charge variant profile; glycosylation profile; and Chinese Hamster Ovary (CHO) host cell protein (HCP) content. Additional output parameters could be selected based on the particular product; for example, sequence variants, particular post-translational modifications, bispecific antibody purity, binding impurity, and non-binding impurity.

It should be understood that any suitable means may be used to measure output parameters, including, for example, liquid chromatography (for example, affinity chromatography, protein A chromatography, protein G chromatography, protein L chromatography, reversed phase liquid chromatography (RPLC), ion exchange chromatography (IEX) (for example, anion exchange chromatography (AEX) or cation exchange chromatography (CEX)), size-exclusion chromatography (SEC), hydrophobic interaction chromatography (HIC), hydrophilic interaction chromatography (HILIC), and mixed-mode chromatography (MMC)), electrophoresis (for example, gel electrophoresis, 2D gel electrophoresis, sodium dodecyl sulfate-polyacrylamide gel electrophoresis (SDS-PAGE), 2D SDS-PAGE, native polyacrylamide gel electrophoresis, capillary electrophoresis (CE), free flow electrophoresis (FFE), isoelectric focusing gel electrophoresis (IEF), capillary isoelectric focusing electrophoresis (CIEF), imaged capillary isoelectric focusing electrophoresis (iCIEF), and microfluidic capillary electrophoresis (MCE)), mass spectrometry (for example, tandem mass spectrometry (MS/MS), electrospray ionization-mass spectrometry (ESI-MS), nano-electrospray ionization mass spectrometry (nESI-MS), microflow nano-electrospray ionization mass spectrometer (MnESI-MS), inductively coupled plasma mass spectrometry (ICP-MS), high-resolution mass spectrometry (HRMS)), spectrophotometry, spectroscopy (for example, Fourier transform infrared (FTIR) spectroscopy), fluorescence detection, light scattering, dynamic light scattering (DLS), analytical ultracentrifugation (AUC), electron microscopy (for example, scanning electron microscopy (SEM) and transmission electron microscopy (TEM)), atomic force microscopy, and combinations thereof (for example, LC-UV-MS and CE-MS)).

As used herein, the term “product quality” may correspond to a defined product quality attribute (PQA), critical quality attribute (CQA), critical product quality attribute (cPQA), or may correspond to a property that is not conventionally considered a PQA or CQA, such as a process performance parameter directly related to a protein product. For example, the disclosed inventions include systems and methods for predicting protein titer and using those predictions to improve a manufacturing process, which are encompassed under the general term of product quality.

Multiple design of experiments screenings were performed to assess the impact of transient DO excursions on process performance and product quality. For a cell culture process producing the recombinant monoclonal antibody mAb1, results showed that titer is affected by all selected input parameters, with late-stage DO excursions resulting in minimal impact. Product quality is directly influenced by the magnitude and duration of DO excursions, with only extreme excursions causing specification failures and moderate conditions yielding acceptable antibodies. For a cell culture process producing another monoclonal antibody, dupilumab, additional excursion parameters were tested, and significant models were also produced for predicting dupilumab product quality as a result of DO excursions.

Further, gene expression changes related to normal recombinant protein production and to dissolved oxygen excursions were characterized, and biomarkers were identified for relating differential gene expression to particular product quality outcomes. Models were produced with the ability to predict final product quality on the basis of expression of genetic biomarkers as early as day 4 of a production batch.

Additionally, multivariate models were produced correlating gene expression changes or various continuous measures of cell culture health (for example, temperature, pH, dissolved gasses, and metabolite concentrations) to cell culture outcomes in batches subjected to varying DO excursions. Both types of model were able to reliably predict recombinant protein production process outcomes, demonstrating the ability to use easily measured cell culture properties as a powerful indicator of future production batch success.

The disclosed systems and methods allow for correlating measured DO excursions, measured biomarker expression, and/or measured cell culture health parameters to predicted product quality outcomes. Accordingly, the disclosed systems and methods further allow for the proactive determination of, for example, when a manufacturing process should be modified to accommodate a batch that is still within specification limits after an excursion, or when a batch should be terminated in order to prevent the loss of time and expense on a production batch that is predicted to fail specifications.

The systems and methods disclosed herein may further be applied to additional excursions encountered in cell culture and protein manufacturing, for example, pH excursions, CO2 excursions, temperature excursions, sparging excursions, agitation excursions, or feed-related excursions. In some aspects, determining a cell culture process comprises measuring more than one cell culture condition, using the more than one measurements to predict an effect of the measured cell culture conditions on cell culture health, protein titer or product quality, and using the prediction to modify the cell culture process. In particular, bioreactor conditions that are typically subjected to continuous measurements—such as DO, CO2, pH, temperature, capacitance, agitation rate, and sparging rate—can be used as a convenient and continuous basis for predicting a final protein titer and product quality, and these predictions may inform a decision of modifying a cell culture process in order to ensure meeting performance targets even after a cell culture batch experiences process excursions, or terminating a cell culture batch. In other aspects, determining a cell culture process comprises measuring the expression of one or more biomarkers, using the one or more measurements to predict an effect of the measured expression on cell culture health, protein titer or product quality, and using the prediction to modify the cell culture process.

Models may additionally be produced to predict cell culture health, product titer and/or product quality wherein different phases of cell culture are affected by excursions of varying durations and varying magnitudes (for example, DO %), for example by superimposing individual models produced for each excursion. A model may be particularly useful for predicting outcomes for a specific molecule, or a single model may be applicable to multiple molecules. In some aspects, a model derived from a first cell culture process producing a first molecule may be used to produce reliable predictions for a second cell culture process producing a second molecule. In particular aspects, the model may further be used to produce reliable predictions for a third process for a third molecule, and more.

These, and other aspects of the invention, are set forth in further detail below.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, bispecific antibodies, and antigen-binding proteins.

Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

The term “antibody,” as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In some aspects, the protein of interest is a human antibody. The term “human antibody,” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies of the disclosure may, in some aspects, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain aspects, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR), as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

An “isolated antibody,” as used herein, is intended to refer to an antibody that is substantially free of other antibodies having different antigenic specificities (e.g., an isolated antibody that specifically binds hIL-4Rα (human interleukin-4 receptor alpha) is substantially free of antibodies that specifically bind antigens other than hIL-4Rα).

The phrase “Fc-containing protein” includes antibodies, bispecific antibodies, antibody derivatives containing an Fc, antibody fragments containing an Fc, Fc-fusion proteins, receptor Fc-fusion proteins (including trap proteins), immunoadhesins, and other binding proteins that comprise at least a functional portion of an immunoglobulin CH2 and CH3 region. A “functional portion” refers to a CH2 and CH3 region that can bind an Fc receptor (for example, an FcyR; or an FcRn (neonatal Fc receptor)), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion, and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains. IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology among the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

The inventions are amenable to use with a wide variety of Fc-containing proteins and other proteins. The inventions can be employed in the production of biological and pharmaceutical products. For example, for antibodies, the inventions are amenable for research and production use for diagnostics and therapeutics based upon all major antibody classes, namely IgG, IgA, IgM, IgD, and IgE. IgG is a preferred class, and includes subclasses IgG1 (including IgG1λ and IgG1κ), IgG2, IgG3, and IgG4. In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. Derivatives, components, domains, chains, and fragments of the above are also included.

An abbreviated list of exemplary antibodies to be produced according to the inventions include Alirocumab, Atoltivimab, Maftivimab, Odesivimab, Odesivivmab-ebgn, Casirivimab, Imdevimab, Cemiplimab, Cemplimab-rwlc, Davutamig, Dupilumab, Evinacumab, Evinacumab-dgnb, Fasinumab, Nesvacumab, Trevogrumab, Rinucumab and Sarilumab. A longer list of exemplary antibodies to be produced according to the inventions include, but are not limited to, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Fianlimab, Garetosmab, Golimumab, Guselkumab, Ibritumomab tiuxetan, Idarucizumab, Infliximab, Infliximab-abda, Infliximab-dyyb, Ipilimumab, Itepekimab, Ixekizumab, Linvoseltamab, Linvoseltamab-gcpt, Mepolizumab, Mibavademab, Necitumumab, Nesvacumab, Nezastomig, Nivolumab, Obiltoxaximab, Obinutuzumab, Odronextamab, Ocrelizumab, Oatumumab, Olaratumab, Omalizumab, Panitumumab, Pembrolizumab (lambrolizumab), Pertuzumab, Pozelimab, Pozelimab-bbfg, Ramucirumab, Ranibizumab, Ravulizumab-cwvz, Raxibacumab, Reslizumab, Rituximab, Secukinumab, Siltuximab, Tocilizumab, Tocilizumab, Trastuzumab, Trevogrumab, Ubamatamab, Ustekinumab, Vedolizumab, Vonsetamig, anrukinzumab, atczolizumab, atlizumab, alacizumab pegol, etrolizumab, efalizumab, obexelimab, gantenerumab, inclacumab, brolucizumab, batoclimab, cedelizumab, crenezumab, Enoblituzumab, glycooptimized trastuzumab-GEX, Trastuzumab emtansine HER2, gemtuzumab, gemtuzumab ozogamicin, nimotuzumab, palivizumab, baciliximab, daclizumab, natalizumab, otelixizumab, teplizumab, epratuzumab, briakinumab, abagovomab, adecatumumab, afutuzumab, altumomab pentetate, amatuximab, anatumomab mafenatox, anetumab ravtansine, apolizumab, apomab, arcitumomab, ascrinvacumab, bavituximab, bectumomab, besilesomab, bivatuzumab mertansine, Brontictuzumab, cantuzumab mertansine, cantuzumab ravtansine, carlumab, catumaxomab, cBR-doxorubicin immunoconjugate, citatuzumab bogatox, cixutumumab, clenoliximab, clivatuzumab tetraxetan, codrituzumab, coltuximab ravtansine, conatumumab, dacetuzumab, dalotuzumab, dalotuzumab, daratumumab, demcizumab, denintuzumab mafodotin, depatuxizumab, derlotuximab, detumomab, drozitumab, duligotumab, duligotuzumab, dusigitumab, ecromeximab, edrecolomab, elgemtumab, elsilimomab, emactuzumab, emibetuzumab, emibetuzumab, enavatuzumab, enfortumab vedotin, enoticumab, ensituximab, epitumomab cituxetan, ertumaxomab, etaracizumab, faralimomab, farletuzumab, FBTA (CD20 x CD3), ficlatuzumab, figitumumab, flanvotumab, fresolimumab, futuximab, galiximab, gantiumab, gatipotuzumab, girentuximab, glembatumumab vedotin, icrucumab, igovomab, IMAB362 (CLDN18.2), imgatuzumab, indatuximab ravtansine, indusatumab vedotin, inebilizumab, inotuzumab ozogamicin, intetumumab, iratumumab, isatuximab, ipritumomab labetuzumab, lampalizumab, lenzilumab, lexatumumab, lifastuzumab vedotin, lilotomab satetraxetan, lebrikizumab, ligelizumab, lintuzumab, lirilumab, loncastuximab tesirine, lonvastuximab, lorvotuzumab mertansine, lucatumumab, lumiliximab, lumretuzumab, mapatumumab, margetuximab, matuzumab, milatuzumab, minretumomab, mirvetuximab soravtansine, mitumomab, mogamulizumab, moxetumomab pasudotox, muromonab-CD3, nacolomab tafenatox, naptumomab estafenatox, narnatumab, nofetumomab merpentan, binutuzumab, ocaratuzumab, onartuzumab, ontuxizumab, oportuzumab monatox, oregovomab, otlertuzumab, pankomab, parsatuzumab, pasotuxizumab, patritumab, pemtumomab, pidilizumab, pinatuzumab vedotin, pintumomab, polatuzumab vedotin, quilizumab, racotumomab, radretumab, rilotumumab, robatumumab, romosozumab, rontalizumab, sacituzumab govitecan, samalizumab, satralizumab, satumomab pendetide, seribantumab, seribantumab, SGN-CDA (CD19), SGN-CDA (CD33), sibrotuzumab, simtuzumab, siplizumab, sofituzumab vedotin, solitomab, sonepcizumab, tabalumab, tacatuzumab tetraxetan, tafasitamab, taplitumomab paptox, tarextumab, tenatumomab, teneliximab, teprotumumab, tetulomab, TGN (CD28), tigatuzumab, Timigutuzumab, tomuzotuximab, tositumomab, tovetumab (CD140a), tovetumab (PDGFRα), TRBS (GD2), tralokinumab, Tremelimumab, Ticilimumab, tucotuzumab celmoleukin, ublituximab (MS4A1), ublituximab (CD20), ulocuplumab, urelumab, vandortuzumab vedotin, vantictumab, vanucizumab, varlilumab, veltuzumab, vesencumab, visilizumab, volociximab, vorsetuzumab, votumumab, zalutumumab, zanolimumab, zatuximab, and ziralimumab.

In some aspects, the antibody is selected from the group consisting of an anti-Programmed Cell Death 1 antibody (e.g. an anti-PD1 antibody as described in U.S. Pat. No. 9,987,500), an anti-Programmed Cell Death Ligand-1 antibody (e.g. an anti-PD-L1 antibody as described in in U.S. Pat. No. 9,938,345), an anti-DII4 antibody, an anti-Angiopoietin-2 antibody (e.g. an anti-ANG2 antibody as described in U.S. Pat. No. 9,402,898), an anti-Angiopoietin-Like 3 antibody (e.g. an anti-AngPt13 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. No. 9,795,121), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. No. 9,475,875), an anti-Proprotein Convertase Subtilisin Kexin-9 antibody (e.g. an anti-PCSK9 antibody as described in U.S. Pat. No. 8,062,640 or U.S. Pat. No. 9,540,449), an anti-Growth And Differentiation Factor-8 antibody (e.g. an anti-GDF8 antibody, also known as anti-myostatin antibody, as described in U.S. Pat. No. 8,871,209 or U.S. Pat. No. 9,260,515), an anti-Glucagon Receptor (e.g. anti-GCGR antibody as described in U.S. Pat. No. 9,587,029 or U.S. Pat. No. 9,657,099), an anti-VEGF antibody, an anti-IL1R antibody, an interleukin 4 receptor antibody (e.g., an anti-IL4R antibody as described in U.S. Pat. Nos. 12,162,943, 8,735,095 or 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. No. 9,453,072 or U.S. Pat. No. 9,637,535), an anti-Cluster of differentiation 3 antibody (e.g. an anti-CD3 antibody as described in U.S. Pat. Nos. 9,657,102 and 10,550,193, and in U.S. Pat. App. Pub. No. US20210253701 A1), an anti-Cluster of differentiation 20 antibody (e.g. an anti-CD20 antibody as described in U.S. Pat. Nos. 9,657,102, 10,550,193, and 7,879,984), an anti-CD19 antibody, an anti-CD28 antibody, an anti-Cluster of Differentiation-48 antibody (e.g. anti-CD48 antibody as described in U.S. Pat. No. 9,228,014), an anti-Fel d1 antibody (e.g. as described in U.S. Pat. No. 9,079,948), an anti-influenza virus antibody, an anti-Respiratory syncytial virus antibody (e.g. anti-RSV antibody as described in U.S. Pat. App. Pub. No. US2014/0271653A1), an anti-Middle East Respiratory Syndrome virus antibody (e.g. an anti-MERS antibody as described in U.S. Pat. No. 9,718,872), an anti-Ebola virus antibody (e.g. as described in U.S. Pat. No. 9,771,414), an anti-Zika virus antibody, an anti-Severe Acute Respiratory Syndrome (SARS) antibody (e.g., an anti-SARS-COV antibody), an anti-COVID-19 antibody (e.g., an anti-SARS-CoV-2 antibody), an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. App. Pub. No. US2016/0017029 and U.S. Pat. Nos. 8,309,088 and 9,353,176) and an anti-Activin A antibody. In some aspects, the bispecific antibody is selected from the group consisting of an anti-CD3×anti-CD20 bispecific antibody (as described in U.S. Pat. Nos. 9,657,102 and 10,550,193), an anti-CD3 x anti-Mucin 16 bispecific antibody (e.g., an anti-CD3 x anti-Muc16 bispecific antibody), and an anti-CD3 x anti-Prostate-specific membrane antigen bispecific antibody (e.g., an anti-CD3 x anti-PSMA bispecific antibody).

Also included are a Met x Met antibody, an agonist antibody to NPR1, an LEPR agonist antibody, a BCMA x CD3 antibody, a MUC16 x CD28 antibody, a GITR antibody, an IL-2Rg antibody, an EGFR x CD28 antibody, an anti-PSMA x anti-CD28 bispecific antibody, antibodies against SARS-COV-2 variants, a Fel d 1 multi-antibody therapy, a Bet v 1 multi-antibody therapy, an anti-PDL-1 antibody, an anti-PD-L1 x anti-IL2Ra antibody, an anti-Dll4 antibody, a PDGF-b antagonist, an anti-NPR1 antibody, an anti-Factor XII antibody, an anti-TMPRSS6 antibody, an anti-Factor XI antibody, a bispecific anti-PSMA, an anti-PD1-IL2Ra antibody, a bispecific anti-CD38 and anti-CD28 antibody, a bispecific anti-CD22 and anti-CD28 antibody, and a bispecific anti-MUC16 and anti-CD28 antibody. Additional exemplary bispecific antibodies and bispecific preparations include Dupilumab/Linvoseltamab (IL-4R/BCMA and CD3).

Dupilumab (Dupixent®) is a monoclonal antibody developed as a collaboration between Regeneron and Sanofi, approved in the United States by the Food and Drug Administration in March 2017 as the first antibody-based treatment of atopic dermatitis in adults (Thibodeaux et al., 2019, Hum. Vaccines & Immunother., 15:2129-2139; Rodrigues et al., 2019, G. Ital. Dermatol. Venereol., 154). Atopic dermatitis (AD) is a chronic inflammatory skin disorder affecting up to 20% of the worldwide population, characterized by xerotic, erythematous, and lichenified papules and plaques. Dupilumab can be used along with topical corticosteroids, or as the sole treatment (D'Ippolito and Pisano, 2018, Pharmacy and Therapeutics, 43(9):532).

In October 2018, Dupixent® was approved for the treatment of moderate-to-severe asthma with the eosinophilic phenotype for patients aged 12 and older as an add-on maintenance therapy aimed to suppress the atopic conditions and improve patient life quality by reducing symptoms and morbidity (Thibodeaux et al.). Dupixent® has also been approved, for example, for the treatment AD in children over 6 months old, asthma of the eosinophilic phenotype or when oral corticosteroid-dependent in children over 6 years old, eosinophilic esophagitis (EoE) in patients over the age of 12, prurigo nodularis (PN) in adults, and as an add-on treatment for chronic rhinosinusitis with nasal polyposis (CRSwNP) in adults (Patient Information—Dupixent® (Dupilumab) injection for subcutaneous use, Regeneron, regeneron.com/downloads/dupixent_ppi.pdf, (accessed 25 May 2023); Take Action with DUPIXENT® (Dupilumab), dupixent.com, (accessed 24 Jul. 2023)).

Dupilumab is a fully human IgG4 monoclonal antibody with a molecular weight of approximately 147 kDa and is produced using Chinese hamster ovary (CHO) cell suspension culture (D'Ippolito and Pisano; Patient Information). The antibody binds to the IL-4Ra subunits of Type 1 and Type 2 IL-4 receptors, inhibiting the IL-4 and IL-13 signaling pathways. This reduces the release of cytokines and chemokines, which are inflammatory mediators, as well as the release of nitric oxide and IgE, and leads to an increase in IL-4 and IL-13 serum levels. IL-4 and IL-13 play a key role in type 2 inflammation, which is integral to atopic diseases such as asthma, atopic dermatitis, or chronic sinusitis with nasal polyposis. IL-4 induces naïve CD4+ T cells to differentiate into Th2 effector cells, and IL-13 is involved in goblet cell metaplasia, smooth muscle alterations, fibrosis, mucus hypersecretion, and increased airway hyperreactivity. Additionally, both IL-4 and IL-13 promote the chemotaxis of eosinophils to inflammation sites, and class switching of B-cell immunoglobulins to IgE and IgG4 (in humans) or IgG1 (in mice) (Thibodeaux et al.; Le Floc'h et al., 2020, Allergy, 75:1188-1204).

The inventions are also amenable to the production of other molecules, including fusion proteins. Preferred fusion proteins include Receptor-Fc-fusion proteins, which are also referred to as “traps,” “trap molecules” or “trap proteins.” In some aspects, the protein of interest is a recombinant protein that contains an Fc moiety and another domain (e.g., an Fc-fusion protein). The Fc-fusion protein can be a receptor Fc-fusion protein, which contains one or more extracellular domain(s) of a receptor coupled to an Fc moiety. The Fc moiety can comprise a hinge region followed by a CH2 and CH3 domain of an IgG. The receptor Fc-fusion protein can contain two or more distinct receptor chains that bind to either a single ligand or multiple ligands. For example, such trap proteins include an IL-1 trap (e.g., rilonacept, which contains the IL-1RAcP ligand binding region fused to the Il-1R1 extracellular region fused to Fc of hIgG1; see U.S. Pat. No. 6,927,044), or a VEGF trap (e.g., aflibercept (including HD and ziv-aflibercept), which contains the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). The Fc-fusion protein can be a ScFv-Fc-fusion protein, which contains one or more of one or more antigen binding domain(s), such as a variable heavy chain fragment and a variable light chain fragment, of an antibody coupled to an Fc moiety.

Other proteins lacking Fc portions, such as recombinantly produced enzymes and mini-traps, also can be made according to the inventions. Mini-traps are trap proteins that use a multimerizing component (MC) instead of an Fc portion, and are disclosed in U.S. Pat. Nos. 7,279,159 and 7,087,411.

Other antibodies and targets that can be produced according to the inventions include palivizumab (RSV); daclizumab (IL-2); gemtuzumab (CD33); natalizumab (VLA-4); teplizumab (CD3); epratuzumab (CD22); briakinumab (IL-12, 23); HuM291 (CD3 fc receptor); HeFi-1 (CD30); MDX-060 (CD30); MDX-1401 (CD30); SGN-30 (CD30); HCD122 (CD40); SGN-40 (CD40); MDX-1411 (CD70); hLL1 (EPB-1) (CD74.38); MT293 (TRC093/D93) (cleaved collagen); HuLuc63 (CS1); AMG-655 (DR5); CS-1008 (DR5); IMC-11F8 (EGFR); CDX-110 (EGFRvIII); MORAb-003 (folate receptor a); KW-2871 (ganglioside GD3); MORAb-009 (GP-9); CDX-1307 (MDX-1307) (hCGb); AMG-479 (IGF-1R); anti-IGF-1R R1507 (IGF1-R); CP 751871 (IGF1-R); IMC-A12 (IGF1-R); BIIB022 (IGF-1R); Mik-beta-1 (IL-2Rb (CD122)); CNTO 328 (IL6); Anti-KIR (1-7F9); Hu3S193 (Lewis (y)); hCBE-11 (LTOR); HuHMFG1 (MUC1); RAV12 (N-linked carbohydrate epitope); CAL (parathyroid hormone-related protein (PTH-rP)); CT-011 (PD1); MDX-1106 (ono-4538) (PD1); MAb CT-011 (PD1); IMC-3G3 (PDGFRa); huJ591 (PSMA); muJ591 (PSMA); GC1008 (TGFb (pan) inhibitor (IgG4)); A27.15 (transferrin receptor); E2.3 (transferrin receptor); HuMV833 (VEGF); IMC-18F1 (VEGFR1); IMC-1121 (VEGFR2); GPC3 monoclonal antibody; AER-001; ABT-308 (also referred to as humanized 13C5.5 antibody); RG7636 (anti-ETBR); RG7458 (anti-MUC16); RG7599 (anti-NaPi2b); MPDL3280A (anti-PD-L1); RG7450 (anti-STEAP1); and GDC-0199 (anti-Bcl-2).

Other antibodies or tumor target binding proteins that can be produced according to the inventions include those that bind the following antigens (the cancer indications represent non-limiting examples): aminopeptidase N (CD13), annexin A1, CA125 (ovarian cancers), CA15-3 (carcinomas), CA19-9 (carcinomas), L6 (carcinomas), Lewis Y (carcinomas), Lewis X (carcinomas), alpha fetoprotein (carcinomas), CA242 (colorectal cancers), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma, B-cell neoplasms, autoimmune diseases), CD21 (B-cell lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (metastatic melanoma, sarcoma), CD52 (leukemia), CD56 (small cell lung cancers, ovarian cancer, Merkel cell carcinoma, and the liquid tumor, multiple myeloma), CD66e (carcinomas), CD70 (metastatic renal cell carcinoma and non-Hodgkin's lymphoma), CD74 (multiple myeloma), CD80 (lymphoma), CD98 (carcinomas), CD123 (leukemia), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (CEA, CD66e) (breast, colorectal and lung cancers), DLL4 (A-like-4), EGFR (various cancers), CTLA4 (melanoma), CXCR4 (CD 184, heme-oncology, solid tumors), Endoglin (CD 105, solid tumors), EPCAM (epithelial cell adhesion molecule, bladder, head, neck, colon, NHL prostate, and ovarian cancers), ERBB2 (lung, breast, prostate cancers), FCGR1 (autoimmune diseases), FOLR (folate receptor, ovarian cancers), FGFR (carcinomas), GD2 ganglioside (carcinomas), G-28 (a cell surface antigen glycolipid, melanoma), GD3 idiotype (carcinomas), heat shock proteins (carcinomas), HER1 (lung, stomach cancers), HER2 (breast, lung and ovarian cancers), HLA-DR10 (NHL), HLA-DRB (NHL, B cell leukemia), human chorionic gonadotropin (carcinomas), IGF1R (solid tumors, blood cancers), IL-2 receptor (T-cell leukemia and lymphomas), IL-6R (multiple myeloma, RA, Castleman's disease, IL6 dependent tumors), integrins (avP3, α5β1, 0604, al 103, 0505, avP5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1) (Non-Hodgkin's B cell lymphoma, leukemia), MUC1 (breast, ovarian, cervix, bronchus and gastrointestinal cancer), MUC16 (CA125) (ovarian cancers), CEA (colorectal cancer), gp100 (melanoma), MARTI (melanoma), MPG (melanoma), MS4A1 (membrane-spanning 4-domains subfamily A) (small cell lung cancers, NHL), nucleolin, Neu oncogene product (carcinomas), P21 (carcinomas), nectin-4 (carcinomas), paratope of anti-N-glycolylneuraminic acid (breast, melanoma cancers), PLAP-like testicular alkaline phosphatase (ovarian, testicular cancers), PSMA (prostate tumors), PSA (prostate), ROB04, TAG 72 (tumour associated glycoprotein 72) (AML, gastric, colorectal, ovarian cancers), T cell transmembrane protein (cancers), Tie (CD202b), tissue factor, TNFRSF10B (tumor necrosis factor receptor superfamily member 10B) (carcinomas), TNFRSF13B (tumor necrosis factor receptor superfamily member 13B) (multiple myeloma, NHL, other cancers, RA and SLE), TPBG (trophoblast glycoprotein) (renal cell carcinoma), TRAIL-R1 (tumor necrosis apoptosis inducing ligand receptor 1) (lymphoma, NHL, colorectal, lung cancers), VCAM-1 (CD106, Melanoma), VEGF, VEGF-A, and VEGF-2 (CD309) (various cancers).

Derivatives, components, domains, chains and fragments of the above also are included. In one aspect, the protein of interest or polypeptide of interest comprises a combination of any of the foregoing.

In addition to next generation products, the inventions also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product that is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines also are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to a reference product already approved in Japan. In India, biosimilars currently are referred to as “similar biologics,” and refer to a similar biologic product which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, a biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be undertaken according to the inventions.

In some exemplary aspects, cells of the present invention can be mammalian cells. The mammalian cells can be of human origin or non-human origin, and can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK (15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

In some aspects, cells that are useful in the method of the present invention are selected from the group consisting of CHO cells, HEK293 cells, and BHK cells. In other aspects, the cells are selected from the group consisting of CHO-K1, CHO DUX B-11, Veggie-CHO, GS-CHO, S-CHO, or CHO lec. In some exemplary aspects, the cells of the present invention are CHO-K1 cells.

Manufacture of proteins via cell culture is customarily performed using a batch or fed-batch process. Early stages of inoculum growth after vial thaw include culturing cells in a seed culture. Culturing vessels include, but are not limited to, well plates, wave bags, T-flasks, shake flasks, stirred vessels, spinner flasks, hollow fiber, air lift bioreactors, and the like. A suitable cell culturing vessel is a bioreactor. A bioreactor refers to any culturing vessel that is manufactured or engineered to manipulate or control environmental conditions. Such culturing vessels are well known in the art. Typically, cells are grown at an exponential growth rate, such as in seed train bioreactors, in order to progressively increase size and/or volume of the cell population. Seed train vessels may be referred to in relation to their order before a production bioreactor; for example, the final seed train vessel may be referred to as N-1, the preceding vessel as N-2, and so forth. After cell mass is scaled up through several bioreactor stages, cells are then transferred to a production bioreactor while the cells are still in exponential growth (log phase). It is generally considered undesirable to allow cells in batch culture, for example seed culture, to go past the log phase into stationary phase. It has been recommended that cultures should be passaged while they are in log phase, before, cells, e.g. adherent cells, reach confluence due to contact inhibition or accumulation of waste products inhibits cell growth, among other reasons (Cell Culture Basics, Gibco/Invitrogen Online Handbook, www.invitrogen.com; ATCC® Animal Cell Culture Guide, atcc.org).

The terms “early phase,” “middle phase,” and “late phase,” as used herein, refer to time periods in a cell culture after inoculation which may be biologically distinct, and are easily understood by a skilled person. In some aspects, an early phase corresponds approximately or precisely to a logarithmic growth phase (or exponential growth phase), which may be determined by an increasing slope of a cell proliferation curve as measured by, for example, viable cell density. In some aspects, a middle phase corresponds approximately or precisely to a stationary phase, which may be distinguished from an early phase by a decreasing slope of a cell proliferation curve. In some aspects, a late phase corresponds approximately or precisely to a death phase (or decline phase), which may be distinguished from a middle phase by a negative slope of a cell proliferation curve. Determination of early, middle, and late phases may be made based on historical understanding of average cell growth curves when using a known manufacturing process, and does not need to be empirically determined for each cell culture batch or group of cell culture batches.

In some aspects, an early phase excursion may begin from 6 to 60, from 12 to 48, from 24 to 30, about 6, about 12, about 18, about 24, about 30, about 36, about 42, about 48, about 54, or about 60 hours after inoculation. In specific aspects, an early phase excursion may begin about 24 to 30 hours after inoculation. In some aspects, a middle phase excursion may begin from 78 to 138, from 84 to 120, from 96 to 102, about 78, about 84, about 90, about 96, about 102, about 108, about 114, about 120, about 126, about 132, or about 138 hours after inoculation. In specific aspects, a middle phase excursion may begin about 96 to 102 hours after inoculation. In some aspects, a late phase excursion may begin from 180 to 270, from 192 to 252, from 204 to 240, from 216 to 222, about 180, about 186, about 192, about 198, about 204, about 210, about 216, about 222, about 228, about 234, about 240, about 246, about 252, about 258, about 264, or about 270 hours after inoculation. In specific aspects, a late phase excursion may begin about 216 to 222 hours after inoculation.

Following protein expression in a production bioreactor, recombinant proteins may be harvested and subjected to further downstream processing steps in order to prepare the protein for analysis and/or for use as a product. Prior to harvesting, cell cultures may be subjected to pre-treatment steps to induce flocculation or precipitation of undesired elements, for example using a modified temperature, pH, or additive. Harvesting may include, for example, separating proteins from cells and debris using centrifugation and/or filtration. Harvesting may take place at, for example, about day 10 in the production bioreactor, about day 11, about day 12, about day 13, about day 14, or about day 15.

Harvested samples may then be subjected to affinity chromatography to enrich a recombinant protein of interest. Protein A affinity chromatography may be particularly useful for enriching recombinant antibodies, antibody fragments, antibody fusion proteins or proteins otherwise derived from antibodies. Following affinity chromatography, enriched samples may be transiently subjected to an acidic pH for viral inactivation. Enriched samples may be subjected to further chromatographic processes, including, for example, reverse phase liquid chromatography, ion exchange chromatography (for example, anion exchange chromatography and/or cation exchange chromatography), hydrophobic interaction chromatography, hydrophilic interaction chromatography, size exclusion chromatography, and/or mixed-mode chromatography. Enriched protein samples following chromatographic steps may be further subjected to, for example, virus retentive filtration, ultrafiltration, diafiltration, concentration, buffer exchange, and/or formulation with excipients. It should be understood that these steps may be customized, modified, omitted, expanded upon, or rearranged by a skilled person to accommodate a particular process and protein of interest, based on common knowledge in the field.

In some exemplary aspects, the invention can include characterizing and/or predicting product quality attributes (PQA) and/or critical quality attributes (CQA). Identifying critical Product Quality (cPQ) attributes of a biopharmaceutical is key to ensuring the efficacy and safety of the product. According to the ICH Q8 (R2) Scientific Guideline for Pharmaceutical Development, a critical quality attribute is “a physical, chemical, biological, or microbiological property or characteristic that should be within an appropriate limit, range, or distribution to ensure the desired product quality” (ICH guideline Q8 (R2) on pharmaceutical development, European Medicines Agency, ema.europa.eu/en/documents/scientific-guideline/international-conference-harmonisation-technical-requirements-registration-pharmaceuticals-human-use_en-11.pdf (accessed 16 Jun. 2023)). Understanding the cPQ attributes and analyzing their variability is necessary to define the acceptance criteria and the quality target product profile (QTPP) (Alt et al., 2016, Biologicals, 44:291-305), and understanding how the various process parameters affect cPQ allows the development of the product with a quality by design (QbD) approach (Reusch and Tejada, 2015, Glycobiology, 25:1325-1334).

cPQ attributes can affect the product purity, stability, strength, and drug release, as well as other aspects specific to the formulation type, such as adhesion properties of patches, the sterility of products administered parenterally, or the aerodynamic properties of inhaled drugs (ICH guideline).

Assessing the cPQ includes detecting and quantifying not only process-related impurities but also aggregates, fragments, and product variants, commonly including charge variants (basic or acidic), size variants, oxidation-related variants, structural variants, and variants arising from glycosylation of the Fc region of antibodies (Alt et al.). Examples of such monoclonal antibody variants are listed in the table below.

| Variant type | Example |
|---|---|
| Charge (basic) | Species with isomerized aspartic acid in |
| | complementarity determining regions |
| Charge (acidic) | Deamidated species |
| Size | HMW species; LMW species |
| Oxidation-related | Species with oxidized methionine |
| Structural | Sequence variants |
| Glycosylation-related | Afucosylated species |

A risk assessment can be performed to analyze the cPQ attributes and process parameters in terms of their impact on safety, pharmacokinetics (PK), bioactivity, and immunogenicity. This is usually done at an early stage of the development process based on initial experiments and prior knowledge, and the assessment is refined at a later stage, incorporating more experimental data and mathematical models (ICH guideline; Alt et al.). Impact scores may be used to better define vague terms such as “high impact” or “low impact” using point scales (Alt et al.). Non-limiting of examples of assays suitable for assessing product quality attributes include chromatography (including RPLC, IEX, AEX, CEX, SEC, HIC, HILIC, and MMC), mass spectrometry (including intact mass analysis, peptide mapping, and amino acid sequencing), spectroscopy (including UV/vis spectroscopy), capillary electrophoresis (including free flow electrophoresis, isoelectric focusing, capillary isoelectric focusing, imaged capillary isoelectric focusing, and capillary zone electrophoresis), and gel electrophoresis (including SDS-PAGE and western blotting), and ligand binding assays (including biolayer interferometry, enzyme-linked immunosorbent assay, and surface plasmon resonance).

In some aspects, the invention includes assessment of charge variants of a protein, for example using imaged capillary isoelectric focusing (iCIEF) or another electrophoretic process. Charge variants may be classified based on detectable signal in a defined region of an electropherogram or other output of a process for separating protein variants by charge. In some aspects, charge variants may be classified as iCIEF Region 1, iCIEF Region 2, and iCIEF Region 3. Each classification of charge variant may have a distinct upper and lower performance target. In particular, iCIEF Region 1 and iCIEF Region 3 may represent charge variants with an upper limit as a performance target while iCIEF Region 2 represents charge variants (or main charge species) with a lower limit as a performance target. Alternatively, any of the classifications may have a two-sided performance target, with an acceptable range falling between an upper limit and a lower limit.

As used herein, the term “DOE” refers to the design of an experiment that can be facilitated by selected instrument settings, multivariate analysis, and/or computer-aided design and software. DOE allows for screening of different combinations of parameters to determine the mathematical relationship between the parameters and other outputs of interest. DOE is often used to find the combinations of parameter settings that provide optimal response of the desired output. Rather than evaluating “one-factor at a time” (OFAT), which may be inefficient considering the number of possible experimental and instrument parameters, DOE allows for simultaneous evaluation of an experiment or instrument's many different parameters.

In some aspects, the invention includes analysis of gene expression, for example, identification and quantification of mRNA in a cell culture sample. Suitable methods of determining expression levels of mRNA are known in the art and include, for example, quantitative polymerase chain reaction (qPCR) (e.g., quantitative real-time PCR (qRT-PCR)), RNAseq, Northern blotting, serial analysis of gene expression (SAGE), the MassARRAY system, in situ hybridization (ISH), and combinations thereof. Techniques for measuring gene expression also include, for example, gene expression assays with or without the use of gene chips or gene expression microarrays. Affymetrix gene chips and RNA chips and gene expression assay kits are also commercially available.

In some aspects, the invention includes analysis of cell culture parameters using an analyzer, for example a NOVA BioProfile® Flex Analyzer. Any analyzer capable of quantifying cell viability, cell density, pH, temperature, dissolved gas, and/or metabolite concentrations may be suitable for use in the invention. Samples for analysis may be obtained for inline, online, or offline analysis. In some aspects, a port in a bioreactor is coupled online to an analyzer and allows for continuous analysis of cell culture conditions.

As used herein, the term “biomarker” refers to a measurable indicator of a biological state. In particular, biomarkers of the present invention include components or properties of a cell culture that are useful for predicting, for example, cell culture health or recombinant protein properties. In one aspect, a biomarker is a gene, or a corresponding nucleic acid molecule or gene product, that is differentially expressed between cell culture batches with different cell culture outcomes or recombinant protein outcomes. In one aspect, a biomarker may be a metabolite concentration or other property of a cell culture that may be affected downstream of any change in gene expression.

For example, a biomarker may be a gene that has a statistically significantly higher or lower level of expression in a cell culture that will produce a recombinant protein that will pass product quality testing compared to in a cell culture that will produce a recombinant protein that will fail product quality testing. A biomarker useful in the invention may be a hypoxia-related gene or may be in an unrelated molecular pathway. This disclosure sets forth methods and systems for identifying useful biomarkers in a cell culture process, for using measurements of biomarkers to predict the outcome of a cell culture process, for example with a regression model, and for using predictions based on biomarkers to modify the cell culture process for an improved manufacturing process.

It is understood that the present invention is not limited to any of the aforesaid input parameter(s), output parameter(s), protein(s), antibody(s), monoclonal antibody(s), bispecific antibody(s), protein expression system(s), multisubunit protein(s), cell(s), vessel(s), excursion(s), model(s), analyzer(s), or cell culture condition(s), and any input parameter(s), output parameter(s), protein(s), antibody(s), monoclonal antibody(s), bispecific antibody(s), protein expression system(s), multisubunit protein(s), cell(s), vessel(s), excursion(s), model(s), analyzer(s), or cell culture condition(s) can be selected by any suitable means.

The present invention will be more fully understood by reference to the following Examples. They should not, however, be construed as limiting the scope of the invention.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

### Example 1. Dissolved Oxygen Excursion Design of Experiments for mAb1

### 1.1. Study Parameters

Several input parameters relevant to DO excursions were considered for inclusion in a laboratory-scale design of experiments screening. Commonly experienced DO excursions were reviewed, as well as those that may have the most impact to a batch (long excursions) versus the most impact to a compliance system (short or oscillatory excursions). Table 1 summarizes the input parameters selected to characterize the impacts that DO excursions have to product quality and cellular health, and the rationale for inclusion.

TABLE 1
DO excursion input parameters

| Input | | |
|---|---|---|
| Parameter | Possible Impact | Rationale |
| Duration | Fluctuations could cause | Provides insight into how |
| of DO | changes in cellular | long a DO excursion |
| excursion | metabolism, cell growth | can last before it impacts |
| | and/or cell death. | the cell culture to a |
| | | point at which product |
| | | quality is affected. |
| Timing | Phase of bioreactor when | Can elucidate which |
| of DO | the DO excursion occurs | phases of production (lag, |
| excursion | could impact cellular | log, stationary) are most |
| | metabolism, cell growth | sensitive to low DO |
| | and/or cell death, | excursions. Certain phases |
| | and affect titer and | of the bioreactor |
| | product quality. | may be more sensitive |
| | | to a DO excursion. |
| Magnitude | Variations could cause | Can help determine how |
| of DO | differential changes in | low the DO must drop |
| excursion | cellular metabolism, cell | before a statistically |
| | growth and/or cell death, | significant change in |
| | and may differentially | product quality is |
| | affect product quality. | observed. |

Table 2 summarizes output parameters selected to assess process performance. The relative importance of each parameter for determining process consistency, product safety and product efficacy was considered for each parameter. The assessment of these outputs provides the basis for determining acceptable DO excursions that may not need to be escalated in the quality system and may not result in a product that fails to meet performance targets or specification limits.

TABLE 2
Selected output parameters to assess effects of DO excursions

| Selected Output Parameter | Sample Point |
|---|---|
| Final cell culture viability (%) | Production bioreactor, final |
| Final titer (g/L) | sample before harvest |
| Purity, NGHC, and LMW species by reduced MCE (%) | Viral inactivated (VI) pool |
| Purity, LMW, and HMW species by non-reduced MCE (%) | |
| Main peak purity and HMW by SE-UPLC (%) | |
| Charge Variant Profile (% Regions 1, 2, and 3) | |
| Glycosylation Profile (% total fucosylation, G0 + G0F, | |
| G1 + G1F, G2 + G2F, Man-5) | |
| RNA expression profiles | Daily samples during |
| | production, enhanced sampling |
| Protein expression profiles | post-excursion |
| DO traces | Daily throughout production |
| | for information only |

The experimental design of the study was created by applying design of experiment (DOE) techniques using Stat-Ease's DOE software, Design-Expert. Table 3 summarizes the settings selected for each input parameter included in this study.

TABLE 3
Study parameters

| | Nominal | Lowest | Highest |
|---|---|---|---|
| Parameter | Conditions | Setting | Setting |
| Duration of DO | 0 | 24 (per phase) | 96 (per phase) |
| excursion (hours) | | | |
| Timing of DO | No | Excursion | Excursion in |
| excursion | excursions. | only occurs | all phases. |
| (process day) | | in one phase. | |
| DO setpoint during | 40.4 | 0.0 | 8.0 |
| excursion (%) | | | |

The study was designed to consist of thirty-six 2 L production bioreactor batches, run in three blocks of twelve bioreactors each. Each block contained two control bioreactor batches run at the manufacturing operating setpoint for the entire batch duration. The block assignments and study parameter settings for each batch are summarized in Table 4. “Y” indicates that an excursion occurs in this phase of production for the hours listed in the corresponding excursion duration; “N” indicates that no excursion occurs in this phase of production. For excursions greater than 72 hours in duration that occur in sequential phases, the excursion was continued for the total excursion length even though the start time for the second excursion occurs outside the defined target window. The DO setting is described in Table 4 in terms of absolute DO %, but it should be understood that a magnitude of an excursion can also be expressed relative to nominal conditions. For example, given nominal conditions of a DO setpoint of about 40% DO, a DO excursion with 8% DO could alternatively be expressed as an excursion with a relative DO of about 20% of the DO setpoint.

TABLE 4
Study design

| | | | | Excursion in | Excursion in | Excursion in | Total |
|---|---|---|---|---|---|---|---|
| | | DO | Excursion | Early Phase | Middle Phase | Late Phase | Excursion |
| Run | | Setting | Duration | (24-30 hours | (96-102 hours | (216-222 hours | Length |
| ID | Block | (%) | (hours) | post-inoculation) | post-inoculation) | post-inoculation) | (hours) |
| 1 | 1 | 7.0 | 69 | Y | N | Y | 138 |
| 2 | | 8.0 | 24 | Y | Y | Y | 72 |
| 3 | | 0.0 | 52 | N | Y | N | 52 |
| 4 | | 8.0 | 24 | Y | Y | N | 48 |
| 5 | | 0.0 | 96 | Y | Y | N | 192 |
| 6 | | 4.0 | 60 | Y | Y | Y | 180 |
| 7 | | 5.0 | 96 | N | Y | N | 96 |
| 8 | | 8.0 | 24 | Y | Y | N | 48 |
| 9 | | 0.0 | 24 | N | N | Y | 24 |
| 10 | | 5.6 | 76 | N | Y | Y | 152 |
| 11 | | 40.4 | 0 | N | N | N | 0 |
| 12 | | 40.4 | 0 | N | N | N | 0 |
| 13 | 2 | 0.0 | 24 | N | N | Y | 24 |
| 14 | | 4.0 | 60 | N | N | Y | 60 |
| 15 | | 2.9 | 24 | N | Y | N | 24 |
| 16 | | 8.0 | 96 | Y | Y | Y | 288 |
| 17 | | 8.0 | 96 | Y | N | N | 96 |
| 18 | | 8.0 | 70 | N | Y | N | 70 |
| 19 | | 8.0 | 24 | Y | N | Y | 48 |
| 20 | | 8.0 | 96 | Y | N | N | 96 |
| 21 | | 0.0 | 24 | Y | Y | Y | 72 |
| 22 | | 0.0 | 96 | Y | Y | Y | 288 |
| 23 | | 40.4 | 0 | N | N | N | 0 |
| 24 | | 40.4 | 0 | N | N | N | 0 |
| 25 | 3 | 0.0 | 24 | Y | N | N | 24 |
| 26 | | 0.0 | 96 | Y | N | Y | 96 |
| 27 | | 0.0 | 24 | Y | N | N | 24 |
| 28 | | 5.7 | 75 | Y | Y | N | 150 |
| 29 | | 0.0 | 96 | N | Y | Y | 192 |
| 30 | | 8.0 | 42 | Y | Y | Y | 126 |
| 31 | | 8.0 | 96 | N | N | Y | 96 |
| 32 | | 0.0 | 96 | N | Y | Y | 192 |
| 33 | | 5.6 | 75 | Y | Y | Y | 225 |
| 34 | | 8.0 | 24 | N | Y | Y | 48 |
| 35 | | 40.4 | 0 | N | N | N | 0 |
| 36 | | 40.4 | 0 | N | N | N | 0 |

The sample collection and testing strategy is summarized in Table 5. Sample volumes were approximate. Multiple tests could be run from the same sample.

TABLE 5
Sampling and testing strategy

| | Sample | |
|---|---|---|
| Time of Sampling | Volume | Test |
| Production batch days 0 | 2 mL | NOVA BioProfile ® Flex Analyzer (viable cell density/ |
| through harvest | | cell culture viability/cell culture characteristics) |
| Production batch day 0 | 2 mL | Enhanced sampling (cell pellet for RNA extraction for |
| | | RNA expression data, spent media for amino acid analysis). |
| Production batch days 1 | 1 mL | Enhanced sampling (cell pellet for RNA extraction for |
| through harvest | | RNA expression data, spent media for amino acid analysis). |
| Production batch DO | 1 mL | Enhanced sampling (cell pellet for RNA extraction for |
| excursion end point | | RNA expression data, spent media for amino acid analysis). |
| Production batch days | 5 mL | Protein titer determination by process sciences HPLC |
| 4, 6, 8-11 | | |
| Production batch day | 5 mL | Protein titer determination by HPLC for early phase |
| of harvest | | clinical monoclonal antibodies |
| Post-affinity | 10 mL | N-glycan analysis by RapiFluor-MS labeling with |
| chromatography | | hydrophilic interaction chromatography |
| (Protein A-viral | | Antibody charge variant analysis by imaging capillary |
| inactivated pool) | | isoelectric focusing |
| | | Purity analysis of in process test articles by MCE |
| | | Monoclonal antibody purity by size exclusion ultra-high |
| | | performance liquid chromatography |

### 1.2. Laboratory-Scale Design of Experiments Screening

Seed expansion in the laboratory-scale system followed an expansion schedule representative of the manufacturing process. Culture volumes during shaker flask and cellbag process steps are identical between manufacturing and laboratory-scale systems. Volumes during seed expansion (N-2, N-1) and production (N) single-use bioreactor (SUB) unit operations were scaled to match ratios of cell culture to fresh medium used during the manufacturing process. Volumes of any nutrient additions within the laboratory-scale system were also scaled appropriately to match the ratio employed within the manufacturing-scale system. It should be understood that the inventions disclosed herein, including in Examples 1-5, are applicable at both laboratory-scale and manufacturing-scale.

Recombinant CHO cells expressing a recombinant IgG4 antibody, mAb1, were thawed and inoculated into a 500 mL shaker flask with CHO medium. After sufficient time for cell growth, cells were sequentially transferred to a 2 L cellbag, a 20 L cellbag, a 2 L N-2 bioreactor, a 2 L N-1 bioreactor, and finally a 2 L production bioreactor. Each production bioreactor was sampled as specified in Table 5. DO setpoints were adjusted as specified in Table 4, and then returned to the default setpoint of 40.4%. Bioreactors were sampled and analyzed as specified in Table 5 until the batch duration of about 288 hours (12 days) had elapsed, and then proceeded to harvest.

Production bioreactor material was harvested following completion of the production unit operation. Harvest set-up and pre-use filter flushes were performed in a manner representative of the manufacturing process. Volumes of any water and buffer flushes within the laboratory-scale system were scaled appropriately to match or exceed the buffer volume to membrane surface area ratio employed within the manufacturing-scale system. Briefly, cell culture samples were subjected to filtration, Protein A affinity chromatography, and viral inactivation prior to subsequent testing.

Cell pellets and spent media can be further processed for assessing gene expression levels, protein expression analysis, and metabolomics. Differences in gene expression, protein expression, and metabolite levels can be compared between production bioreactors with varying excursions to determine how DO excursions impact cellular health and metabolism. Unique molecular profiles found to be associated with particular excursion features likely precede phenotypic cellular changes, and could serve as biomarkers for understanding and predicting cell culture outcomes.

### 1.3. Impact of DO Excursions on Product Quality

Upon completion of the study outlined in Example 1.1 and 1.2, bioreactor batches were classified into categories based on final day titer and cell viability to understand and visualize initial results. The four main effect groups are described in Table 6.

TABLE 6
Effects of DO excursions by group

| Effect Group | Criteria | Batches |
|---|---|---|
| Minimal | <15% decrease in final | 2, 8, 9, 13, 15, 16, 17, |
| | day titer | 18, 19, 25, 31, 34 |
| Moderate | >15% and < 50% decrease | 1, 3, 4, 7, 14, 20, 27, 30 |
| | in final day titer | |
| Major | >50% decrease in final day | 6, 10, 21, 26, 28, 33 |
| | titer | |
| Critical/Dead | Final cell viability <1% | 5, 22, 29, 32 |

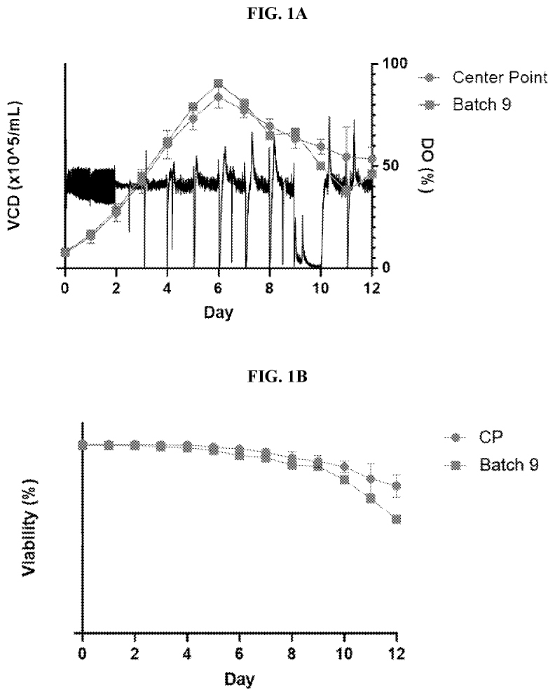

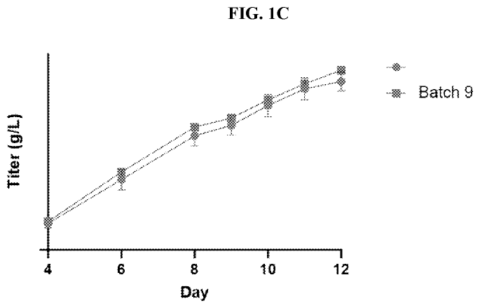

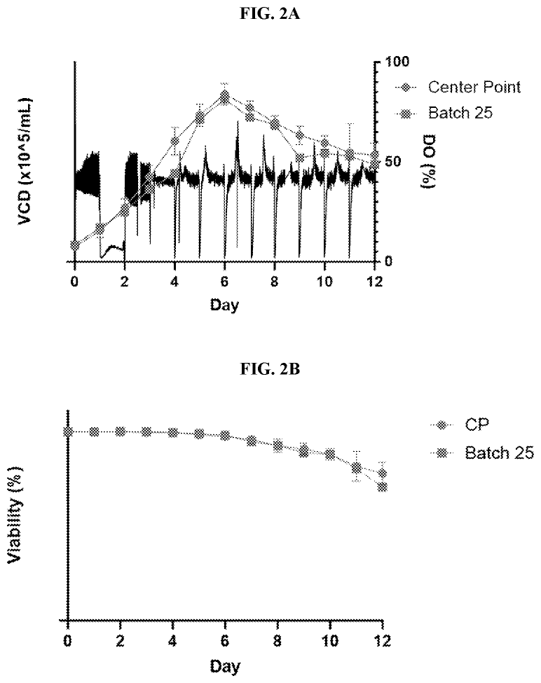

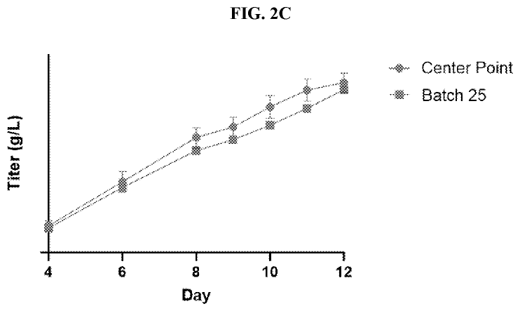

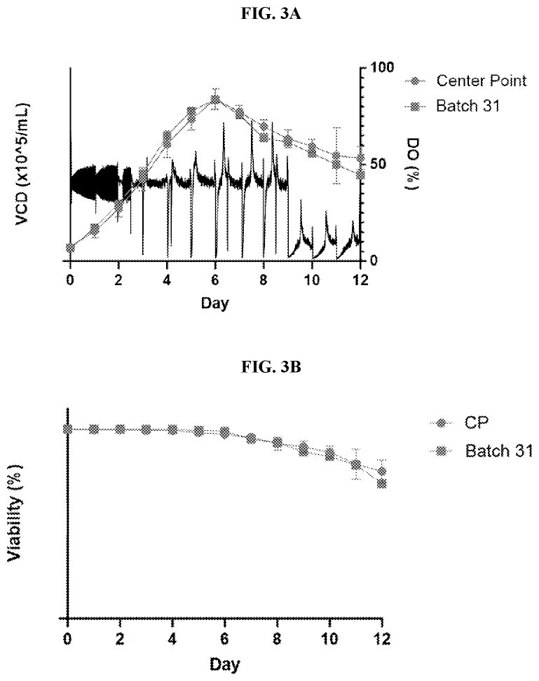

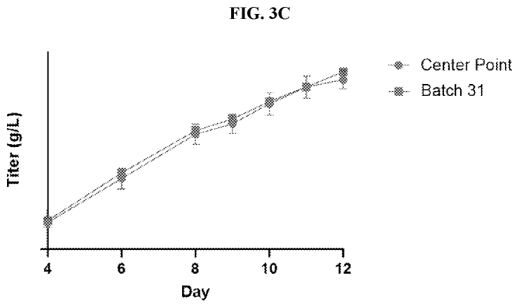

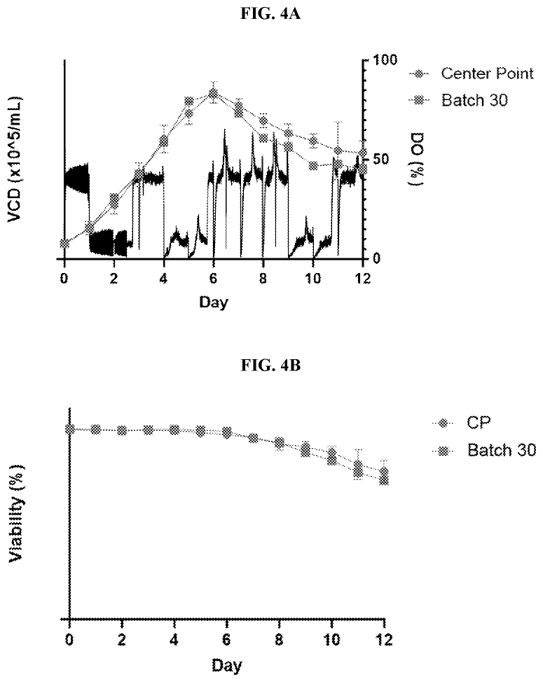

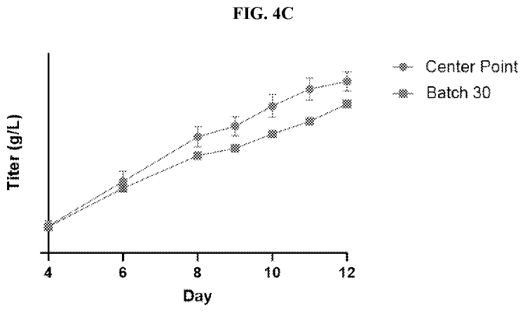

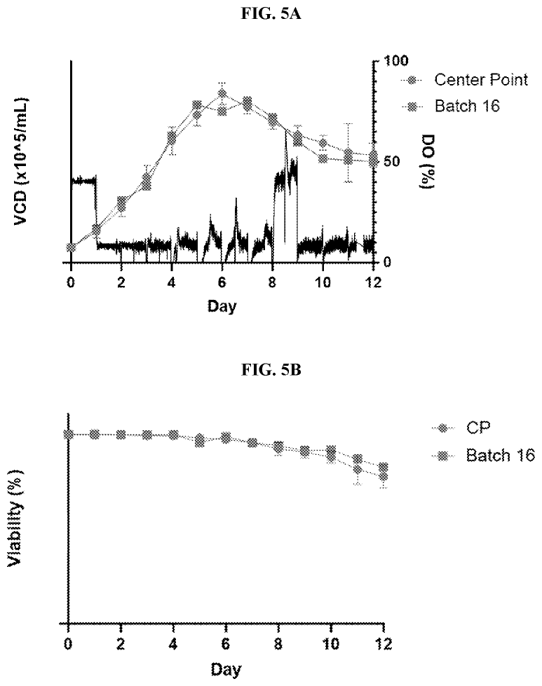

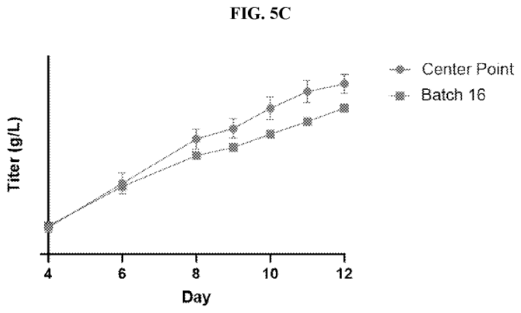

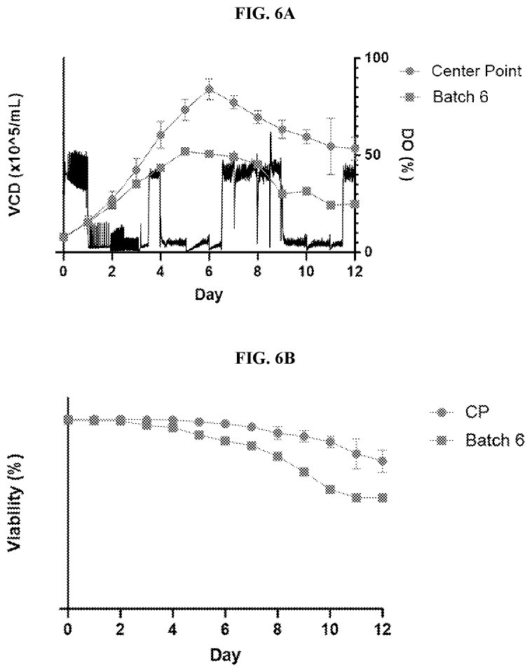

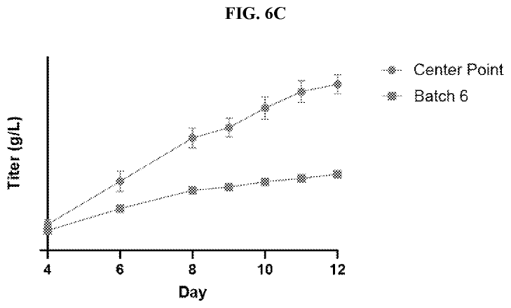

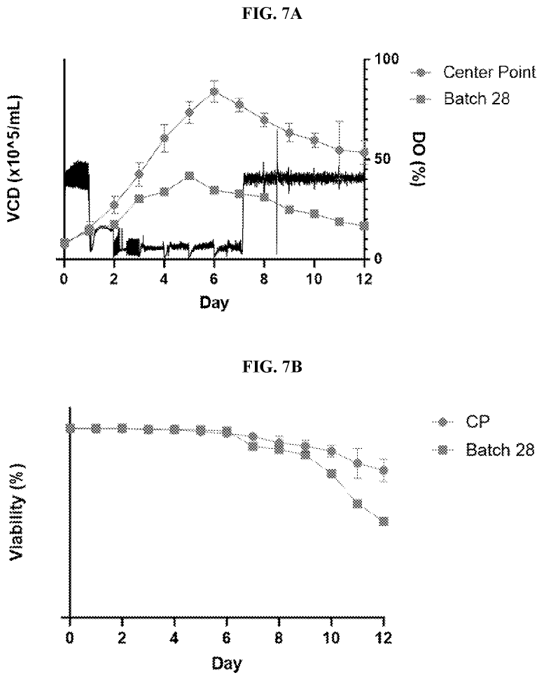

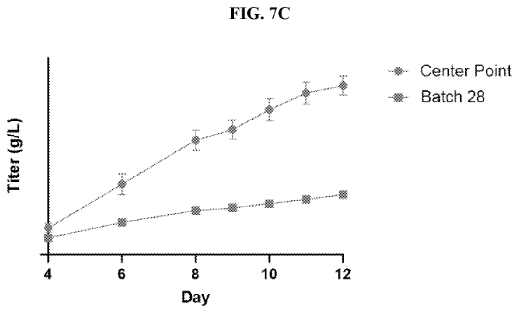

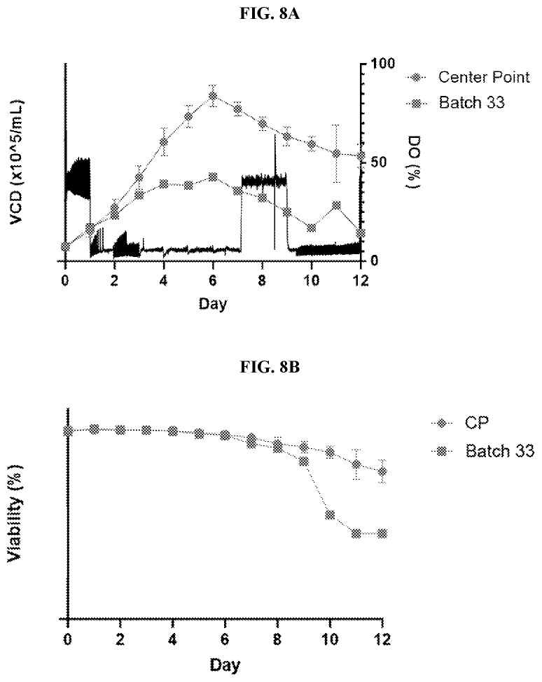

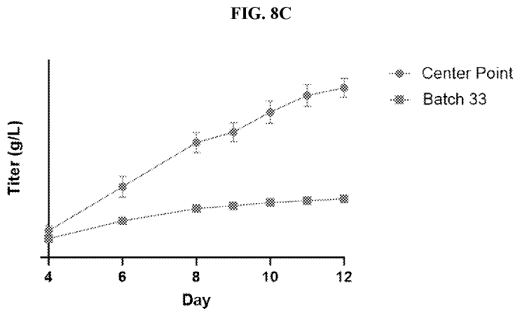

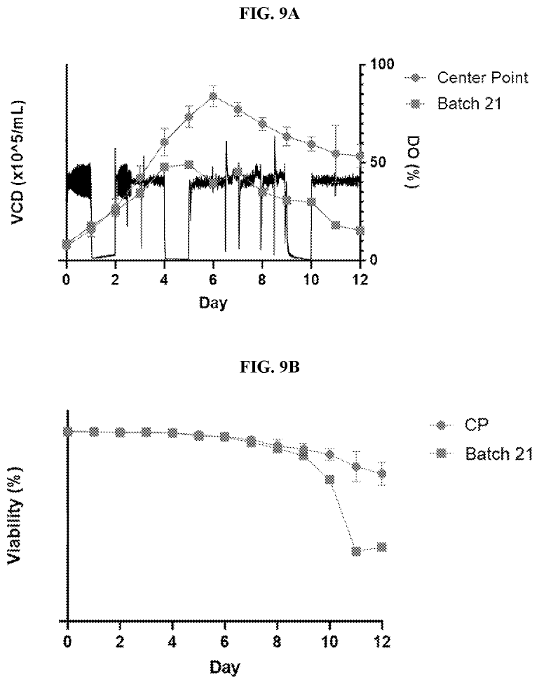

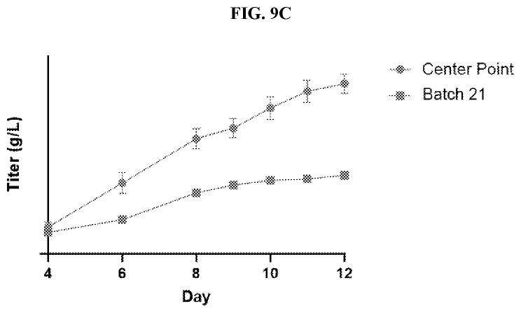

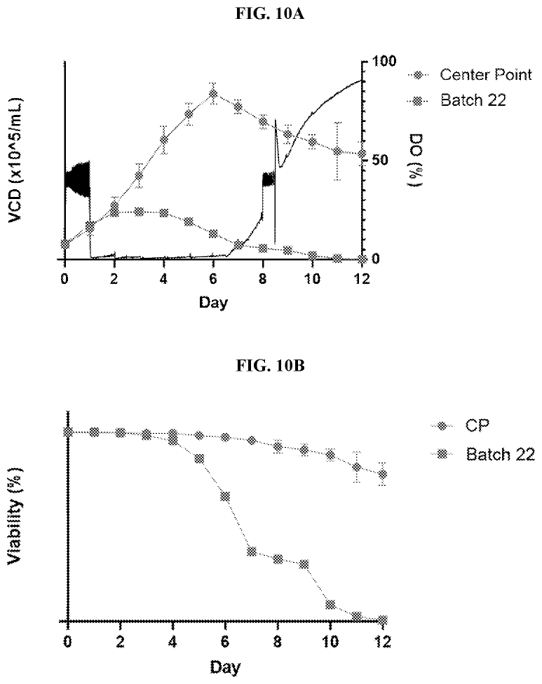

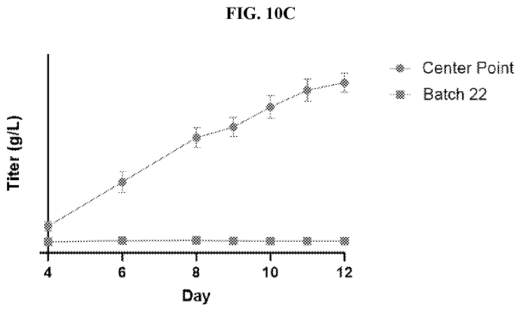

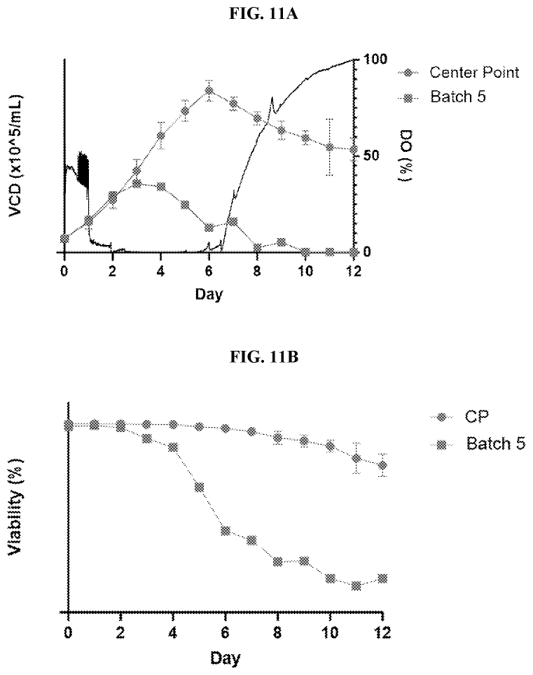

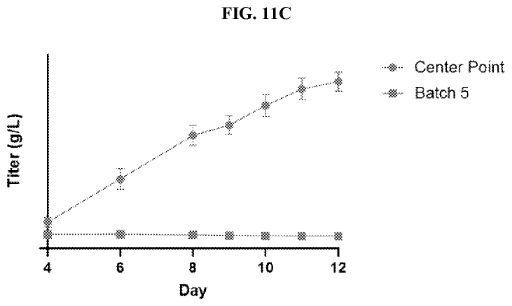

Through this sorting, it was determined that 40% of experimental batches had only a minimal decrease in final day titer, indicating that some excursions have little effect to overall cell culture health and production. Viable cell density, cell viability, and protein titer for representative batches compared to an average of control batches (center point, CP) are shown in FIGS. 1-3 (minimal effect), FIGS. 4-5 (moderate effect), FIGS. 6-9 (major effect), and FIGS. 10-11 (critical effect).

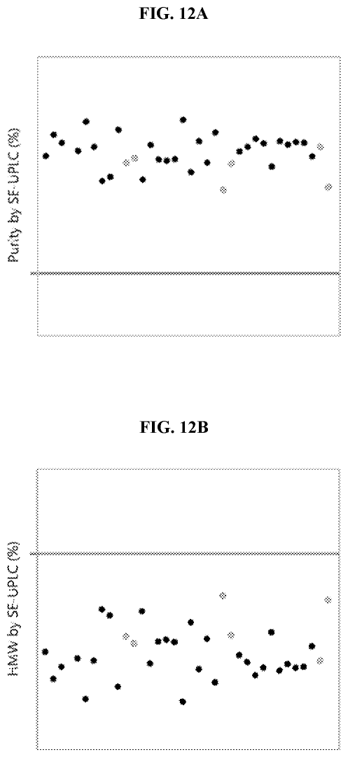

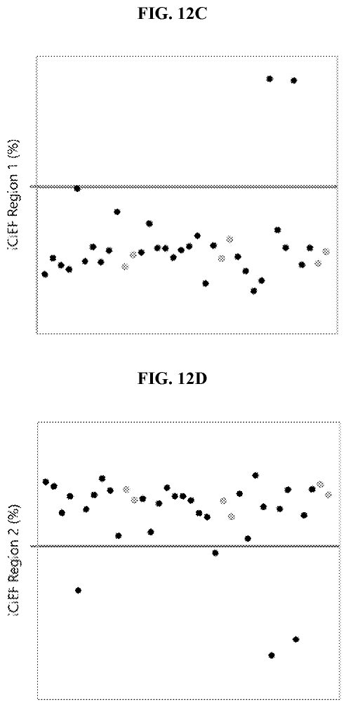

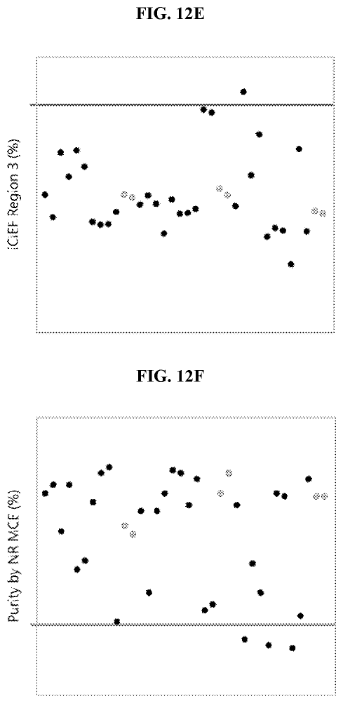

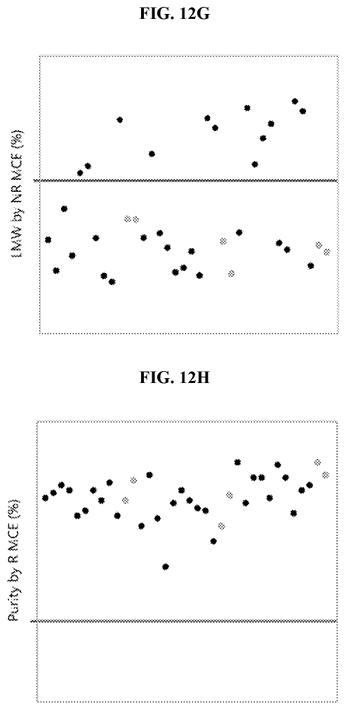

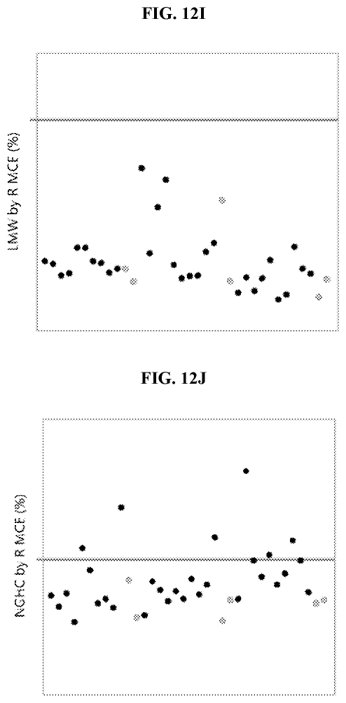

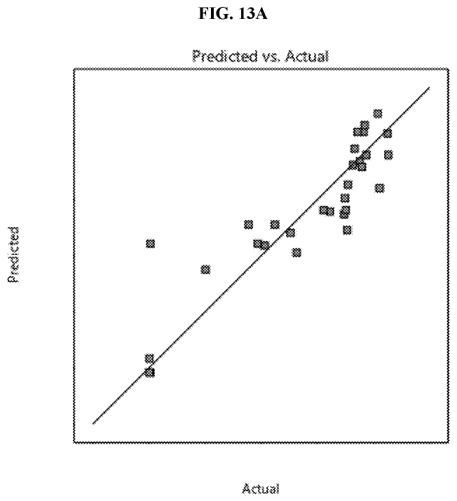

Harvested mAb1 was characterized as described in Table 5 for a variety of product quality attributes. The results for each batch and attribute are shown in FIG. 12, with control batches shown in gray.

### 1.4. Model Analysis

Half-normal factorial models were developed using analysis of variance (ANOVA) techniques to describe the influence of each studied input parameter on each selected output parameter for the mAb1 manufacturing process of Example 1.1-1.3. Significant models were determined for final viability, final titer, select glycosylation profiles, purity by reduced (R) and non-reduced (NR) MCE, LMW by NR MCE, NGHC by R MCE, and charge variants (imaged capillary isoelectric focusing (iCIEF) Regions 1, 2, and 3). No significant model was determined for HMW, LMW, and purity by SE-UPLC, select glycosylation profiles, and LMW by R MCE.

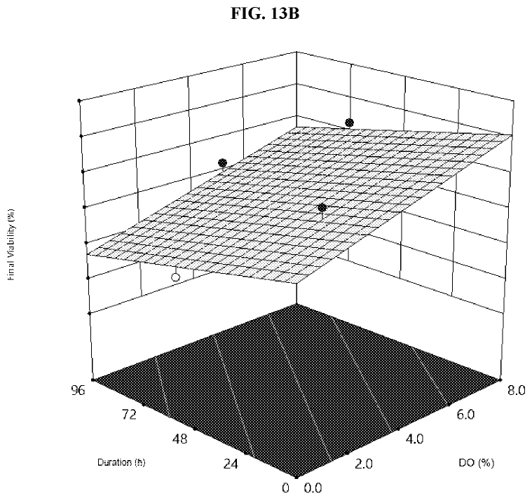

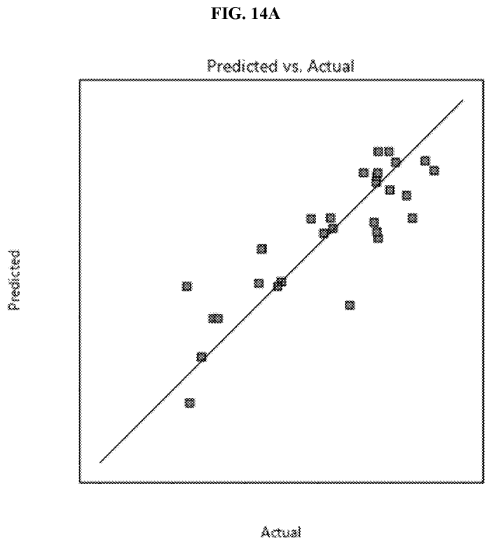

Based on the prediction models derived from the mAb1 DOE study, during the production bioreactor unit operation, cell cultures can generally survive and produce the targeted product during DO excursions, with minimal impact to product quality. Protein titer is impacted by DO excursion duration, magnitude, and phase, but minimal impact is seen for production late-stage DO excursions. Excursions to 8% DO had little effect on cell growth, viability, or titer. Excursions to 0% to 5.6% for longer periods of time had significant impacts on VCD, viability, and titer. Excursions to 0% DO were well tolerated for up to 24 hours, but multiple 24-hour excursions to 0% DO were not. Early and middle phase excursions had a greater impact on VCD, viability, and titer. Middle and late phase excursions tended to have a larger effect on product quality. The DOE models disclosed herein were well suited for modeling final viability, titer, purity, charge variant profile, and fucosylated glycans. Exemplary prediction models are shown in FIGS. 13-18.

An exemplary correlation between a predicted final viability and actual final viability is shown in FIG. 13A, with an R² of 0.7772. A 3D surface for a final viability model for a middle phase excursion is shown in FIG. 13B.

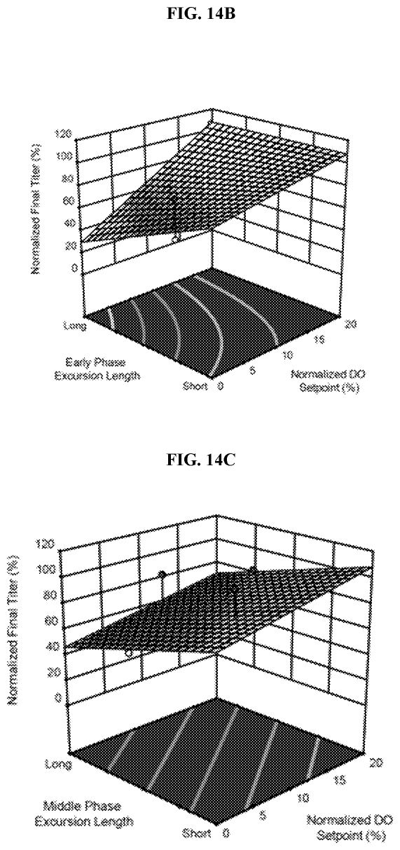

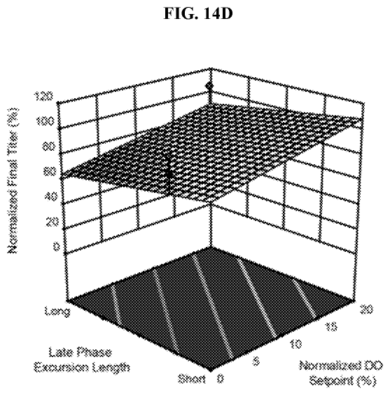

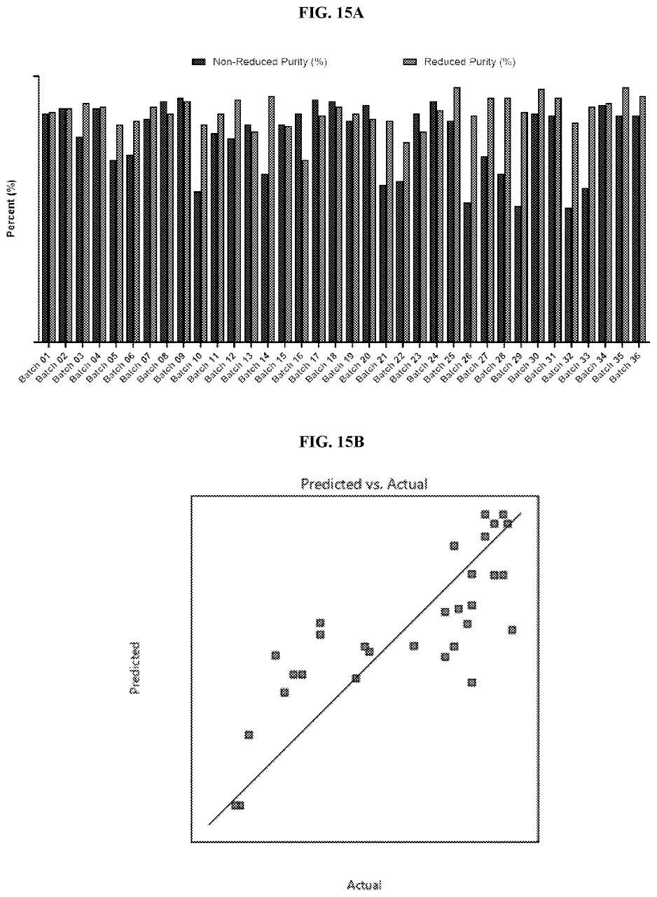

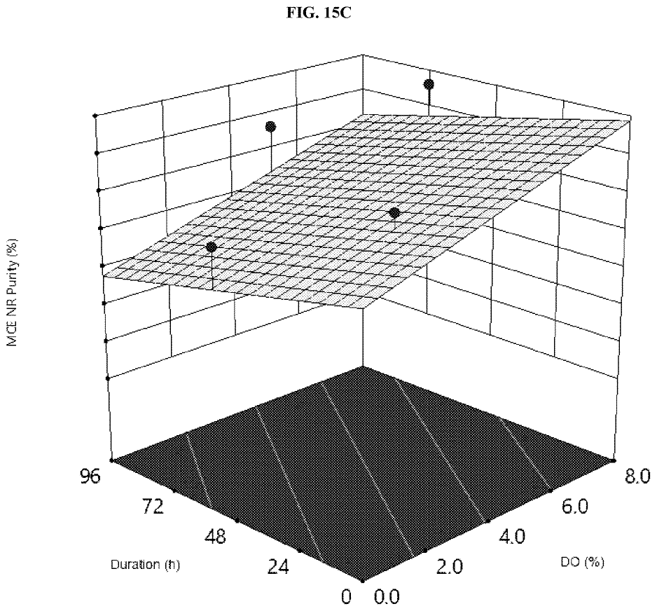

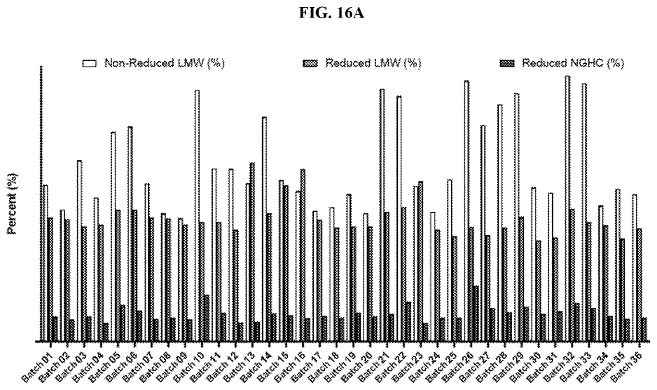

An exemplary correlation between a predicted final titer and actual final titer is shown in FIG. 14A, with an R² of 0.7283. A 3D surface for a normalized final titer model is shown in FIG. 14B (early phase), FIG. 14C (middle phase), and FIG. 14D (late phase).

FIG. 15A shows the results of an analysis of protein purity across each of the thirty-six mAb1 batches using R MCE and NR MCE. FIG. 15B shows an exemplary correlation between predicted purity as measured by NR MCE and actual purity as measured by NR MCE, with an R² of 0.5963. A 3D surface for a NR MCE purity model for a middle phase excursion is shown in FIG. 15C.

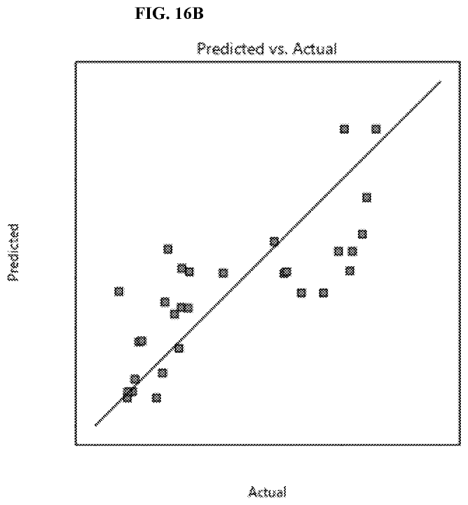

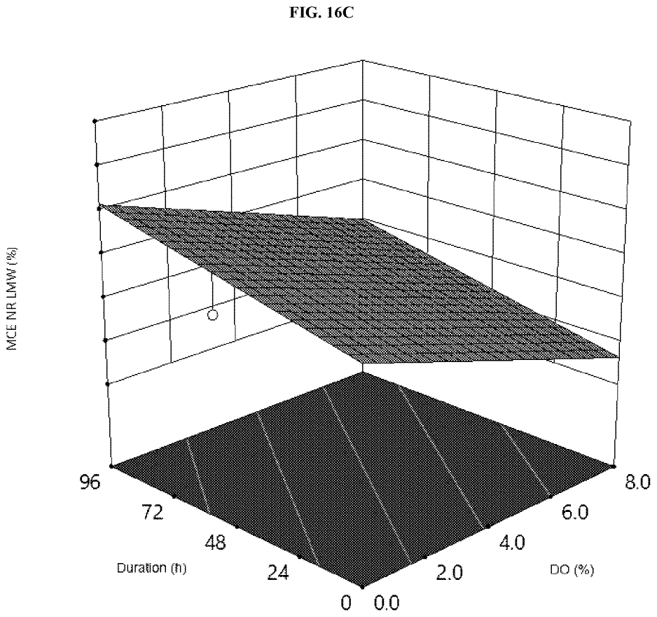

FIG. 16A shows the results of an analysis of LMW species and NGHC across each of the thirty-six mAb1 batches using R MCE and NR MCE. FIG. 16B shows an exemplary correlation between predicted LMW species as measured by NR MCE and actual LMW species as measured by NR MCE, with an R² of 0.5515. A 3D surface for a NR MCE LMW model for a middle phase excursion is shown in FIG. 16C.

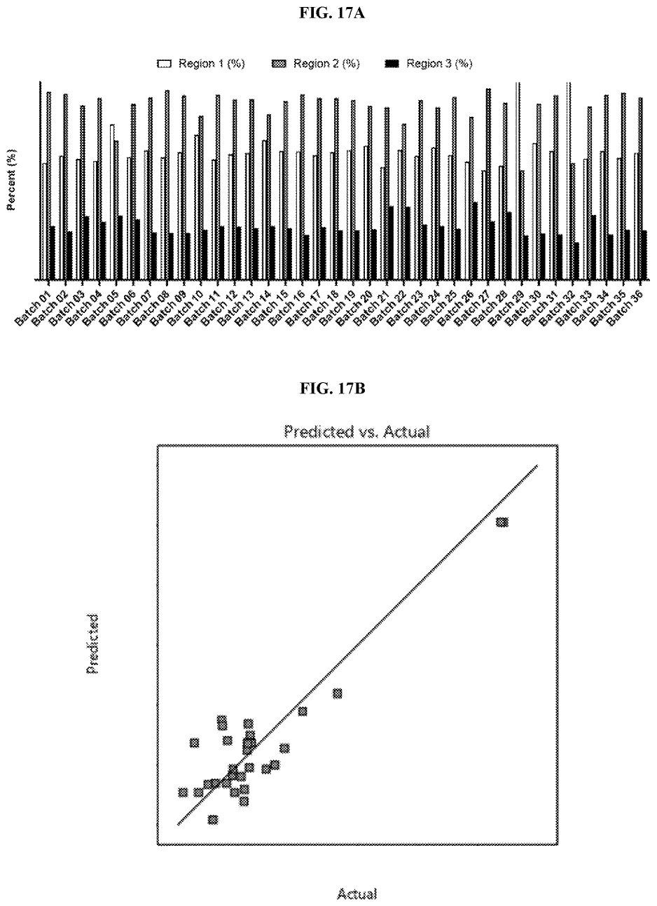

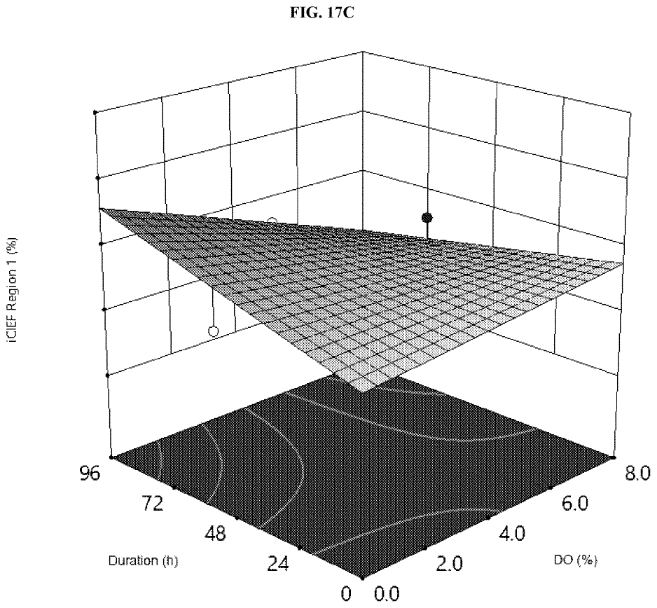

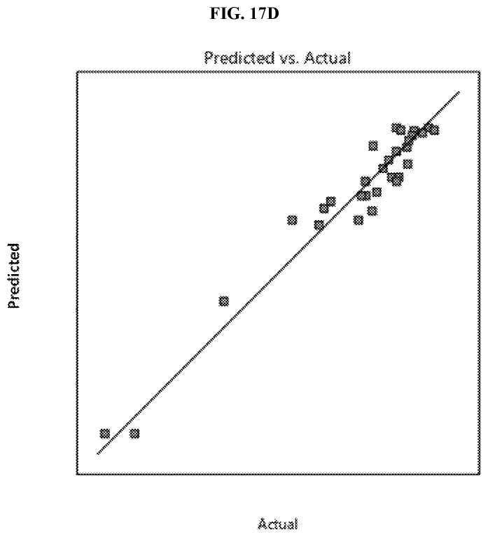

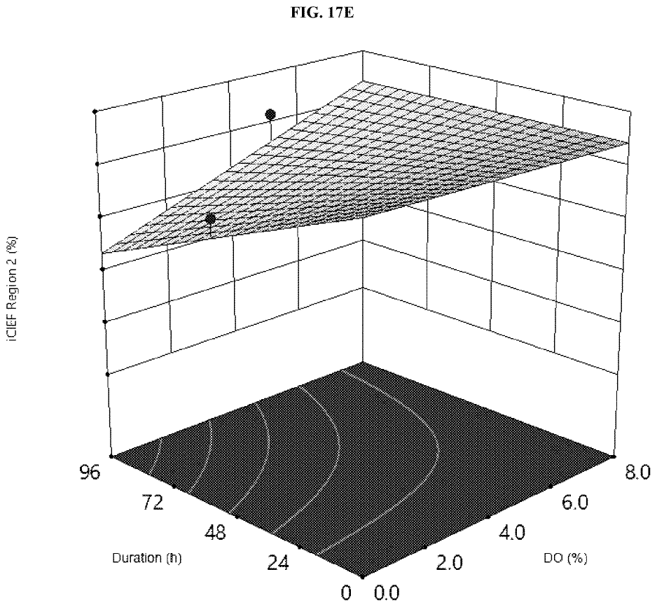

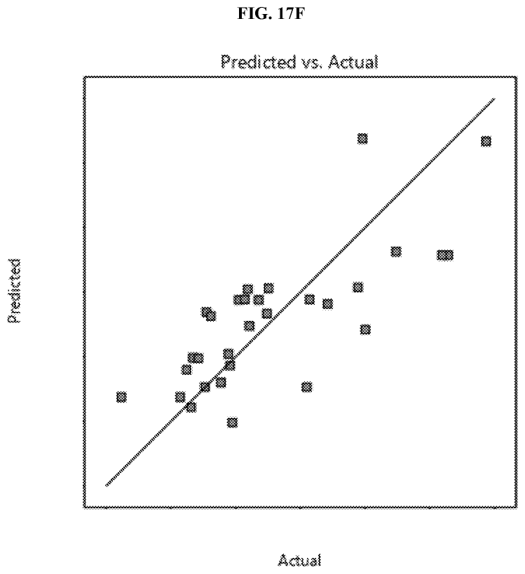

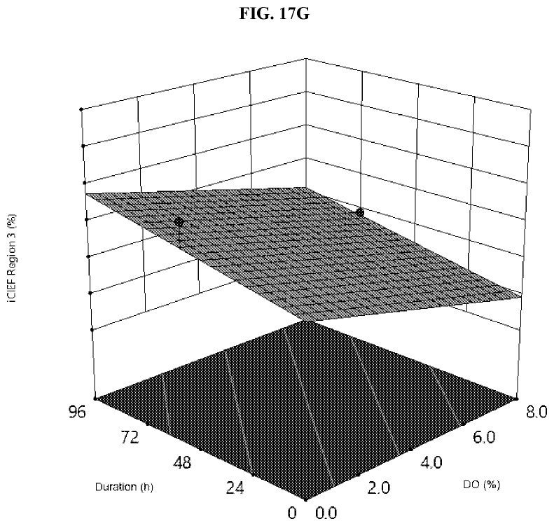

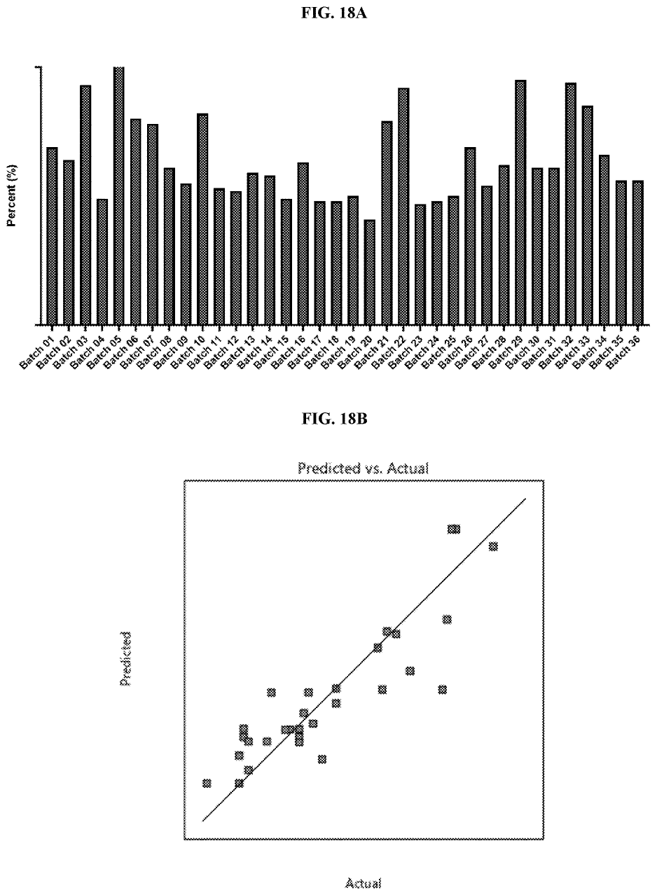

FIG. 17A shows the results of an analysis of charge variants across each of the thirty-six mAb1 batches using iCIEF. FIG. 17B shows an exemplary correlation between predicted iCIEF Region 1(%) and actual iCIEF Region 1(%), with an R² of 0.8311. A 3D surface for an iCIEF Region 1(%) model for a middle phase excursion is shown in FIG. 17C. FIG. 17D shows an exemplary correlation between predicted iCIEF Region 2(%) and actual iCIEF Region 2(%), with an R² of 0.9397. A 3D surface for an iCIEF Region 2(%) model for a middle phase excursion is shown in FIG. 17E. FIG. 17F shows an exemplary correlation between predicted iCIEF Region 3(%) and actual iCIEF Region 3(%), with an R² of 0.5820. A 3D surface for an iCIEF Region 3(%) model for a middle phase excursion is shown in FIG. 17G.

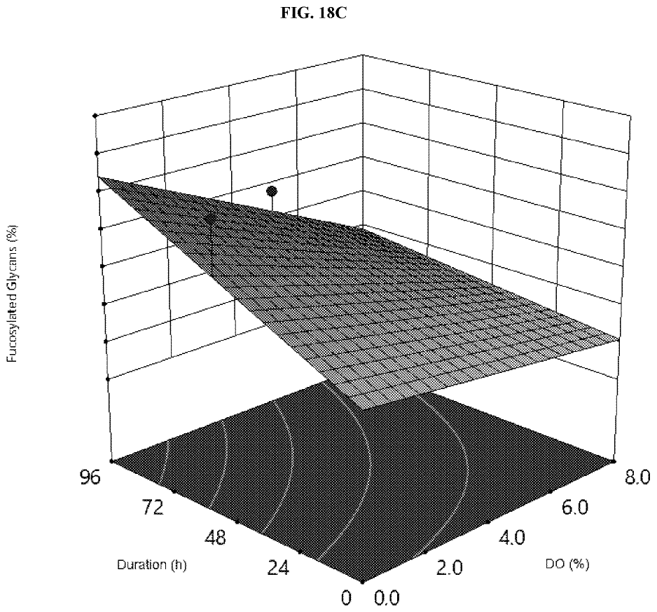

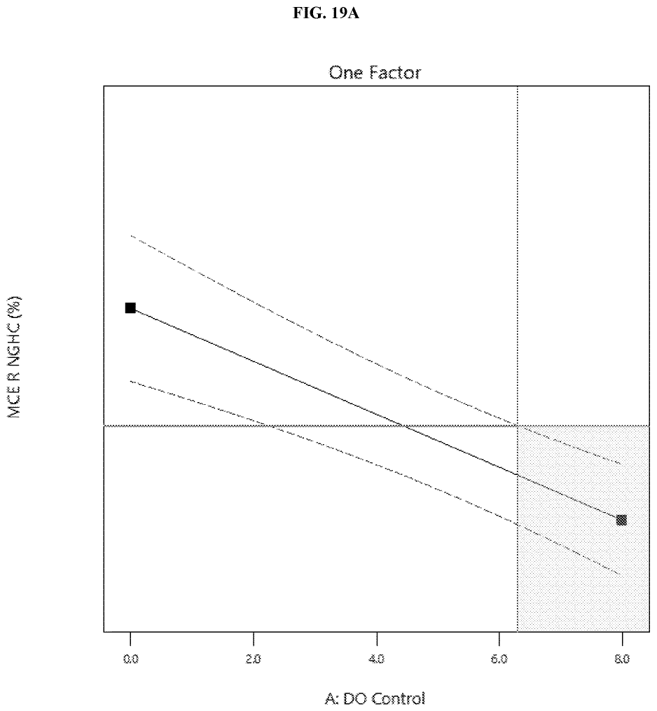

FIG. 18A shows the results of an analysis of fucosylated glycans across each of the thirty-six mAb1 batches. FIG. 18B shows an exemplary correlation between predicted fucosylated glycans and actual fucosylated glycans, with an R² of 0.7441. A 3D surface for a fucosylated glycan model for a middle phase excursion is shown in FIG. 18C.

A summary of the results of the mAb1 DOE study is shown in Table 7 below.

TABLE 7
Summary of mAb1 DOE study

| | Characterized | |
|---|---|---|
| Input | Range | |
| Parameter | (Control) | Results |
| DO Setpoint | 0.0-8.0 | Statistically significant in determining a direct main effect for |
| (%) | (40.4) | final viability, final titer, purity by NR MCE, iCIEF Region 2, |
| | | other glycans, G0, G1F, G1(6), G1F(3), G2, and G2F. |
| | | Statistically significant in determining an inverse main effect |
| | | for purity by R MCE, LMW by NR MCE, iCIEF Region 1, |
| | | iCIEF Region 3, fucosylated glycans, G0F, and mannose-5. |
| | | Constituent to a statistically significant interaction in |
| | | determining final viability, purity by R MCE, NGHC by R |
| | | MCE, iCIEF Region 1, iCIEF Region 2, iCIEF Region 3, |
| | | fucosylated glycans, other glycans, G0, G0F, G1F, mannose-5, |
| | | G1(6), G1F(3), G2, and G2F. |
| Duration of | 24-96 | Statistically significant in determining a direct main effect for |
| DO Excursion | (0) | final viability, NGHC by R MCE, iCIEF Region 2, iCIEF |
| in Early Phase | | Region 3, G0, and G0F. |
| (hours) | | Statistically significant in determining an inverse main effect |
| | | for final titer, iCIEF Region 1, G1F, G1(6), G1F(3), G2, and |
| | | G2F. |
| | | Constituent to a statistically significant interaction in |
| | | determining final viability, final titer, NGHC by R MCE, iCIEF |
| | | Region 1, iCIEF Region 2, iCIEF Region 3, G0, G0F, G1F, |
| | | G1(6), G1F(3), G2, and G2F. |
| Duration of | 24-96 | Statistically significant in determining a direct main effect for |
| DO Excursion | (0) | LMW by NR MCE, iCIEF Region 1, fucosylated glycans, G1F, |
| in Middle | | and G1F(3). |
| Phase (hours) | | Statistically significant in determining an inverse main effect |
| | | for final viability, final titer, purity by R MCE, iCIEF Region 2, |
| | | other glycans, and mannose-5. |
| | | Constituent to a statistically significant interaction in |
| | | determining final viability, purity by R MCE, iCIEF Region 1, |
| | | iCIEF Region 2, fucosylated glycans, other glycans, G0, G1F, |
| | | G1(6), and G1F(3). |
| Duration of | 24-96 | Statistically significant in determining a direct main effect for |
| DO Excursion | (0) | final titer, purity by R MCE, LMW by NR MCE, fucosylated |
| in Late Phase | | glycans, and mannose-5. |
| (hours) | | Statistically significant in determining an inverse main effect |
| | | for final viability, other glycans, G0, andG1(6). |
| | | Constituent to a statistically significant interaction in |
| | | determining final viability, final titer, purity by R MCE, G0, |
| | | and mannose-5. |

Prediction profiles were used to determine DO excursion ranges that resulted in acceptable product quality (within a 95% confidence interval), for excursions in each cell culture phase. For example, a longest acceptable excursion was assessed by determining the longest duration at the highest evaluated DO that resulted in acceptable product quality. Alternately, a largest acceptable excursion magnitude was assessed by determining the lowest DO at the shortest evaluated excursion time that resulted in acceptable product quality. Early phase excursions at 8.0% were acceptable for the entire evaluated excursion duration range, so alternatively the lowest tolerable DO for a 96-hour excursion in the early phase was assessed. “Acceptable product quality” as used herein refers to a product that meets all performance targets for an established manufacturing process; acceptability may be determined by other similar or analogous standards, for example, in-process specifications or specification limits.

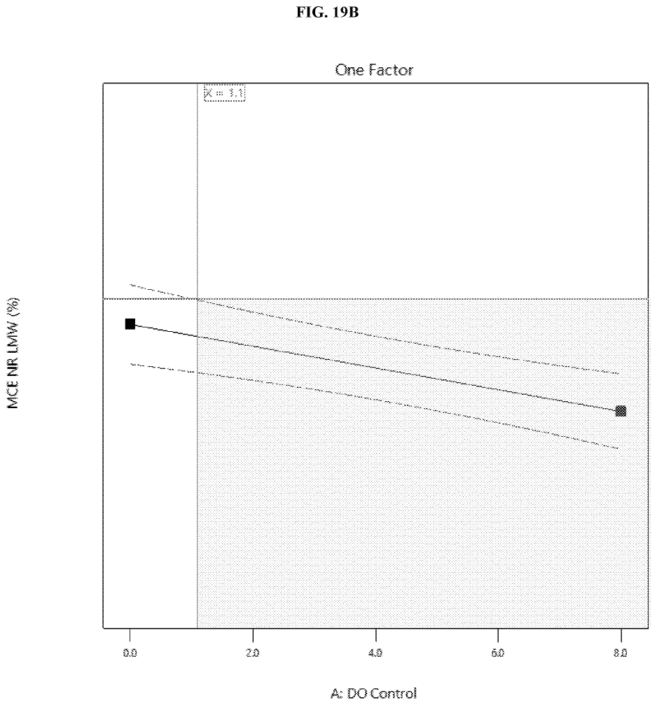

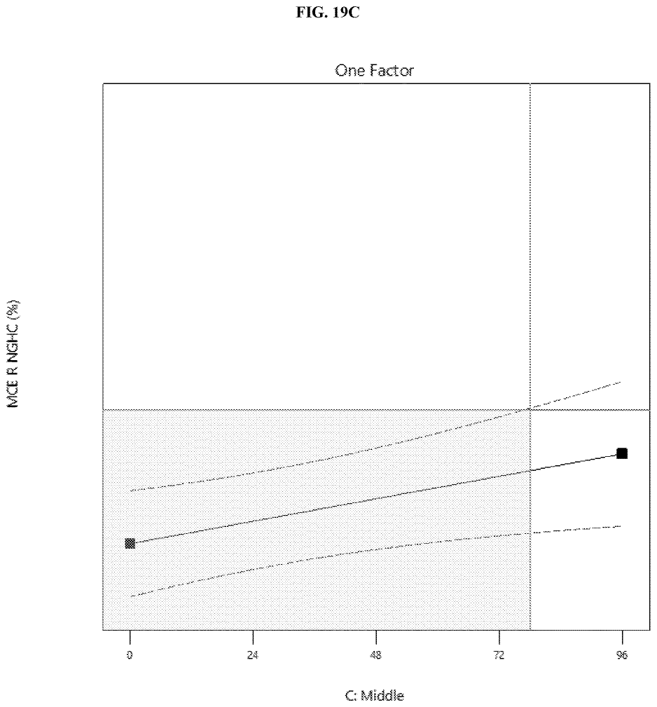

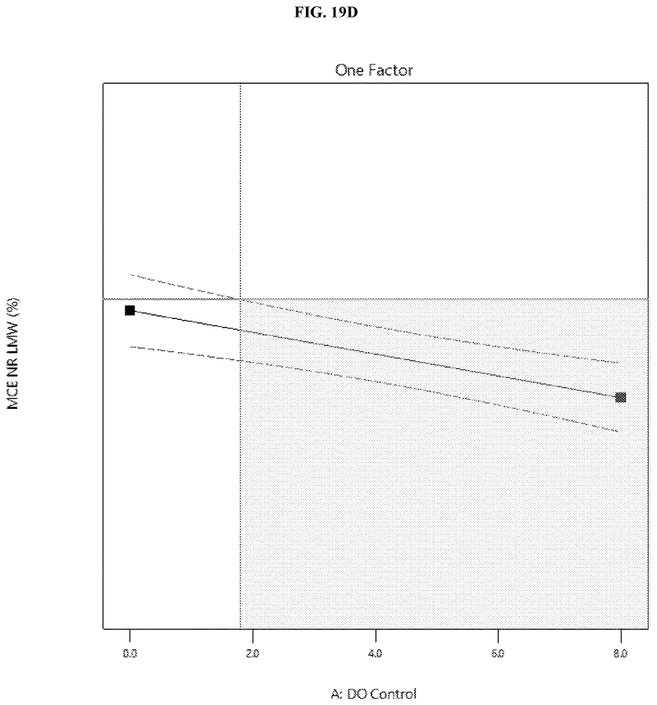

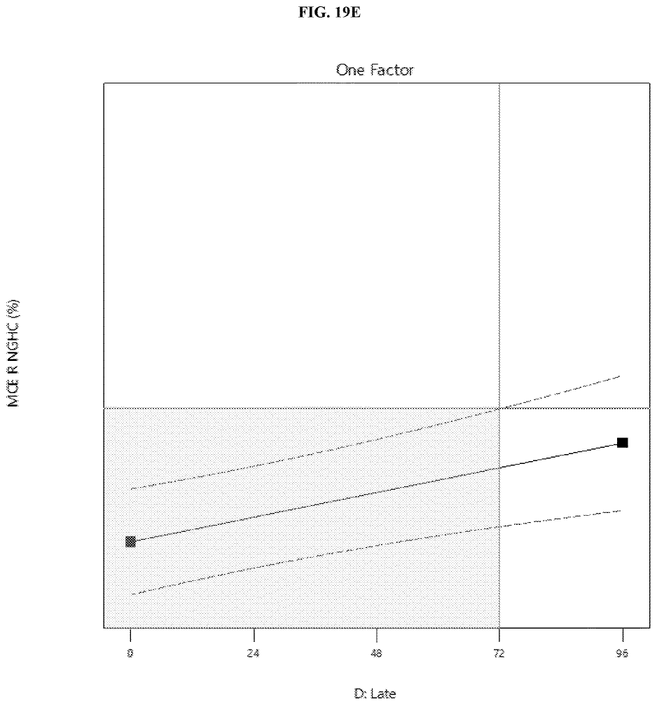

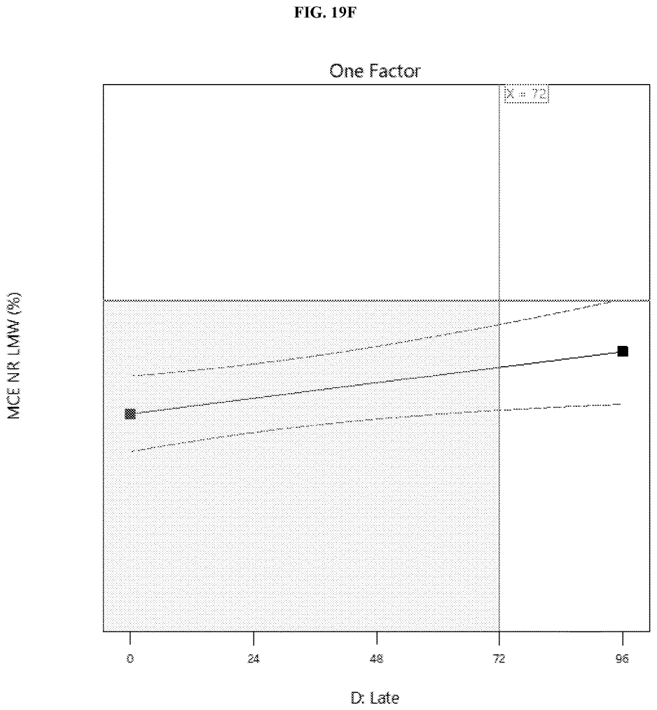

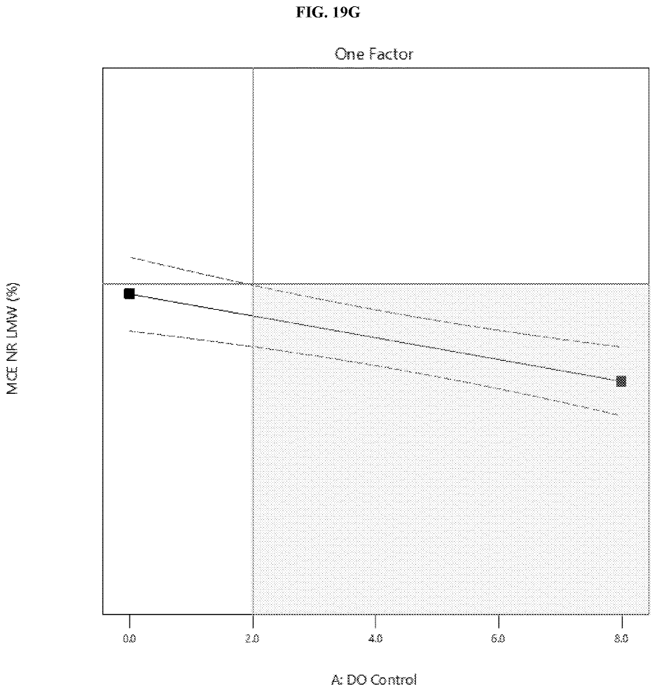

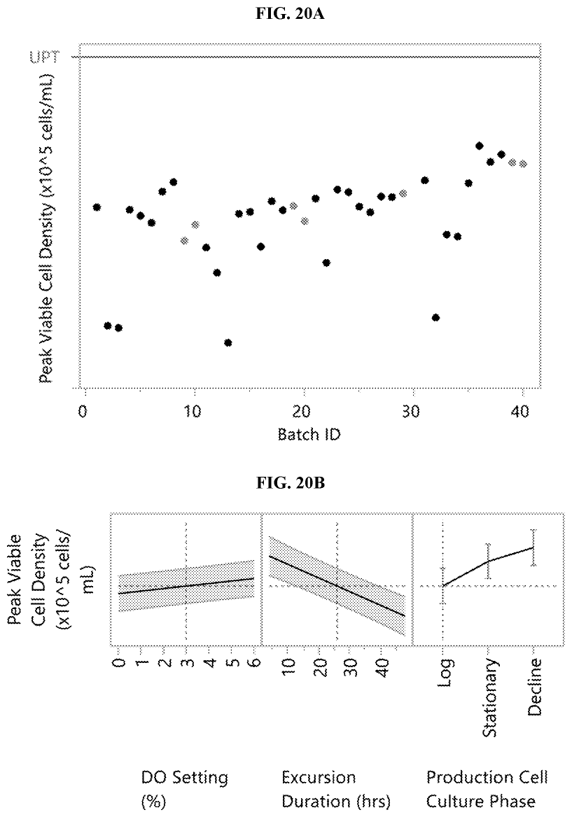

FIG. 19 shows exemplary predictions for the lowest tolerable DO level during a fixed length excursion in each phase, or the longest tolerable excursion with a fixed DO level in each phase. Only limiting responses for the particular protein studied are shown, but it should be understood that alternative proteins may have alternative product quality attributes that serve as a limiting factor. A corresponding summary of acceptable DO excursion properties based on product quality is set forth in Table 8. This provides an example of how DO excursions observed in a manufacturing process can be evaluated in order to predict which parameters fall within acceptable deviation, which should result in termination of a batch, and the particular product qualities affected.

TABLE 8
Acceptable DO excursion ranges based on product quality

| | DO | | |
|---|---|---|---|
| | Excursion | | |
| Input | Set | Excursion | |
| Parameters | Point | Length | Limiting Response(s) |
| Early Phase | 6.3% | 96 hours | NGHC R MCE |
| | 1.1% | 24 hours | LMW NR MCE |
| Middle Phase | 8.0% | 78 hours | NGHC R MCE |
| | 1.8% | 24 hours | LMW NR MCE |
| Late Phase | 8.0% | 72 hours | LMW NR MCE, |
| | | | NGHC R MCE |
| | 2.0% | 24 hours | LMW NR MCE |

These findings highlight the relationship between DO control and product quality, demonstrating process robustness and offering leverage to reduce the frequency of excursion investigations through the quality system. The methods disclosed herein for using models to relate DO excursion characteristics to cell culture outcomes and product quality can be used to determine whether a cell culture that has undergone a DO excursion should undergo a modified manufacturing process, should be escalated in the quality system, or should be terminated.

For example, if a DO excursion is detected in a cell culture batch, the effect of the DO excursion on predicted product quality can be determined using a model correlating DO excursion properties to product quality. If the predicted product quality falls outside of a performance target or specification limit, it can be determined that the batch will fail to meet requirements, and the batch can be terminated. Alternatively, if the predicted product quality is still within specification limits, other process changes can be made to accommodate the tolerable DO excursion, for example: extending the duration of a batch in order to compensate for reduced cell growth and reduced productivity; increasing the sparging rate to improve dissolved oxygen levels; modifying the agitation of the bioreactor for distribution of dissolved gas; or modifying a feeding schedule in order to accommodate a reduced cell growth rate.

This method of proactively terminating a batch provides a significant advantage in time and resources compared to conventional methods. For example, in the case of a cell culture producing a recombinant protein of interest, conventional methods require growing a failed batch to the harvest day, harvesting and purifying the protein of interest, and subjecting the protein of interest to quality testing before determining that the protein of interest has failed to meet specification limits and must be discarded. Therefore, the methods of the present invention allow for substantial gains in time and resources, and ultimately an increased efficiency and throughput for recombinant protein production, compared to conventional methods.

While conventional PAR studies may be used to indicate an acceptable range for DO excursions, they are limited to one DO setpoint for the entire duration of the production bioreactor unit operation. Therefore, a PAR study may determine the acceptable range of DO setpoint for the entirety of the unit operation, but may not reliably determine the acceptable range of a transient DO excursion. The results of the mAb1 DO excursion study described in this Example compared to a conventional PAR study of mAb1 DO are set forth in Table 9 below.

TABLE 9
Proven acceptable range versus transient excursion study results

| Study Type | Study Description | Results |
|---|---|---|
| mAb1 production | Studied static DO setpoint for the entire duration of the | PAR: 14.3-70.4% |
| PAR study | production bioreactor unit operation. Evaluated 0.0-90.9% | Robustness: |
| | | 20.0-62.0% |
| mAb1 DO | Studied variable length transient drops in DO across | For an |
| excursion | different cell growth phases of the production bioreactor | excursion in |
| study in the | unit operation. Evaluated 0.0-8.0% drops for 24-96 | any phase, |
| production | hours across all 3 cell growth phases | the DO can |
| bioreactor | | drop down |
| | | to 4.0% for up |
| | | to 12 hours |

As shown from the comparison of study results, the transient excursion study allows for a substantially extended acceptable range for transient DO excursions compared to what would be determined based on a PAR study, from 14.3% to 4.0% for mAb1. Using this extended acceptable range from an excursion study, cell culture batches that would be considered unacceptable if using a more restricted acceptable range can instead be allowed to proceed to harvest, further resulting in increased efficiency and throughput compared to conventional methods.

### Example 2. Dissolved Oxygen Excursion Design of Experiments for Dupilumab

### 2.1. Study Parameters

A laboratory-scale design of experiments study was performed to determine any impact of transient dissolved oxygen excursions during the production bioreactor unit operation of dupilumab, a monoclonal IgG4 antibody. The input parameters selected are the same as those set forth in Table 1, and the output parameters selected include those set forth in Table 2, although the specific values tested for each parameter differed compared to the mAb1 study, as detailed below. Additional output parameters were assessed, including peak viable cell density (VCD) and Chinese Hamster Ovary (CHO) host cell protein (HCP) content. The early phase corresponded to production day 1 through day 4, the middle phase corresponded to production day 5 through day 7, and the late phase corresponded to production day 8 through the final day of production.

The experimental design of the study was created by applying design of experiment techniques using the statistical software Design-Expert. Table 10 summarizes the settings selected for each input parameter included in this study.

TABLE 10
Study parameters

| | Nominal | Lowest | Highest |
|---|---|---|---|
| Parameter | Conditions | Setting | Setting |
| Duration of DO | 0 | 4 | 48 |
| excursion (hours) | | | |
| Phase of DO | No excursions. | Log (starting day 1) | |
| excursion | | Stationary (starting day 5) | |
| (process day) | | Decline (starting day 8) | |
| DO setpoint during | 25.0 | 0.0 | 6.0 |
| excursion (%) | | | |

Forty batches of cells were produced for this study. Thirty-two batches were designated as factorial points to test different combinations of high and low factorial settings. Eight batches were designated as center points with no planned DO deviations. The forty batches were split into four separate groups, termed blocks, of ten batches per block. Batches were randomly assigned to each block, apart from ensuring two center points in each block. One cell bank vial was thawed and expanded for each block.

The block assignments and study parameter settings for each batch are summarized in Table 11.

TABLE 11
Study design

| | | DO | Excursion | |
|---|---|---|---|---|
| Run | | Setting | Duration | Excursion |
| ID | Block | (%) | (hours) | Phase |
| 1 | 1 | 0.0 | 4 | Log |
| 2 | | 2.2 | 48 | Log |
| 3 | | 2.2 | 48 | Log |
| 4 | | 0.0 | 4 | Stationary |
| 5 | | 0.0 | 4 | Stationary |
| 6 | | 5.4 | 48 | Stationary |
| 7 | | 6.0 | 48 | Stationary |
| 8 | | 6.0 | 4 | Decline |
| 9 | | 25.0 | 0 | N/A |
| 10 | | 25.0 | 0 | N/A |
| 11 | 2 | 6.0 | 21 | Log |
| 12 | | 0.0 | 25 | Log |
| 13 | | 0.0 | 25 | Log |
| 14 | | 6.0 | 4 | Stationary |
| 15 | | 4.0 | 13 | Stationary |
| 16 | | 3.0 | 26 | Stationary |
| 17 | | 0.0 | 4 | Decline |
| 18 | | 2.2 | 48 | Decline |
| 19 | | 25.0 | 0 | N/A |
| 20 | | 25.0 | 0 | N/A |
| 21 | 3 | 3.0 | 4 | Log |
| 22 | | 0.0 | 48 | Stationary |
| 23 | | 6.0 | 13 | Decline |
| 24 | | 3.0 | 26 | Decline |
| 25 | | 0.0 | 26 | Decline |
| 26 | | 3.0 | 26 | Decline |
| 27 | | 1.0 | 48 | Decline |
| 28 | | 6.0 | 48 | Decline |
| 29 | | 25.0 | 0 | N/A |
| 30 | | 25.0 | 0 | N/A |
| 31 | 4 | 5.4 | 4 | Log |
| 32 | | 3.0 | 48 | Log |
| 33 | | 6.0 | 48 | Log |
| 34 | | 0.0 | 26 | Stationary |
| 35 | | 6.0 | 26 | Stationary |
| 36 | | 1.0 | 48 | Stationary |
| 37 | | 3.0 | 4 | Decline |
| 38 | | 4.0 | 25 | Decline |
| 39 | | 25.0 | 0 | N/A |
| 40 | | 25.0 | 0 | N/A |

The sample collection and testing strategy is summarized in Table 12. Sample volumes were approximate. Multiple tests could be run from the same sample.

TABLE 12
Sampling and testing strategy

| | Sample | |
|---|---|---|
| Time of Sampling | Volume | Test |
| Production batch days 0 | 2 mL | NOVA BioProfile ® Flex Analyzer (viable cell density/ |
| through harvest | | cell culture viability/cell culture characteristics) |
| Production batch day 0 | 2 mL | Sampling for biological state assessment |
| Production batch days 1 | 1 mL | Sampling for biological state assessment |
| through harvest | | |
| Production batch DO | 1 mL | Sampling for biological state assessment |
| excursion end point | | |
| Production batch days | 5 mL | Protein titer determination by process sciences HPLC |
| 4, 6, 8-11 | | |
| Production batch day | 5 mL | Protein titer determination by HPLC for early phase |
| of harvest | | clinical monoclonal antibodies |
| Post-affinity | 10 mL | N-glycan analysis by RapiFluor-MS labeling with |
| chromatography | | hydrophilic interaction chromatography |
| (Protein A-viral | | Antibody charge variant analysis by imaging capillary |
| inactivated pool) | | isoelectric focusing |
| | | Purity analysis of in process test articles by PICO MCE |
| | | Monoclonal antibody purity by size exclusion ultra-high |
| | | performance liquid chromatography |

### 2.2. Laboratory-Scale Design of Experiments Screening

Seed expansion in the laboratory-scale system followed an expansion schedule representative of the manufacturing process. Culture volumes during shaker flask and cellbag process steps are identical between manufacturing and laboratory-scale systems. Volumes during laboratory-scale seed expansion and production stainless steel bioreactor (SSB) unit operations (N-3, N-2, N-1, and production bioreactors) were scaled to match ratios of cell culture to fresh medium used during the manufacturing process. Volumes of any nutrient additions within the laboratory-scale system were also scaled appropriately to match the ratio employed within the manufacturing-scale system.

Recombinant CHO cells expressing a recombinant IgG4 antibody, dupilumab, were thawed and inoculated into a 500 mL shaker flask with CHO medium. After sufficient time for cell growth, cells were sequentially transferred to a 2 L cellbag, a 20 L cellbag, a 2 L N-3 bioreactor, a 2 L N-2 bioreactor, a 2 L N-1 bioreactor, and finally a 250 mL production bioreactor. Each cell culture vessel could be used to inoculate multiple subsequent vessels within a single block of experiments. Each 2 L bioreactor step could reuse the 2 L bioreactor of the previous step, by transferring a portion of the cell culture out of the bioreactor and into a media bag containing fresh media, aseptically draining and discarding the remaining cell culture from the bioreactor, and returning the contents of the media bag to the empty 2 L bioreactor. Each production bioreactor was sampled as specified in Table 12. DO setpoints were adjusted as specified in Table 11, and then returned to the default setpoint of 25.0%. Bioreactors were sampled and analyzed as specified in Table 12 until the batch duration of about 240-264 hours (10-11 days) had elapsed, and then proceeded to harvest.

Production bioreactor material was harvested following completion of the production unit operation. Harvest set-up and pre-use filter flushes were performed in a manner representative of the manufacturing process. Volumes of any water and buffer flushes within the laboratory-scale system were scaled appropriately to match or exceed the buffer volume to membrane surface area ratio employed within the manufacturing-scale system. Briefly, cell culture samples were subjected to filtration, Protein A affinity chromatography, and viral inactivation prior to subsequent testing.

Cell pellets and spent media can be further processed for assessing gene expression levels, protein expression analysis, and metabolomics. Differences in gene expression, protein expression, and metabolite levels can be compared between production bioreactors with varying excursions to determine how DO excursions impact cellular health and metabolism. Unique molecular profiles found to be associated with particular excursion features likely precede phenotypic cellular changes, and could serve as biomarkers for understanding and predicting cell culture outcomes.

### 2.3. Impact of DO Excursions on Product Quality and Model Analysis

The cell culture batches as described in Table 11 and the dupilumab products thereof were subjected to quality testing to determine an effect of dissolved oxygen excursions, and to develop models for predicting product quality based on dissolved oxygen excursions. None of the batches studied fell outside of process performance limits, except for one batch for iCIEF Region 3.

The peak viable cell density of each of the forty batches is shown in FIG. 20A, with control batches marked in gray. Performance targets were offset compared to the manufacturing scale. Significant models were found for the terms DO setting, excursion duration, log phase excursions, decline phase excursions, excursion duration*log phase excursions, excursion duration*stationary phase excursions, and excursion duration*decline phase excursions. Prediction profiles relating peak viable cell density to representative factors are shown in FIG. 20B.

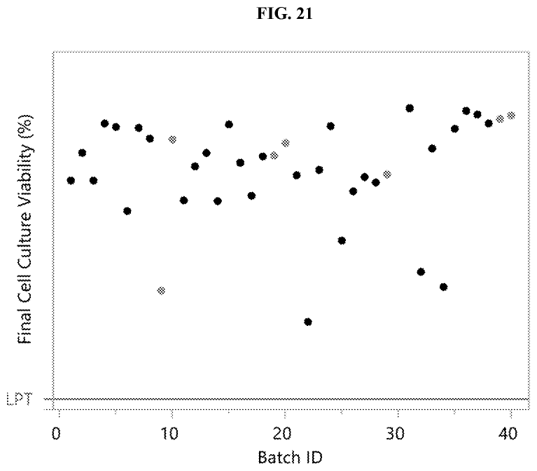

The final cell culture viability of each of the forty batches is shown in FIG. 21, with control batches marked in gray. Laboratory-scale viability was comparable to manufacturing-scale and no offset of performance targets was required. No significant model was found, so the mean model, or the mean of the overall data set, was the best predictor of process performance, and none of the evaluated excursion parameters in the evaluated ranges were significant in predicting viability.

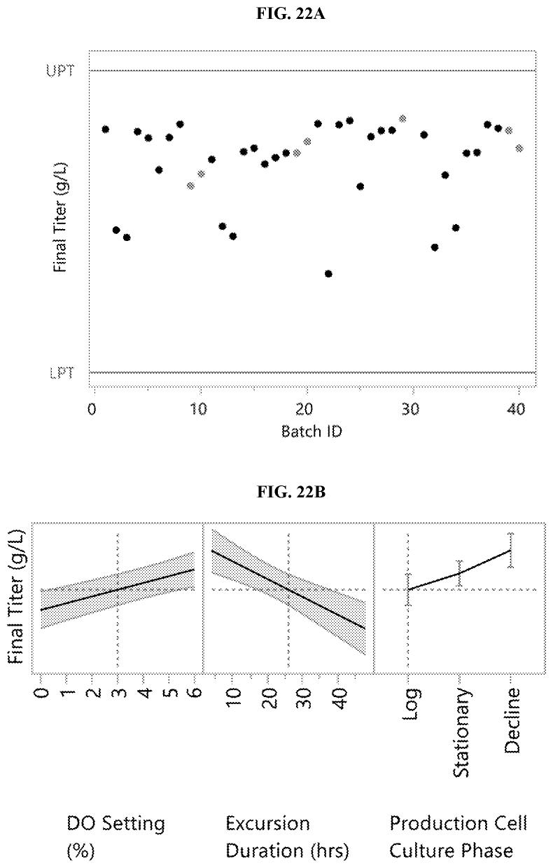

The final titer of each of the forty batches is shown in FIG. 22A, with control batches marked in gray. Performance targets were offset compared to the manufacturing scale. Significant models were found for the terms DO setting, excursion duration, log phase excursions, decline phase excursions, DO setting*excursion duration, excursion duration*log phase excursions, and excursion duration*decline phase excursions. Prediction profiles relating final titer to representative factors are shown in FIG. 22B.

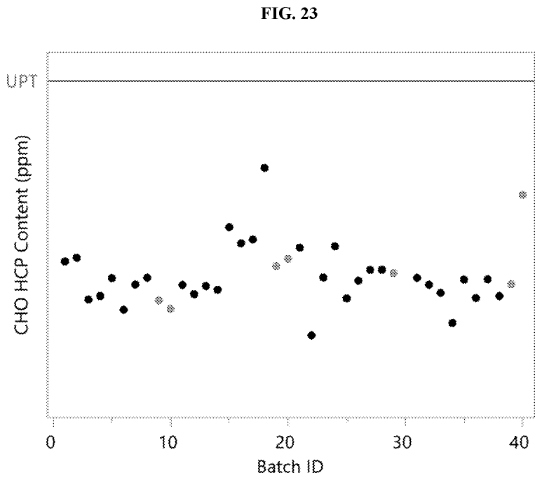

The Chinese hamster ovary (CHO) cell host cell protein (HCP) content of each of the forty batches is shown in FIG. 23, with control batches marked in gray. Laboratory-scale CHO HCP content was comparable to manufacturing-scale and no offset of performance targets was required. No significant model was found, so the mean model was the best predictor of process performance, and none of the evaluated excursion parameters in the evaluated ranges were significant in predicting CHO HCP content.

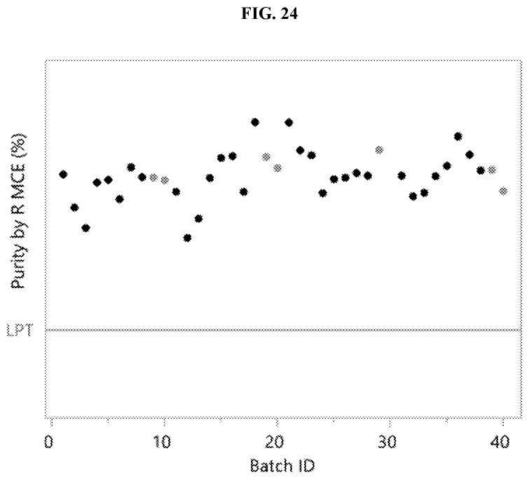

Purity by R MCE for each of the forty batches is shown in FIG. 24, with control batches marked in gray. Performance targets were offset compared to the manufacturing scale. No significant model was found, so the mean model was the best predictor of process performance.

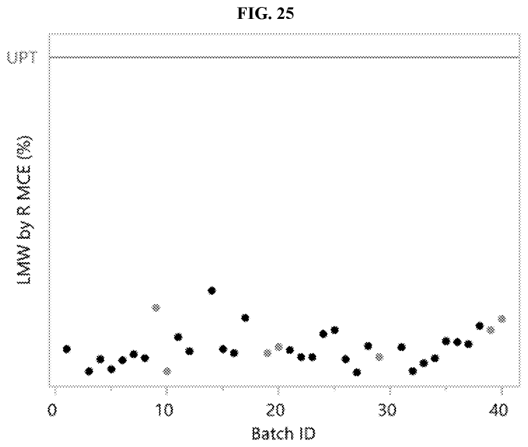

LMW species by R MCE for each of the forty batches is shown in FIG. 25, with control batches marked in gray. Performance targets were offset compared to the manufacturing scale. No significant model was found, so the mean model was the best predictor of process performance.

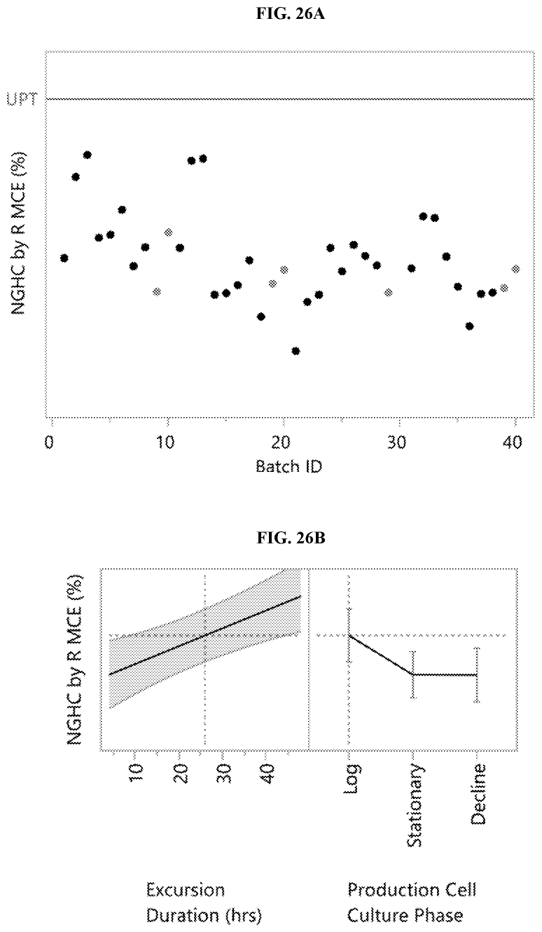

NGHC by R MCE for each of the forty batches is shown in FIG. 26A, with control batches marked in gray. Performance targets were offset compared to the manufacturing scale. Significant models were found for the terms log phase excursions and excursion duration*log phase excursions. Prediction profiles relating NGHC by R MCE to representative factors are shown in FIG. 26B.

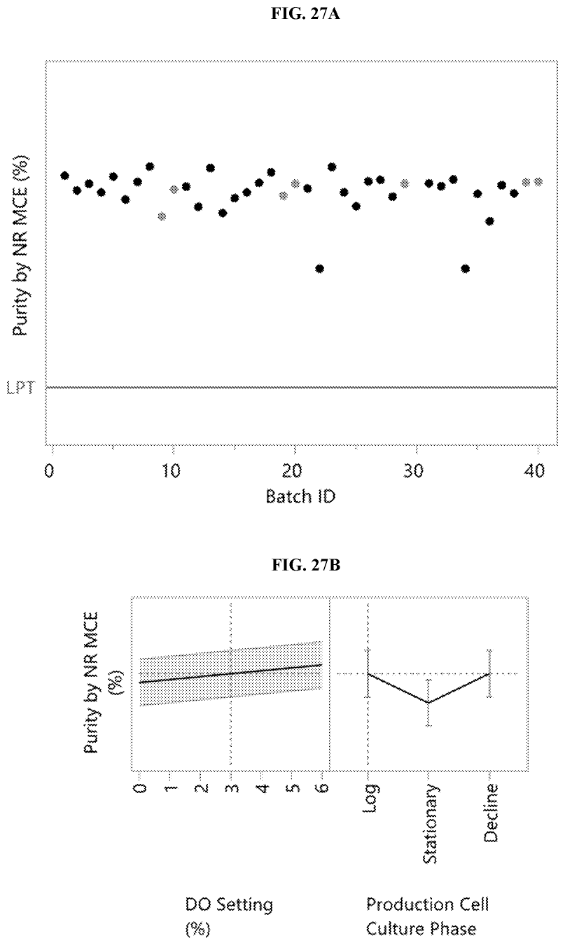

Purity by NR MCE for each of the forty batches is shown in FIG. 27A, with control batches marked in gray. Performance targets were offset compared to the manufacturing scale. Significant models were found for the terms DO setting, stationary phase excursions, and decline phase excursions. Prediction profiles relating purity by NR MCE to representative factors are shown in FIG. 27B.

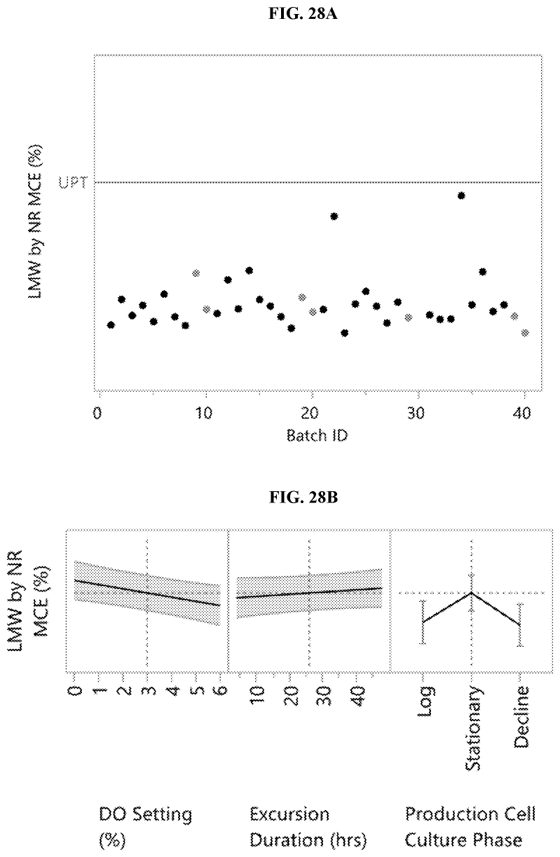

LMW by NR MCE for each of the forty batches is shown in FIG. 28A, with control batches marked in gray. Laboratory-scale LMW by NR MCE was comparable to manufacturing-scale and no offset of performance targets was required. Significant models were found for the terms DO setting, stationary phase excursions, and DO setting*excursion duration. Prediction profiles relating LMW by NR MCE to representative factors are shown in FIG. 28B.

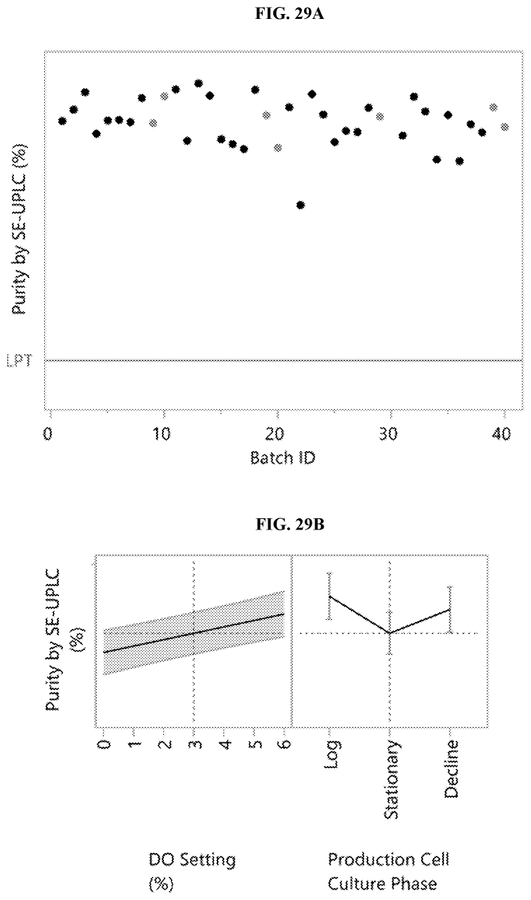

Purity by SE-UPLC for each of the forty batches is shown in FIG. 29A, with control batches marked in gray. Laboratory-scale purity by SE-UPLC was comparable to manufacturing-scale and no offset of performance targets was required. Significant models were found for the terms DO setting, log phase excursions, and stationary phase excursions. Prediction profiles relating purity by SE-UPLC to representative factors are shown in FIG. 29B.

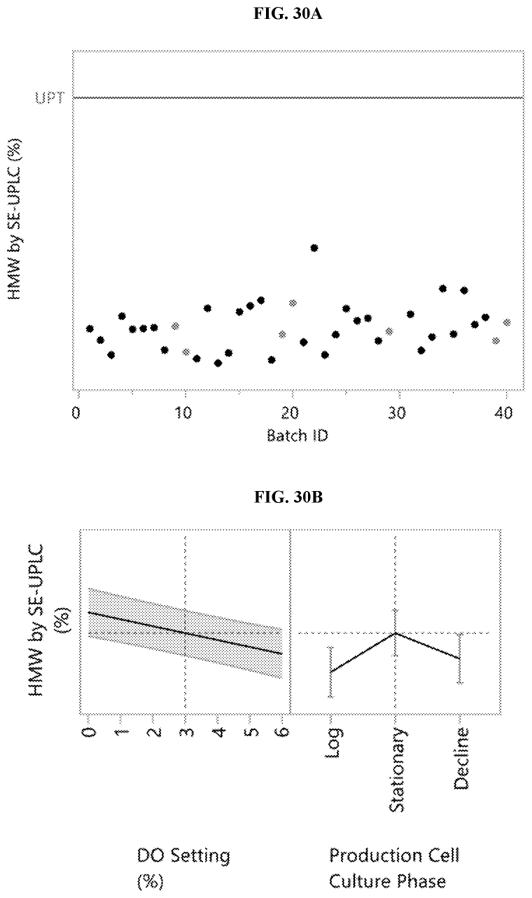

HMW by SE-UPLC for each of the forty batches is shown in FIG. 30A, with control batches marked in gray. Laboratory-scale HMW by SE-UPLC was comparable to manufacturing-scale and no offset of performance targets was required. Significant models were found for the terms DO setting, log phase excursions, and stationary phase excursions. Prediction profiles relating HMW by SE-UPLC to representative factors are shown in FIG. 30B.

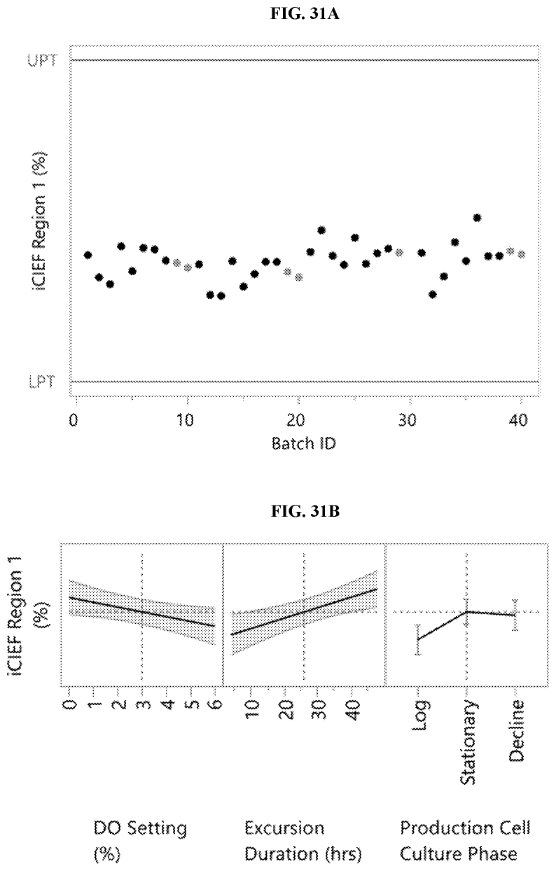

iCIEF Region 1 for each of the forty batches is shown in FIG. 31A, with control batches marked in gray. Performance targets were offset compared to the manufacturing scale. Significant models were found for the terms log phase excursions, stationary phase excursions, DO setting*log phase excursions, DO setting*stationary phase excursions, excursion duration*log phase excursions, and excursion duration*stationary phase excursions. Prediction profiles relating iCIEF Region 1 to representative factors are shown in FIG. 31B.

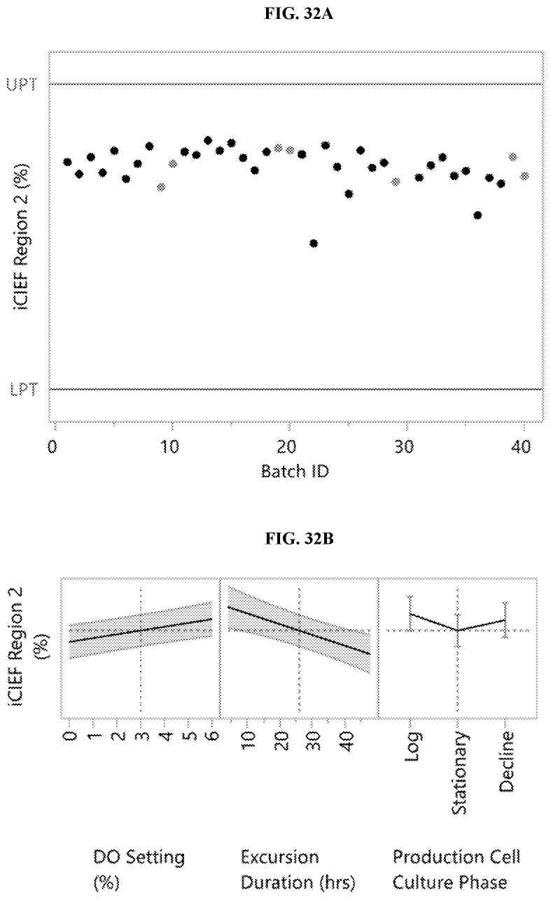

iCIEF Region 2 for each of the forty batches is shown in FIG. 32A, with control batches marked in gray. Performance targets were offset compared to the manufacturing scale. Significant models were found for the terms DO setting, excursion duration, stationary phase excursions, and excursion duration*stationary phase excursions. Prediction profiles relating iCIEF Region 2 to representative factors are shown in FIG. 32B.

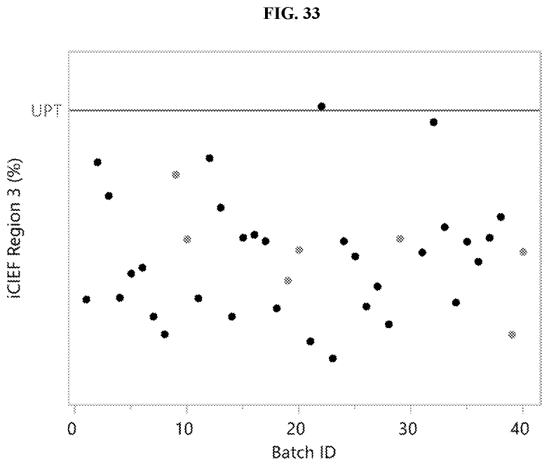

iCIEF Region 3 for each of the forty batches is shown in FIG. 33, with control batches marked in gray. Performance targets were offset compared to the manufacturing scale. No significant model was found, so the mean model was the best predictor of process performance.

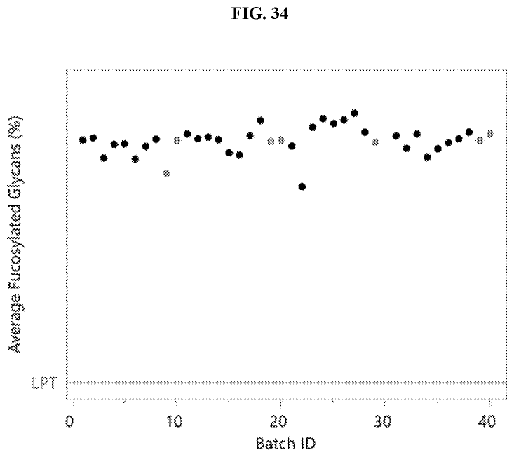

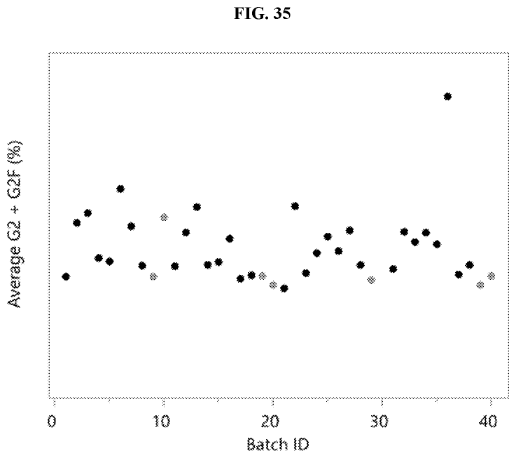

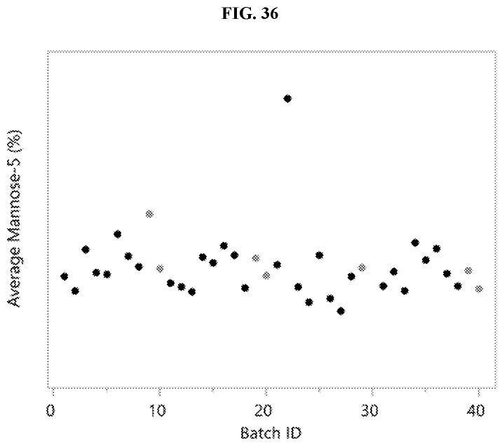

Average fucosylated glycans, average G2+G2F, and average mannose-5 for each of the forty batches is shown in FIG. 34, FIG. 35 and FIG. 36, respectively, with control batches marked in gray. No significant model was found for any of these glycosylation parameters.

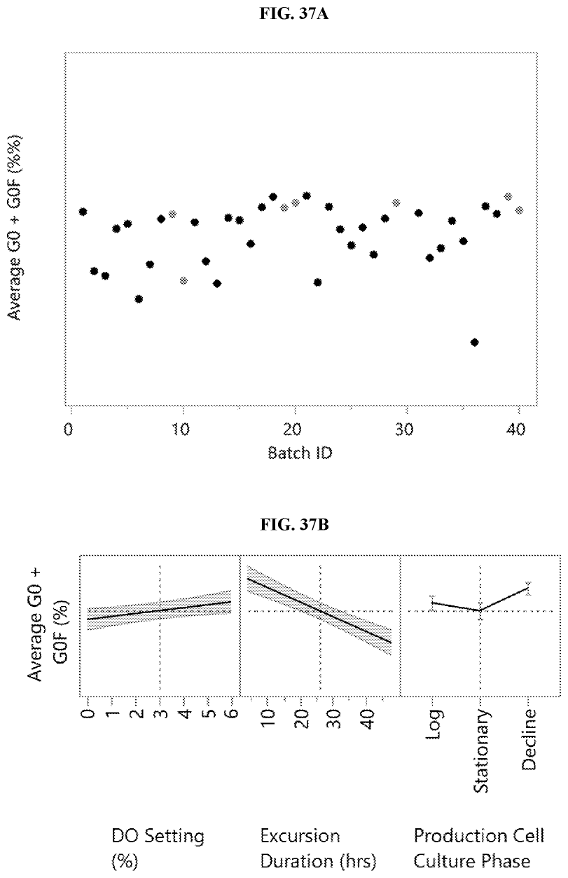

Average G0+G0F for each of the forty batches is shown in FIG. 37A, with control batches marked in gray. Significant models were found for the terms DO setting, excursion duration, stationary phase excursions, decline phase excursions, excursion duration*stationary phase excursions, and excursion duration*decline phase excursions. Prediction profiles relating G0+G0F to representative factors are shown in FIG. 37B.

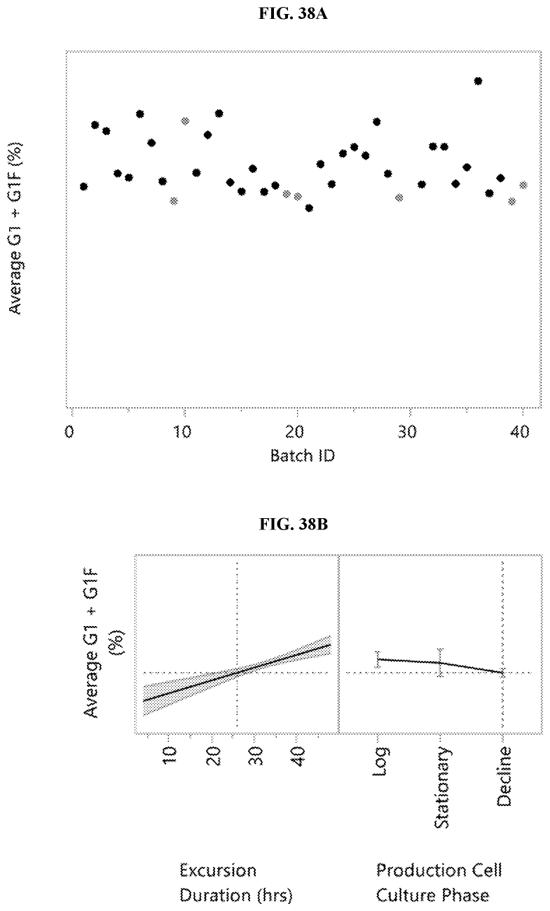

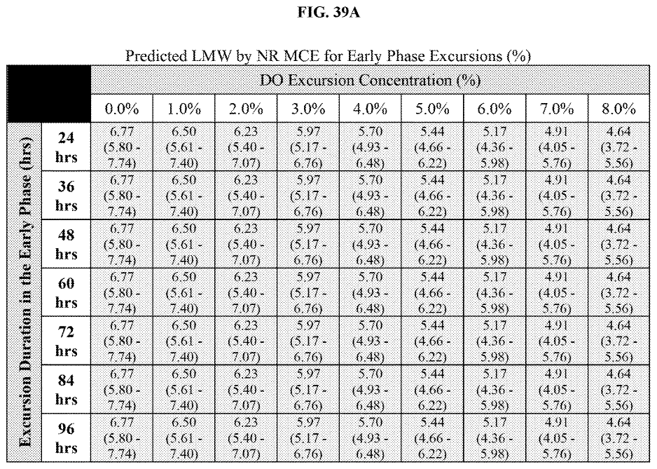

Average G1+G1F for each of the forty batches is shown in FIG. 38A, with control batches marked in gray. Significant models were found for the terms excursion duration and decline phase excursions. Prediction profiles relating G1+G1F to representative factors are shown in FIG. 38B.

This example, also in view of Example 1, demonstrates that the disclosed systems and methods for predicting cell culture health, process performance and product quality based on dissolved oxygen excursions can be applied generally across molecules, including for dupilumab. Instead of depending on product quality testing at the end of a lengthy and expensive production process, models can be used to predict product quality ahead of time in order to determine the course of a cell culture batch, including potentially modifying the course of a batch to compensate for a dissolved oxygen excursion, or terminating the batch if the final product is predicted to fail specifications. These systems and methods are a substantial improvement over the state of the art and can result in significant savings of time and expense in producing drug products.

### Example 3. Exemplary Case Studies

This Example sets forth case studies using the methods disclosed herein for controlling a cell culture batch by using a model to predict the effect of DO excursions on product quality.

### 3.1 Dynamic Dissolved Oxygen Excursion Models For Drug Product A

Models for predicting product quality attributes based on dissolved oxygen excursions for a first drug product, Drug Product A, are set forth below. Drug Product A has viral inactivation (VI) pool performance targets as outlined in Table 13.

TABLE 13
VI pool performance targets for Drug Product A

| Quality Attribute | Performance Target |
|---|---|
| Purity by SE-UPLC (%) | ≥90.0 |
| HMW by SE-UPLC (%) | ≤5.5 |
| iCIEF Region 1 (%) | ≤55.0 |
| iCIEF Region 2 (%) | ≥25.0 |
| iCIEF Region 3 (%) | ≤30.0 |
| Purity by NR MCE (%) | ≥85.0 |
| LMW by NR MCE (%) | ≤7.50 |
| Purity by R MCE (%) | ≥90.0 |
| LMW by R MCE (%) | ≤7.50 |
| NGHC by R MCE (%) | ≤1.15 |

### 3.1.1. Purity by SE-UPLC

Equation 1 represents the regression curve developed as a predictive model for purity by SE-UPLC. This equation is based on the response surface methodology (RSM) calculated from purity by SE-UPLC observed at the conclusion of each production bioreactor evaluated in the DO excursion study.

Purity by SE-UPLC (%)=95.6 Equation 1. Predictive equation for factorial RSM(actual)—purity by SE-UPLC

Attempts to derive a statistical model describing purity by SE-UPLC have produced a model with a negative predicted R². This implies that the mean model, or the mean of the overall data set (95.6%), is a better predictor of process performance than the derived RSM. As such, the mean model has been selected to illustrate the relationship between the evaluated input parameters and purity by SE-UPLC. Selection of the mean model implies that none of the evaluated DO excursion input parameters are practically significant in determining purity by SE-UPLC within the evaluated parameter ranges.

Based on Equation 1, predicted values for purity by SE-UPLC for a single excursion within the entire design space meet the performance target of ≥90.0%.

### 3.1.2. HMW by SE-UPLC

Equation 2 represents the regression curve developed as a predictive model for HMW by SE-UPLC. This equation is based on the RSM calculated from HMW by SE-UPLC observed at the conclusion of each production bioreactor evaluated in the DO excursion study.

HMW by SE-UPLC (%)=4.3 Equation 2. Predictive equation for factorial model(actual)—HMW by SE-UPLC

Attempts to derive a statistical model describing HMW by SE-UPLC have produced a model with a negative predicted R². This implies the mean model, or the mean of the overall data set (4.3%) is a better predictor of process performance than the derived RSM. As such, the mean model has been selected to illustrate the relationship between the evaluated input parameters and HMW by SE-UPLC. Selection of the mean model implies none of the evaluated DO excursion input parameters are practically significant in determining HMW by SE-UPLC within the evaluated parameter ranges.

Based on Equation 2, predicted values for HMW by SE-UPLC for a single excursion within the entire design space meet the performance target of ≤5.5%.

3.1.3. iCIEF Region 1

A factorial RSM was developed using ANOVA techniques to describe the influence of each studied input parameter on iCIEF Region 1 observed at the conclusion of each production bioreactor. This derived response model is statistically significant (p-value<0.0001) with a non-significant lack of fit (p-value=0.2551).

Equation 3 describes the response surface for the iCIEF Region 1. This equation is based on the RSM calculated from iCIEF Region 1 observed at the conclusion of each production bioreactor evaluated in the DO excursion study.

iCIEF Region 1(%)=33.12919+(0.544916*A)+(−0.037803*B)+(0.132740*C)+(0.004669*D)+(−0.26111*AB)+(0.000917*CD) Equation 3. Predictive equation for factorial model (actual)—iCIEF Region 1

Table 14 summarizes terms identified as statistically significant (p-value<0.05) within this factorial RSM.

TABLE 14
Significant terms for the factorial RSM-iCIEF Region 1

| Main Effects | Interactions |
|---|---|
| DO Excursion Concentration (A) | DO Excursion Concentration * Excursion |
| Excursion Duration in the Early Phase (B) | Duration in the Middle Phase (AC) |
| Excursion Duration in the Middle Phase (C) | Excursion Duration in the Middle Phase * |
| Excursion Duration in the Late Phase (D) | Excursion Duration in the Late Phase (CD) |

Based on Equation 3, predicted values for iCIEF Region 1 for a single excursion within the entire design space meet the performance target of ≤55.0%.

3.1.4. iCIEF Region 2

A factorial RSM was further developed using ANOVA techniques to describe the influence of each studied input parameter on iCIEF Region 2 observed at the conclusion of the production bioreactor. This derived response model is statistically significant (p-value<0.0001) with a non-significant lack of fit (p-value=0.8788).

Equation 4 describes the response surface for the iCIEF Region 2. This equation is based on the RSM calculated from iCIEF Region 2 observed at the conclusion of each production bioreactor evaluated in the DO excursion study.

iCIEF Region 2(%)=52.80855+(−0.083138*A)+(−0.23228*B)+(−0.114596*C)+(−0.108988*D)+(0.14048*AC)+(+0.010721*AD)+(0.000608*BD) Equation 4. Predictive equation for factorial model (actual)—iCIEF Region 2

Table 15 summarizes terms identified as statistically significant (p-value<0.05) within this factorial RSM.

TABLE 15
Significant terms for the factorial RSM-iCIEF Region 2

| Main Effects | Interactions |
|---|---|
| DO Excursion Concentration (A) | DO Excursion Concentration * Excursion |
| Excursion Duration in the Early Phase (B) | Duration in the Middle Phase (AC) |
| Excursion Duration in the Middle Phase (C) | DO Excursion Concentration * Excursion |
| | Duration in the Late Phase (AD) |
| Excursion Duration in the Late Phase (D) | Excursion Duration in the Early Phase * |
| | Excursion Duration in the Late Phase (BD) |

Based on Equation 4, predicted values for iCIEF Region 2 for a single excursion within the entire design space meet the performance target of ≥25.0%.

3.1.5. iCIEF Region 3

A factorial RSM was also developed using ANOVA techniques to describe the influence of each studied input parameter on iCIEF Region 3 observed at the conclusion of the production bioreactor. This derived response model is statistically significant (p-value=0.0011) with a non-significant lack of fit (p-value=0.1303).

Equation 5 describes the response surface for the iCIEF Region 3. This equation is based on the RSM calculated from iCIEF Region 3 observed at the conclusion of each production bioreactor evaluated in the DO excursion study

iCIEF Region 3(%)=15.08326+(−0.403433*A)+(0.036138*B)+(0.025686*C)+(0.023799*D)+(−0.000750*CD) Equation 5. Predictive equation for factorial model (actual)—iCIEF Region 3

Table 16 summarizes terms identified as statistically significant (p-value<0.05) within this factorial RSM.

TABLE 16
Significant terms for the factorial RSM-iCIEF Region 3

| Main Effects | Interactions |
|---|---|
| DO Excursion Concentration (A) | Excursion Duration in the Middle Phase * |
| Excursion Duration in the Early Phase (B) | Excursion Duration in the Late Phase (CD) |
| Excursion Duration in the Middle Phase (C) | |
| Excursion Duration in the Late Phase (D) | |

Based on Equation 5, predicted values for iCIEF Region 3 for a single excursion within the entire design space meet the performance target of ≤30.0%.

### 3.1.6. Purity by NR MCE

A factorial RSM was developed using ANOVA techniques to describe the influence of each studied input parameter on purity by NR MCE observed at the conclusion of the production bioreactor. This derived response model is statistically significant (p-value<0.0001) with a significant lack of fit (p-value=0.0385).

Equation 6 describes the response surface for the purity by NR MCE. This equation is based on the RSM calculated from purity by NR MCE observed at the conclusion of each production bioreactor evaluated in the DO excursion study.

Purity by NR MCE (%)=91.38427+(0.311340*A)+(−0.016462*C)+(−0.20648*D) Equation 6. Predictive equation for factorial model (actual)—purity by NR MCE

Table 17 summarizes terms identified as statistically significant (p-value<0.05) within this factorial RSM.

TABLE 17
Significant terms for the factorial RSM-purity by NR MCE
Main Effects
DO Excursion Concentration (A)
Excursion Duration in the Middle Phase (C)
Excursion Duration in the Late Phase (D)

Based on Equation 6, predicted values for purity by NR MCE for a single excursion within the entire design space meet the performance target of ≥85.0%.

### 3.1.7. LMW by NR MCE

A factorial RSM was developed using ANOVA techniques to describe the influence of each studied input parameter on LMW by NR MCE observed at the conclusion of the production bioreactor. This derived response model is statistically significant (p-value=0.0002) with a non-significant lack of fit (p-value=0.0689).

Equation 7 describes the response surface for the LMW by NR MCE. This equation is based on the RSM calculated from LMW by NR MCE observed at the conclusion of each production bioreactor evaluated in the DO excursion study.

LMW by NR MCE (%)=6.76681+(−0.265920*A)+(0.014454*C)+(0.015729*D) Equation 7. Predictive equation for factorial model (actual)—LMW by NR MCE

Table 18 summarizes terms identified as statistically significant (p-value<0.05) within this factorial RSM.

TABLE 18
Significant terms for the factorial RSM - LMW by NR MCE
Main Effects

DO Excursion Concentration (A)
Excursion Duration in the Middle Phase (C)
Excursion Duration in the Late Phase (D)

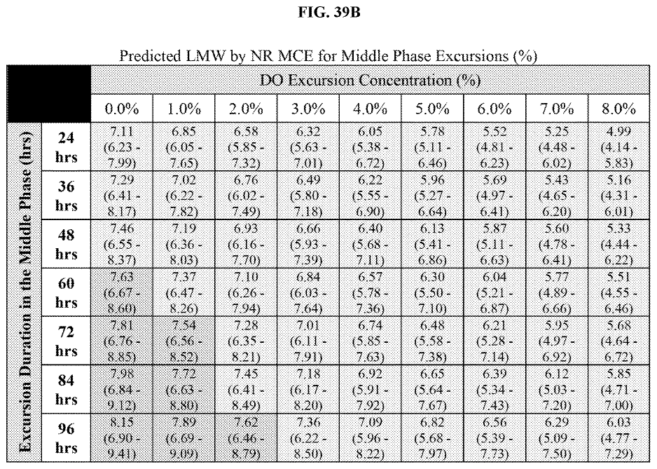

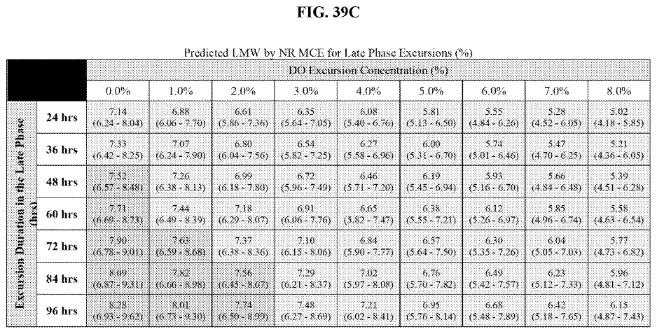

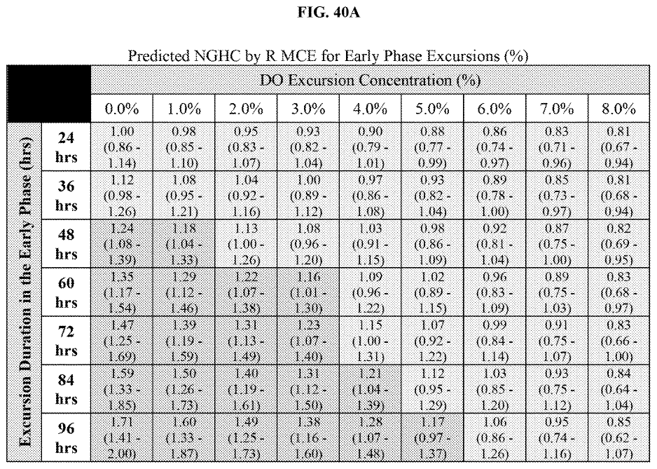

Based on Equation 6, not all predicted values for LMW by NR MCE for a single excursion within the entire design space meet the performance target of ≤7.50%. FIG. 39 shows an overview of predicted means (95% CI) for LMW by NR MCE within the design space, with FIG. 39A showing early phase excursions, FIG. 39B showing middle phase excursions, and FIG. 39C showing late phase excursions.

### 3.1.8. Purity by R MCE

A factorial RSM was developed using ANOVA techniques to describe influence of each studied input parameter on purity by R MCE observed at the conclusion of the production bioreactor. This derived response model is statistically significant (p-value=0.0018) with a non-significant lack of fit (p-value=0.2920).

Equation 8 describes the response surface for the purity by R MCE. This equation is based on the RSM calculated from purity by R MCE observed at the conclusion of each production bioreactor evaluated in the DO excursion study.

Purity by R MCE (%)=93.54198+(−0.007738*C)+(−0.008799*D) Equation 8. Predictive equation for factorial model (actual)—purity by R MCE

Table 19 summarizes terms identified as statistically significant (p-value<0.05) within this factorial RSM.

TABLE 19
Significant terms for the factorial RSM - purity by R MCE
Main Effects

Excursion Duration in the Middle Phase (C)
Excursion Duration in the Late Phase (D)

Based on Equation 8, predicted values for purity by R MCE for a single excursion within the entire design space meet the performance target of ≥90.0%.

### 3.1.9. LMW by R MCE

Equation 9 represents the regression curve developed as a predictive model for purity by LMW by R MCE. This equation is based on the response surface methodology (RSM) calculated from LMW by R MCE observed at the conclusion of each production bioreactor evaluated in the DO excursion study.

LMW by R MCE (%)=4.52 Equation 9. Predictive equation for factorial RSM (actual)—LMW by R MCE

Attempts to derive a statistical model describing LMW by R MCE have produced a model with a negative predicted R². This implies that the mean model, or the mean of the overall data set (4.52%) is a better predictor of process performance than the derived RSM. As such, the mean model has been selected to illustrate the relationship between the evaluated input parameters and LMW by R MCE. Selection of the mean model implies that none of the evaluated DO excursion input parameters are practically significant in determining LMW by R MCE within the evaluated parameter ranges.

Based on Equation 9, predicted values for LMW by R MCE for a single excursion within the entire design space meet the performance target of ≤7.50%.

### 3.1.10 NGHC by R MCE

A factorial RSM was developed using ANOVA techniques to describe the influence of each studied input parameter on NGHC by R MCE observed at the conclusion of the production bioreactor. This derived response model is statistically significant (p-value<0.0001) with a non-significant lack of fit (p-value=0.6974).

Equation 10 describes the response surface for the NGHC by R MCE. This equation is based on the RSM calculated from NGHC by R MCE observed at the conclusion of each production bioreactor evaluated in the DO excursion study.

NGHC by R MCE (%)=0.765668+(0.003540*A)+(0.009803*B)+(0.003041*C)+(0.003351*D)+(−0.001159*AB)+(−0.000063*BC) Equation 10. Predictive equation for factorial model (actual)—NGHC by R MCE

Table 20 summarizes terms identified as statistically significant (p-value<0.05) within this factorial RSM.

TABLE 20
Significant terms for the factorial RSM - NGHC by R MCE

| Main Effects | Interactions |
|---|---|
| DO Excursion Concentration (A) | DO Excursion Concentration * |
| Excursion Duration in the | Excursion Duration in the |
| Early Phase (B) | Early Phase (AB) |
| Excursion Duration in the | Excursion Duration in the |
| Middle Phase (C) | Early Phase * Excursion |
| Excursion Duration in the | Duration in the Middle |
| Late Phase (D) | Phase (BC) |

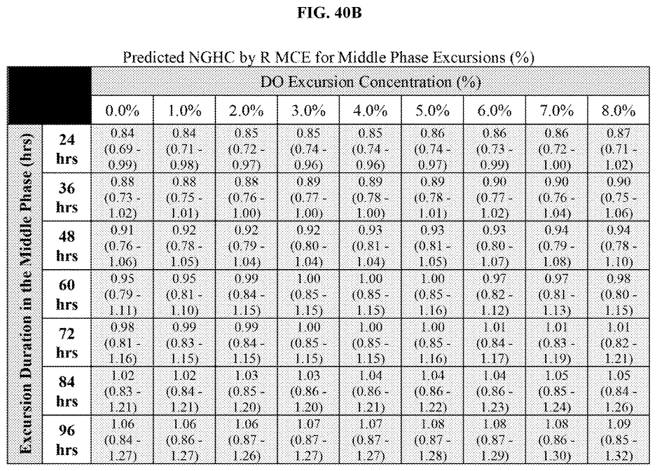

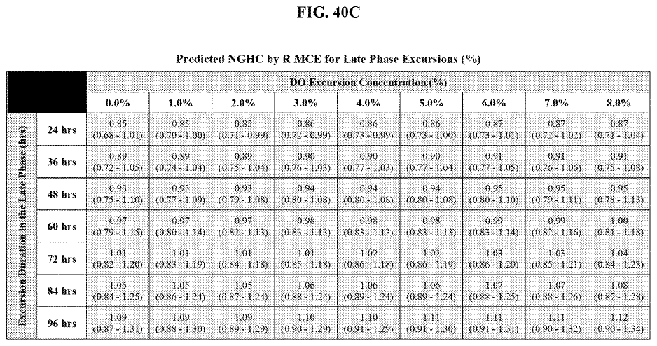

Based on Equation 10, not all predicted values for NGHC by R MCE for a single excursion within the entire design space meet the performance target of ≤1.15%. FIG. 40 shows an overview of predicted means (95% CI) for NGHC by R MCE within the design space, with FIG. 40A showing early phase excursions, FIG. 40B showing middle phase excursions, and FIG. 40C showing late phase excursions.

### 3.2. Dynamic Dissolved Oxygen Excursion Models for Drug Product B

Models for predicting product quality attributes based on dissolved oxygen excursions for a second drug product, Drug Product B, are set forth below. Drug product B has viral inactivation (VI) pool performance targets as outlined in Table 21.

TABLE 21
VI Pool Performance Targets for Drug Product B

| Quality Attribute | Performance Target |
|---|---|
| CHO HCP Content (ppm) | ≤1600 |
| Purity by R MCE (%) | ≥90.00 |
| NGHC by R MCE (%) | ≤8.40 |
| LMW by R MCE (%) | ≤2.80 |
| Purity by NR MCE (%) | ≥89.0 |
| LMW by NR MCE (%) | ≤5.8 |
| Purity by SE-UPLC (%) | ≥75.0 |
| HMW by SE-UPLC (%) | ≤22.5 |
| iCIEF Region 1 (%) | 20-45 |
| iCIEF Region 2 (%) | 50-65 |
| iCIEF Region 3 (%) | ≤8 |
| Average Fucosylated Glycans (%) | ≥80 |

### 3.2.1. CHO HCP Content

Equation 11 represents the regression curve developed as a predictive model for Chinese Hamster Ovary (CHO) host cell protein (HCP) content. This equation is based on the response surface methodology (RSM) calculated from CHO HCP content observed at the conclusion of each production bioreactor evaluated in the DO excursion study.

CHO HCP Content (ppm)=774.42 Equation 11. Predictive equation for CHO HCP content

Statistical analysis of the CHO HCP content data was unable to fit an acceptable response model. Therefore, the overall mean of the data points was identified as the appropriate predictor of response within the evaluated design space. This implies that no evaluated input parameters in their evaluated ranges were identified as statistically significant in determining the CHO HCP content response within the ranges applied during this study.

Based on Equation 11, predicted values for CHO HCP content for a single excursion within the entire design space meet the performance target of ≤1600 ppm.

### 3.2.2. Purity by R MCE

Equation 12 represents the regression curve developed as a predictive model for purity by R MCE. This equation is based on the response surface methodology (RSM) calculated from purity by R MCE observed at the conclusion of each production bioreactor evaluated in the DO excursion study.

Purity by R MCE (%)=93.42 Equation 12. Predictive equation for purity by R MCE

Statistical analysis of the purity by R MCE data was unable to fit an acceptable response model. Therefore, the overall mean of the data points was identified as the appropriate predictor of response within the evaluated design space. This implies that no evaluated input parameters in their evaluated ranges were identified as statistically significant in determining the purity by R MCE response within the ranges applied during this study.

Based on Equation 12, predicted values for purity by R MCE for a single excursion within the entire design space meet the performance target of ≥90.00%.

### 3.2.3. NGHC by R MCE

A factorial RSM was fit using a mixed-effects approach with restricted maximum likelihood (REML) to describe influence of each studied input parameter on NGHC by R MCE observed at the conclusion of each production bioreactor. Equation 13 describes the response surface for NGHC by R MCE. This equation is based on the RSM calculated from NGHC by R MCE observed at the conclusion of each production bioreactor evaluated in the DO excursion study.

$$Predicitive equation for NGHC by R MCE$$

$$6.2158367313 + 0.1815406263 \cdot ( \frac{( Excursion Duration - 26 )}{22} ) + Match ( Production Cell Culture Phase ) ( \begin{matrix}\begin{matrix}\begin{matrix}{}^{″} {Log}^{″} ⇒ 0.4501259042 \\ {}^{″} {Stationary}^{″} ⇒ - 0.222050976\end{matrix} \\ {}^{″} {Decline}^{″} ⇒ - 0.228074928\end{matrix} \\ else ⇒ .\end{matrix} ) + ( \frac{( Excursion Duration - 26 )}{22} ) \cdot Match ( Production Cell Culture Phase ) ( \begin{matrix}\begin{matrix}\begin{matrix}{}^{″} {Log}^{″} ⇒ 0.4909282094 \\ {}^{″} {Stationary}^{″} ⇒ - 0.248294764\end{matrix} \\ {}^{″} {Decline}^{″} ⇒ - 0.242633445\end{matrix} \\ else ⇒ .\end{matrix} ) . \qquad Equation 13$$

Table 22 summarizes terms identified as statistically significant (p-value<0.05) within this factorial RSM.

TABLE 22
Significant terms for the factorial RSM - NGHC by R MCE

| Main Effects | Interactions |
|---|---|
| Log Production Cell | Excursion Duration * Log |
| Culture Phase | Production Cell Culture Phase |

Based on Equation 13, predicted values for NGHC by R MCE for a single excursion within the entire design space meet the performance target of ≤8.40%. 3.2.4. LMW by R MCE

Equation 14 represents the regression curve developed as a predictive model for LMW by R MCE. This equation is based on the response surface methodology (RSM) calculated from purity by R MCE observed at the conclusion of each production bioreactor evaluated in the DO excursion study.

LMW by R MCE (%)=0.37 Equation 14. Predictive equation for LMW by R MCE

Statistical analysis of the LMW by R MCE data was unable to fit an acceptable response model. Therefore, the overall mean of the data points was identified as the appropriate predictor of response within the evaluated design space. This implies that no evaluated input parameters in their evaluated ranges were identified as statistically significant in determining the LMW by R MCE response within the ranges applied during this study.

Based on Equation 14, predicted values for LMW by R MCE for a single excursion within the entire design space meet the performance target of ≤2.80%.

### 3.2.5. Purity by NR MCE

A factorial RSM was fit using a mixed-effects approach with REML to describe influence of each studied input parameter on purity by NR MCE observed at the conclusion of each production bioreactor. Equation 15 describes the response surface for purity by NR MCE. This equation is based on the RSM calculated from purity by NR MCE observed at the conclusion of each production bioreactor evaluated in the DO excursion study.

$$Predictive equation for purity for NR MCE$$

$$95.31723774 + 0.1848556167 * ( \frac{( DO Setting - 3 )}{3} ) + Match ( Production Cell Culture Phase ) ( \begin{matrix}\begin{matrix}\begin{matrix}{}^{″} {Log}^{″} ⇒ 0.2044240193 \\ {}^{″} {Stationary}^{″} ⇒ - 0.412272552\end{matrix} \\ {}^{″} {Decline}^{″} ⇒ 0.2078485233\end{matrix} \\ else ⇒ .\end{matrix} ) . \qquad Equation 15$$

Table 23 summarizes terms identified as statistically significant (p-value<0.05) within this factorial RSM.

TABLE 23
Significant terms for the factorial RSM - purity by NR MCE
Main Effects

DO Setting
Stationary Production Cell Culture Phase
Decline Production Cell Culture Phase

Based on Equation 15, predicted values for purity by NR MCE for a single excursion within the entire design space meet the performance target of ≥89.0%. 3.2.6. LMW by NR MCE

A factorial RSM was fit using a mixed-effects approach with REML to describe influence of each studied input parameter on LMW by NR MCE observed at the conclusion of each production bioreactor. Equation 16 describes the response surface for LMW by NR MCE. This equation is based on the RSM calculated from LMW by NR MCE observed at the conclusion of each production bioreactor evaluated in the DO excursion study.

$$Predictive equation for LMW by NR MCE$$

$$4.3121781122 + - 0.193946708 \cdot ( \frac{( DO Setting - 3 )}{3} ) + 0.0743292446 \cdot ( \frac{( Excursion Duration - 26 )}{22} ) + Match ( Production Cell Culture Phase ) ( \begin{matrix}\begin{matrix}\begin{matrix}{}^{″} {Log}^{″} ⇒ - 0.136819276 \\ {}^{″} {Stationary}^{″} ⇒ 0.3165564502\end{matrix} \\ {}^{″} {Decline}^{″} ⇒ - 0.179737174\end{matrix} \\ else ⇒ .\end{matrix} ) + ( \frac{( DO Setting - 3 )}{3} ) \cdot ( ( \frac{( Excursion Duration - 26 )}{22} ) \cdot - 0.212474247 ) . \qquad Equation 16$$

Table 24 summarizes terms identified as statistically significant (p-value<0.05) within this factorial RSM.

TABLE 24
Significant terms for the factorial RSM - LMW by NR MCE

| Main Effects | Interactions |
|---|---|
| DO Setting | DO Setting * Excursion |
| Stationary Production Cell Culture Phase | Duration |

Based on Equation 16, predicted values for LMW by NR MCE for a single excursion within the entire design space meet the performance target of ≤5.8%.

### 3.2.7. Purity by SE-UPLC

A factorial RSM was fit using a mixed-effects approach with REML to describe influence of each studied input parameter on purity by SE-UPLC observed at the conclusion of each production bioreactor. Equation 17 describes the response surface for purity by SE-UPLC. This equation is based on the RSM calculated from purity by SE-UPLC observed at the conclusion of each production bioreactor evaluated in the DO excursion study.

$$Predictive equation for purity by SE - UPLC$$

$$90.168686314 + 1.1013970076 \cdot ( \frac{( DO Setting - 3 )}{3} ) + Match ( Production Cell Culture Phase ) ( \begin{matrix}\begin{matrix}\begin{matrix}{}^{″} {Log}^{″} ⇒ 0.9595771795 \\ {}^{″} {Stationary}^{″} ⇒ - 1.161280103\end{matrix} \\ {}^{″} {Decline}^{″} ⇒ 0.2017029239\end{matrix} \\ else ⇒ .\end{matrix} ) . \qquad Equation 17$$

Table 25 summarizes terms identified as statistically significant (p-value<0.05) within this factorial RSM.

TABLE 25
Significant terms for the factorial RSM - Purity by SE-UPLC
Main Effects

DO Setting
Log Production Cell Culture Phase
Stationary Production Cell Culture Phase

Based on Equation 17, predicted values for purity by SE-UPLC for a single excursion within the entire design space meet the performance target of ≥75.0%.

### 3.2.8. HMW by SE-UPLC

A factorial RSM was fit using a mixed-effects approach with REML to describe influence of each studied input parameter on HMW by SE-UPLC observed at the conclusion of each production bioreactor. Equation 18 describes the response surface for HMW by SE-UPLC. This equation is based on the RSM calculated from HMW by SE-UPLC observed at the conclusion of each production bioreactor evaluated in the DO excursion study.

$$Predictive equation for HMW by SE - UPLC$$

$$9.5431283566 + - 1.132159794 \cdot ( \frac{( DO Setting - 3 )}{3} ) + Match ( Production Cell Culture Phase ) ( \begin{matrix}\begin{matrix}\begin{matrix}{}^{″} {Log}^{″} ⇒ - 0.955235337 \\ {}^{″} {Stationary}^{″} ⇒ 1.1784371099\end{matrix} \\ {}^{″} {Decline}^{″} ⇒ - 0.223201773\end{matrix} \\ else ⇒ .\end{matrix} ) . \qquad Equation 18$$

Table 26 summarizes terms identified as statistically significant (p-value<0.05) within this factorial RSM.

TABLE 26
Significant terms for the factorial RSM - HMW by SE-UPLC
Main Effects

DO Setting
Log Production Cell Culture Phase
Stationary Production Cell Culture Phase

Based on Equation 18, predicted values for HMW by SE-UPLC for a single excursion within the entire design space meet the performance target of ≤22.5%.

3.2.9. iCIEF Region 1

A factorial RSM was fit using a mixed-effects approach with REML to describe influence of each studied input parameter on iCIEF Region 1 observed at the conclusion of each production bioreactor. Equation 19 describes the response surface for iCIEF Region 1. This equation is based on the RSM calculated from iCIEF Region 1 observed at the conclusion of each production bioreactor evaluated in the DO excursion study.

$$Predictive equation for iCIEF Region 1$$

$$34.637552154 + - 0.036161105 \cdot ( \frac{( DO Setting - 3 )}{3} ) + 0.0628260973 \cdot ( \frac{( Excursion Duration - 26 )}{22} ) + Match ( Production Cell Culture Phase ) ( \begin{matrix}\begin{matrix}\begin{matrix}{}^{″} {Log}^{″} ⇒ - 0.815851817 \\ {}^{″} {Stationary}^{″} ⇒ 0.4877268293\end{matrix} \\ {}^{″} {Decline}^{″} ⇒ 0.3281249879\end{matrix} \\ else ⇒ .\end{matrix} ) + ( \frac{( DO Setting - 3 )}{3} ) \cdot Match ( Production Cell Culture Phase ) ( \begin{matrix}\begin{matrix}\begin{matrix}{}^{″} {Log}^{″} ⇒ 0.7414332301 \\ {}^{″} {Stationary}^{″} ⇒ - 0.637077042\end{matrix} \\ {}^{″} {Decline}^{″} ⇒ - 0.104356189\end{matrix} \\ else ⇒ .\end{matrix} ) ( \frac{( Excursion Duration - 26 )}{22} ) + Match ( Production Cell Culture Phase ) ( \begin{matrix}\begin{matrix}\begin{matrix}{}^{″} {Log}^{″} ⇒ - 1.080458856 \\ {}^{″} {Stationary}^{″} ⇒ 1.0066598628\end{matrix} \\ {}^{″} {Decline}^{″} ⇒ 0.0737989927\end{matrix} \\ else ⇒ .\end{matrix} ) . \qquad Equation 19$$

Table 27 summarizes terms identified as statistically significant (p-value<0.05) within this factorial RSM.

TABLE 27
Significant terms for the factorial RSM - iCIEF Region 1

| Main Effects | Interactions |
|---|---|
| Log Production Cell | DO Setting * Log Production Cell |
| Culture Phase | Culture Phase |
| | DO Setting * Stationary Production |
| | Cell Culture Phase |
| Stationary Production | Excursion Duration * Log Production |
| Cell Culture Phase | Cell Culture Phase |
| | Excursion Duration * Stationary |
| | Production Cell Culture Phase |

Based on Equation 19, predicted values for iCIEF Region 1 for a single excursion within the entire design space meet the performance target of 20-45%.

3.2.10. iCIEF Region 2

A factorial RSM was fit using a mixed-effects approach with REML to describe influence of each studied input parameter on iCIEF Region 2 observed at the conclusion of each production bioreactor. Equation 20 describes the response surface for iCIEF Region 2. This equation is based on the RSM calculated from iCIEF Region 2 observed at the conclusion of each production bioreactor evaluated in the DO excursion study.

$$Predictive equation for iCIEF Region 2$$

$$58.92981185 + 0.6385144653 \cdot ( \frac{( DO Setting - 3 )}{3} ) + - 0.463703427 \cdot ( \frac{( Excursion Duration - 26 )}{22} ) + Match ( Production Cell Culture Phase ) ( \begin{matrix}\begin{matrix}\begin{matrix}{}^{″} {Log}^{″} ⇒ 0.4310906964 \\ {}^{″} {Stationary}^{″} ⇒ - 0.507820382\end{matrix} \\ {}^{″} {Decline}^{″} ⇒ 0.0767296861\end{matrix} \\ else ⇒ .\end{matrix} ) + ( \frac{( Excursion Duration - 26 )}{22} ) \cdot Match ( Production Cell Culture Phase ) ( \begin{matrix}\begin{matrix}\begin{matrix}{}^{″} {Log}^{″} ⇒ 0.3344302306 \\ {}^{″} {Stationary}^{″} ⇒ - 0.863337269\end{matrix} \\ {}^{″} {Decline}^{″} ⇒ 0.5289070383\end{matrix} \\ else ⇒ .\end{matrix} ) . \qquad Equation 20$$

Table 28 summarizes terms identified as statistically significant (p-value<0.05) within this factorial RSM.

TABLE 28
Significant terms for the factorial RSM - iCIEF Region 2

| Main Effects | Interactions |
|---|---|
| DO Setting | DO Setting * Stationary Production |
| Excursion Duration | Cell Culture Phase |

Based on Equation 20, predicted values for iCIEF Region 2 for a single excursion within the entire design space meet the performance target of 50-65%.

3.2.11. iCIEF Region 3

Equation 21 represents the regression curve developed as a predictive model for iCIEF Region 3. This equation is based on the response surface methodology (RSM) calculated from iCIEF Region 3 observed at the conclusion of each production bioreactor evaluated in the DO excursion study.

iCIEF Region 3(%)=6.47 Equation 21. Predictive equation for iCIEF Region 3

Statistical analysis of the iCIEF Region 3 data was unable to fit an acceptable response model. Therefore, the overall mean of the data points was identified as the appropriate predictor of response within the evaluated design space. This implies that no evaluated input parameters in their evaluated ranges were identified as statistically significant in determining the iCIEF Region 3 response within the ranges applied during this study.

Based on Equation 21, predicted values for iCIEF Region 3 for a single excursion within the entire design space meet the performance target of ≤8%.

### 3.2.12. Average Fucosylated Glycans

Equation 22 represents the regression curve developed as a predictive model for average fucosylated glycans. This equation is based on the response surface methodology (RSM) calculated from average fucosylated glycans observed at the conclusion of each production bioreactor evaluated in the DO excursion study.

Average Fucosylated Glycans (%)=89.76 Equation 22. Predictive equation for average fucosylated glycans

Statistical analysis of the average fucosylated glycans data was unable to fit an acceptable response model. Therefore, the overall mean of the data points was identified as the appropriate predictor of response within the evaluated design space. This implies that no evaluated input parameters in their evaluated ranges were identified as statistically significant in determining the average fucosylated glycans response within the ranges applied during this study.

Based on Equation 22, predicted values for average fucosylated glycans for a single excursion within the entire design space meet the performance target of ≥80%.

### 3.3. Case Study 1 (Drug Product A)

On production day 2 (early phase) of manufacturing Drug Product A, the DO concentration drops to 5.0%. After 24 hours, the DO returns to the normal set point. Table 29 shows the predicted data for quality attributes derived from the models outlined in Example 3.1.

TABLE 29
Point prediction for case study 1

| | Predicted | 95% CI Low | 95% CI High |
|---|---|---|---|
| Quality Attribute | Mean | for Mean | for Mean |
| Purity by SE-UPLC (%) | 95.6 | 94.9 | 96.4 |
| HMW by SE-UPLC (%) | 4.3 | 3.6 | 5.1 |
| iCIEF Region 1 (%) | 35.6 | 33.2 | 38.0 |
| iCIEF Region 2 (%) | 34.9 | 32.7 | 37.2 |
| iCIEF Region 3 (%) | 51.8 | 50.7 | 52.9 |
| Purity by NR MCE (%) | 92.9 | 92.1 | 93.8 |
| LMW by NR MCE (%) | 5.44 | 4.66 | 6.22 |
| Purity by R MCE (%) | 93.5 | 93.2 | 93.9 |
| LMW by R MCE (%) | 4.52 | 4.31 | 4.73 |
| NGHC by R MCE (%) | 0.88 | 0.77 | 0.99 |

Based on the predicted data shown in Table 29, the disclosed models predict, with 95% confidence, that all quality attributes will fall within their respective performance target, as set forth in Table 13. With this knowledge, it can be determined that the affected lot of Drug Product A should continue through the manufacturing process as normal.

### 3.4. Case Study 2 (Drug Product A)

On production day 6 (middle phase) of manufacturing Drug Product A, the DO concentration drops to 1.0%. After 84 hours, the DO returns to the normal set point. Table 30 shows the predicted data for quality attributes derived from the models outlined in Example 3.1.

TABLE 30
Point prediction for case study 2

| | Predicted | 95% CI Low | 95% CI High |
|---|---|---|---|
| Quality Attribute | Mean | for Mean | for Mean |
| Purity by SE-UPLC (%) | 95.6 | 94.9 | 96.4 |
| HMW by SE-UPLC (%) | 4.3 | 3.6 | 5.1 |
| iCIEF Region 1 (%) | 42.6 | 39.1 | 46.1 |
| iCIEF Region 2 (%) | 44.3 | 41.7 | 46.8 |
| iCIEF Region 3 (%) | 16.8 | 14.6 | 19.1 |
| Purity by NR MCE (%) | 90.3 | 89.1 | 91.5 |
| LMW by NR MCE (%) | 7.72 | 6.63 | 8.80 |
| Purity by R MCE (%) | 92.9 | 92.4 | 93.4 |
| LMW by R MCE (%) | 4.52 | 4.31 | 4.73 |
| NGHC by R MCE (%) | 1.02 | 0.84 | 1.21 |

Based on the predicted data shown in Table 30, the disclosed models predict, with 95% confidence, that there is high risk of exceeding the performance target for LMW by NR MCE and moderate risk of exceeding the performance target for NGHC by R MCE, as set forth in Table 13. With this knowledge, it can be determined that operations should stop and the affected lot of Drug Product A should be discarded without any further processing.

### 3.5. Case Study 3 (Drug Product B)

On production day 3 (log phase) of manufacturing Drug Product B, the DO concentration drops to 1.0%. After 32 hours, the DO returns to the normal set point. Table 31 shows the predicted data for quality attributes derived from the models outlined in Example 3.2.

TABLE 31
Point prediction for case study 3

| | Predicted | 95% CI Low | 95% CI High |
|---|---|---|---|
| Quality Attribute | Mean | for Mean | for Mean |
| CHO HCP Content (ppm) | 774.42 | 658.19 | 890.66 |
| Purity by R MCE (%) | 93.42 | 93.14 | 3.69 |
| NGHC by R MCE (%) | 6.85 | 6.38 | 7.32 |
| LMW by R MCE (%) | 0.39 | 0.29 | 0.48 |
| Purity by NR MCE (%) | 95.10 | 94.91 | 95.89 |
| LMW by NR MCE (%) | 4.36 | 4.01 | 4.72 |
| Purity by SE-UPLC (%) | 90.39 | 89.04 | 91.75 |
| HMW by SE-UPLC (%) | 9.34 | 7.96 | 10.73 |
| iCIEF Region 1 (%) | 33.07 | 32.21 | 33.94 |
| iCIEF Region 2 (%) | 58.90 | 57.89 | 59.91 |
| iCIEF Region 3 (%) | 6.52 | 6.00 | 7.04 |
| Average Fucosylated | 89.71 | 88.96 | 90.46 |
| Glycans (%) | | | |

Based on the predicted data shown in Table 31, the disclosed models predict, with 95% confidence, that all quality attributes will fall within their respective performance target, as set forth in Table 21. With this knowledge, it can be determined that the affected lot of Drug Product B should continue through the manufacturing process as normal.

### Example 4. Gene Expression Analysis for Predicting Process Performance

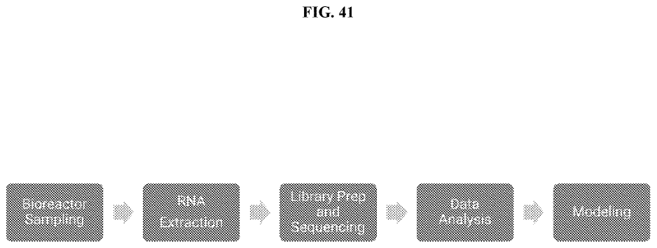

A complementary method for analysis of cell culture condition excursions during biological product production was developed on the basis of transcriptomic analysis. First, an analysis of gene expression levels in recombinant cells was undertaken to enable an understanding of how varying process parameters changes the transcriptomic profiles of the cells, and then models were developed using transcriptomic markers to predict parameters such as product quality. A general workflow for developing the disclosed method, including bioreactor sampling, RNA extraction, library preparation and sequencing, data analysis, and finally modeling, is illustrated in FIG. 41. Having alternative metrics for predicting batch outcomes after a DO excursion in addition to the direct DO excursion measurements of Examples 1-3 may be advantageous, for example, in the case of unreliable oxygen sensor data.

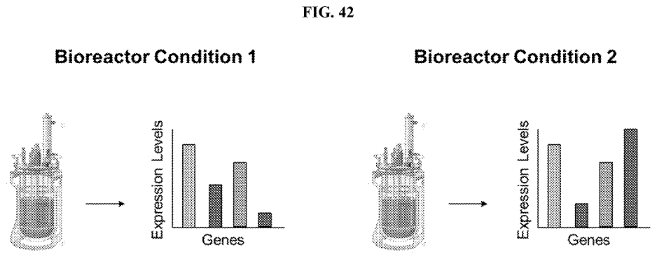

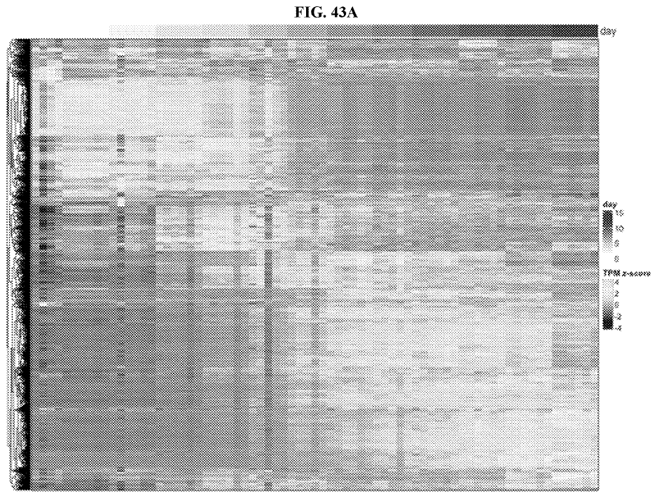

The batches of recombinant cells expressing mAb1 described in Table 4 were subjected to transcriptomic analysis either under normal conditions (center point analysis) or during dissolved oxygen excursions (excursion analysis) to develop a transcriptomic profile of cells based on cell culture condition, as illustrated in FIG. 42. Analytical techniques that were applied included the use of RNA sequencing (RNAseq) and microarrays. RNAseq involves high-throughput sequencing of all transcripts from a cell, whereas microarrays offer quantification of a set of predetermined sequences.

### 4.1. Transcriptomic Analysis of Control Production Batches

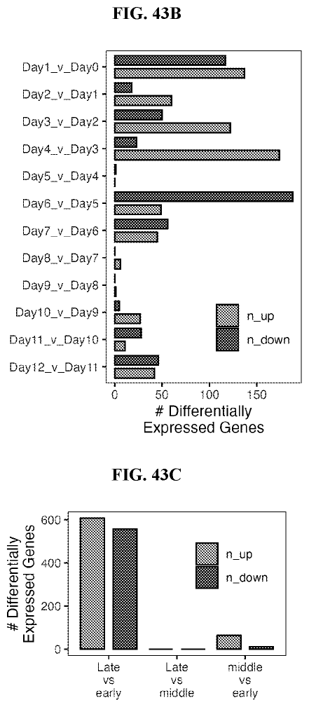

A heat map visualization of gene expression over time across cell batches under control conditions only is shown in FIG. 43A. Cell batches were sorted using hierarchical clustering, a method of cluster analysis that builds a hierarchy of clusters either by repeatedly merging smaller clusters into larger ones (agglomerative approach) or by dividing larger clusters into smaller ones (divisive approach). It uses a measure of distance between data points or clusters to decide how to merge or split them, and the results are often visualized as a dendrogram, which illustrates the arrangement of the clusters and their relationships.

Gene expression was found to be highly variable on day 0 of cell culture, and normalized by day 1, as shown in FIG. 43B. Expression patterns in the early phase of cell culture were markedly different than in the late phase, as shown in FIG. 43C. The largest source of gene expression differences was time, moreso than batch effects or where the samples were sequenced.

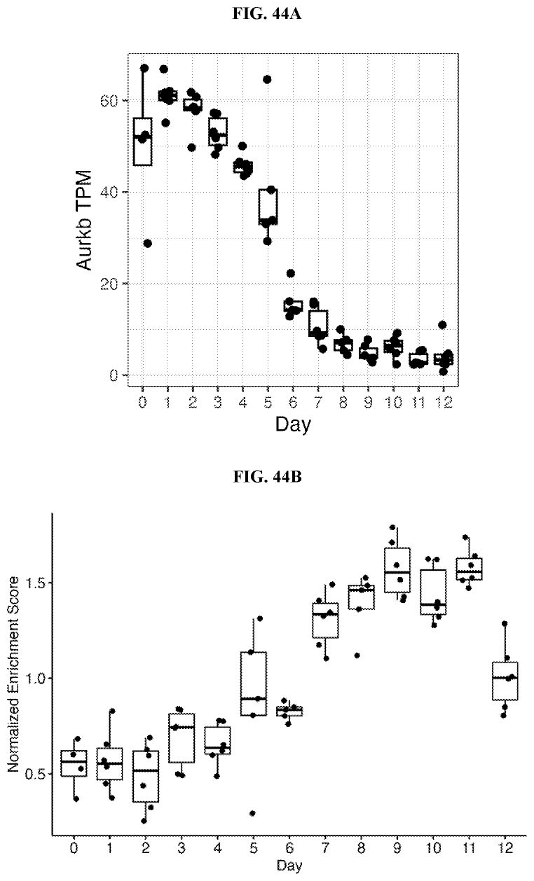

Genes with distinct dynamics over time in control bioreactors were individually assessed. For example, changes in expression of aurora kinase B (AURKB) in terms of transcripts per million (TPM) are shown in FIG. 44A. AURKB is a serine/threonine kinase that regulates key processes during mitosis, including chromatin condensation, spindle assembly, and cytokinesis. It is part of the chromosomal passenger complex and is essential for proper cell division. The high expression of AURKB during the highest period of cell division, in the early phase of cell culture, and low expression in the later phase of cell culture, validates the use of gene expression for characterizing cell culture status over time. In contrast, a group of genes that serve as a hallmark of hypoxia (“HALLMARK_HYPOXIA” gene set) were quantified over time in terms of normalized enrichment score, and their expression increased over time, as shown in FIG. 44B. This is expected behavior as cell culture growth results in increased oxygen consumption and thereby reduced dissolved oxygen in the cell culture, and validates both the measurement of gene expression over time and the importance of a control baseline for understanding changes in hypoxia-related gene expression.

Overall, significant changes in gene expression over time were observed, highlighting dynamic shifts across the transcriptome. Distinctive expression patterns of various genes were identified throughout production phases, revealing temporal trends in gene activity. Specific biological pathways were found to undergo alterations over time, providing insights into functional changes in cellular processes. A baseline of variability across the transcriptome was established, serving as a reference for understanding cellular dynamics and deviations in response to varying cell culture conditions.

### 4.2. Differentially Expressed Genes Related to Dissolved Oxygen Excursions

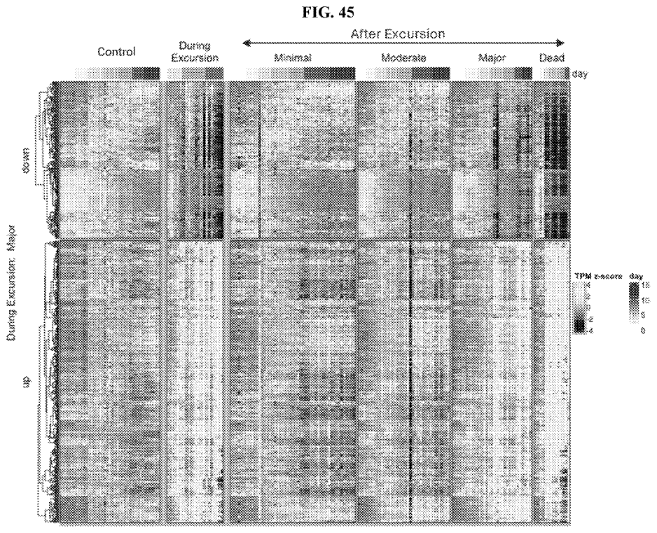

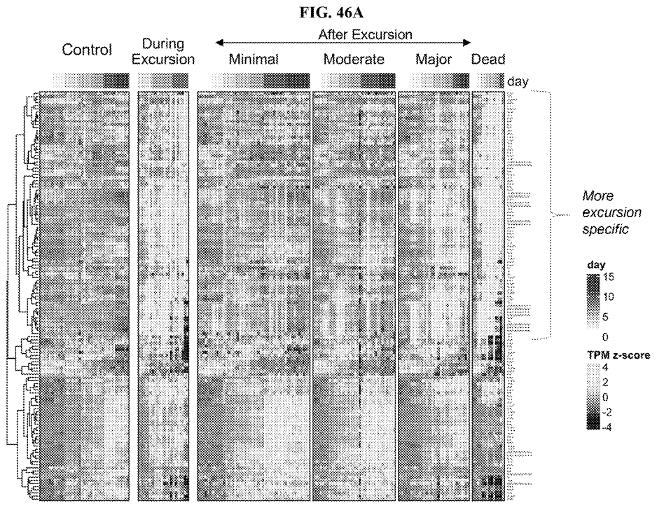

Gene expression changes were then assessed in response to dissolved oxygen excursions, both during and after the excursion. A heat map demonstrating differentially expressed genes (DEGs) under different conditions (control, minimal excursion, moderate excursion, major excursion, and critical excursion/dead) over time is shown in FIG. 45. It was discovered that excursions had clear effects on the expression of particular genes, many of which persisted after the end of the excursion. How well gene expression recovered after the end of the excursion was a significant indicator of batch performance, cell growth, and ultimately expressed protein titer. Major and dead samples maintained differential gene expression at higher levels.

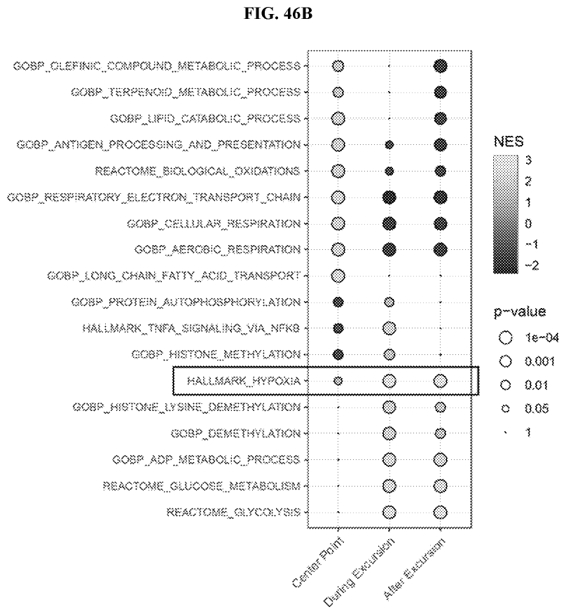

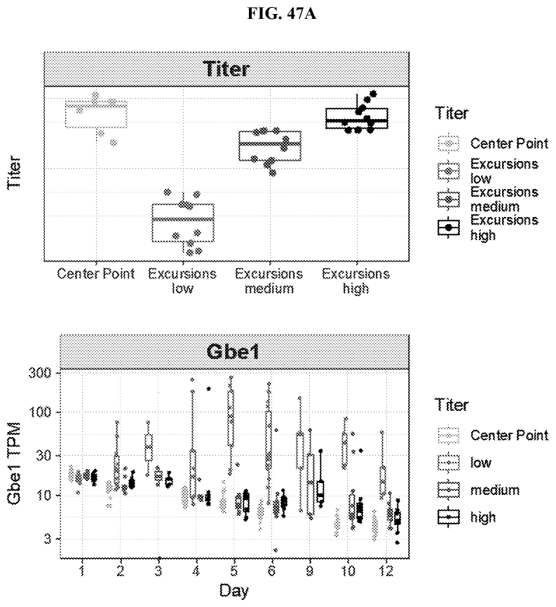

Differentially expressed genes were subjected to further analysis to identify pathways with distinct behavior between control batches and batches subjected to dissolved oxygen excursions. For example, hypoxia-related genes (“HALLMARK_HYPOXIA” gene set) were compared between control and excursion batches, during and after excursions, as shown in FIG. 46A. Changes in hypoxia-related genes are to be expected in response to a dissolved oxygen excursion, and confirm the relevance of the transcriptomic analysis to cell culture conditions. However, additional pathways with substantial changes in normalized expression scores (NES) were also identified, such as histone modification, as shown in FIG. 46B. The identification of affected pathways presents the potential for interventions to remedy the effects of excursions—for example, supplementing with lipids to help return the cells to baseline conditions—and, conversely, additional biomarkers for predicting that a batch will fail product quality limits and should be terminated in order to save time and resources.

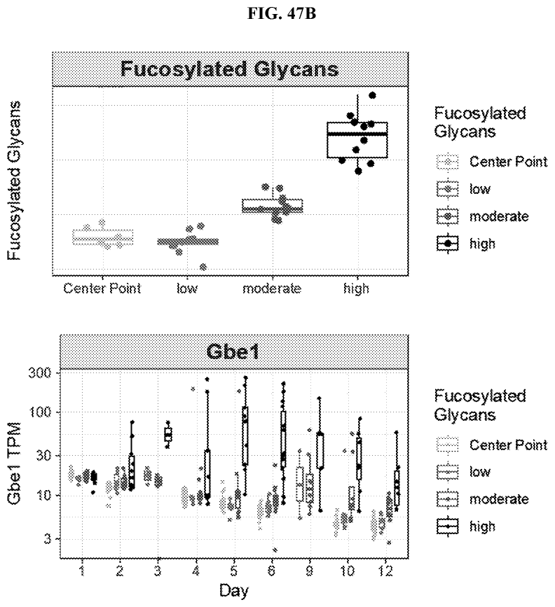

Transcriptomic analysis of dissolved oxygen-related DEGs allows for the identification of genes associated with critical quality attribute (CQA) deviations. For example, an analysis of expression of the gene encoding glycogen branching enzyme, GBE1, shows a correlation between GBE1 expression levels, even at an early stage, and final titer and fucosylated glycans. FIG. 47A shows a grouping of cell culture batches based on low, medium, and high titer, and the distribution of GBE1 expression levels across each titer group, with lower GBE1 expression correlating to higher titer. FIG. 47B shows a grouping of cell culture batches based on low, medium, and high fucosylated glycan levels, and the distribution of GBE1 expression levels across each fucosylated glycan group, with lower GBE1 expression correlating to lower fucosylated glycans. Based on this exemplary analysis, GBE1 expression at an early stage in a cell culture batch could be measured and the batch could be continued or discontinued based on whether the GBE1 expression level correlates to an acceptable titer and fucosylated glycan outcome.

Overall, different groups categorized by excursion severity (minimal, moderate, major, and critical/dead) exhibited distinct gene expression profiles. Several biological pathways were significantly impacted by reduced dissolved oxygen levels, including hypoxia-related and metabolic pathways influencing product quality. Larger gene expression changes were observed in batches from the “major” and “dead” groups, with these alterations persisting even after the excursions ended. Genes linked to product quality outcomes were identified, offering potential biomarkers for process optimization.

### 4.3. Identifying Differentially Expressed Genes Predictive of Product Quality

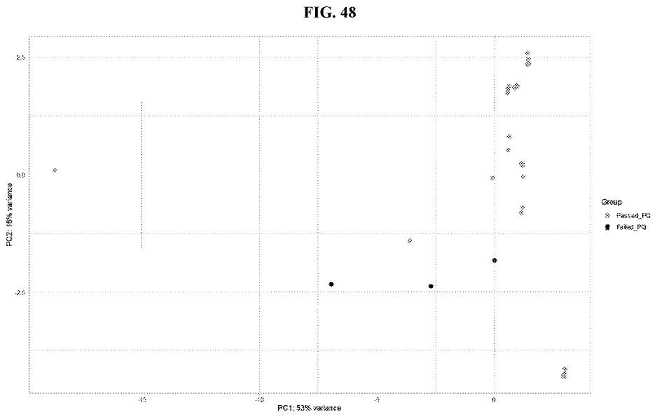

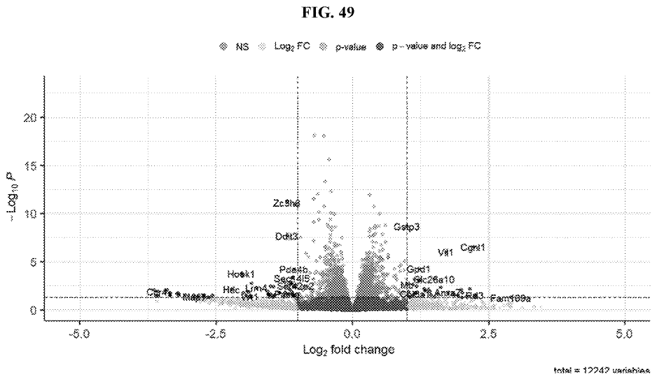

As an exemplary application of the disclosed methods, the collected transcriptomic data was leveraged in order to identify genes associated with product quality failure as early as day 4 of production. Batches were considered based on having early phase excursions, and samples were grouped based on passing or failing product quality specifications. Batches that had died entirely as a result of excursions were excluded from this analysis. Differential gene expression analysis was performed based only on gene expression at day 4, and the results were subjected to principal component analysis, as shown in FIG. 48. Principal component analysis (PCA) is used to linearize multi-dimensional data and group or separate samples as an indication of sample differences and outliers. Batches that failed product quality specifications formed a cluster compared to batches that passed product quality specifications, demonstrating that transcriptomic analysis as early as day 4 of production is indicative of later product quality and can be used as a predictive tool for biologics manufacturing.

The same day 4 data was visualized as a volcano plot to identify genes with the most statistically significant and largest magnitude differential expression between samples that passed versus failed product quality specifications, as shown in FIG. 49. Particular genes of interest were subjected to further analysis, with a comparison of transcripts per million for control batches, excursion batches that passed product quality specifications, and excursion batches that failed product quality specifications. An exemplary subset of these analyses are further described below.

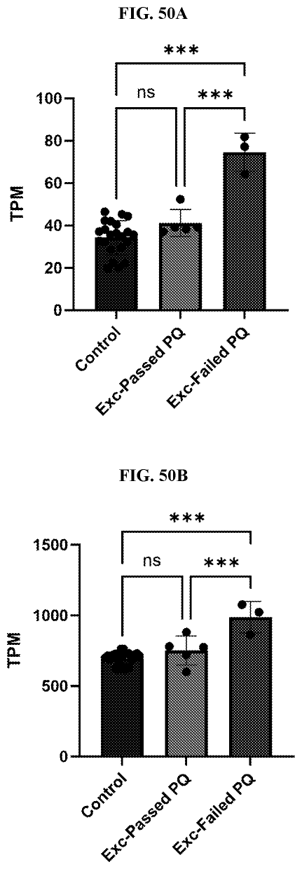

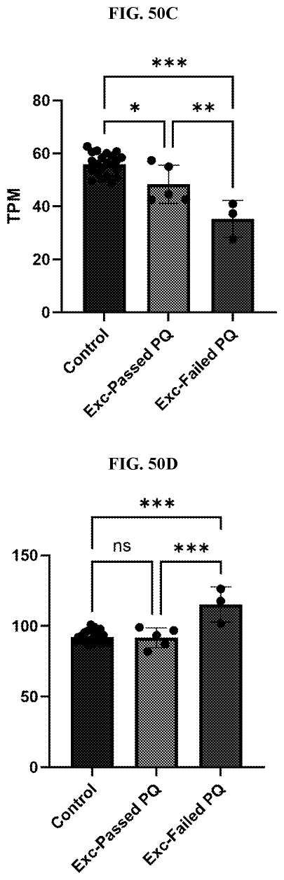

An analysis of glutathione S-transferase pi 3 (GSTP3) differential expression is shown in FIG. 50A, demonstrating a marked increase in expression specific to excursion batches that failed product quality specifications. The GSTP3 protein is part of the glutathione S-transferase family, which catalyzes the conjugation of glutathione to various harmful compounds. This helps neutralize toxins and protect cells from oxidative stress. GSTP3 is involved in cellular defense against damage caused by free radicals, carcinogens, and other toxic substances.

Differential expression of superoxide dismutase 1 (SOD1) is shown in FIG. 50B, with a significant increase in expression in excursion batches that failed product quality specifications. SOD1 encodes an enzyme that converts superoxide radicals (byproducts of oxygen metabolism) into hydrogen peroxide and oxygen, reducing oxidative stress. SOD1 protects cells from oxidative damage and is critical in high-energy tissues like the brain and muscles. Mutations in SOD1 are linked to amyotrophic lateral sclerosis (ALS).

Differential expression of solute carrier family 7 member 11 (SLC7A11) is shown in FIG. 50C, demonstrating decreased expression in excursion batches compared to control batches and a further decrease in batches that failed product quality specifications. SLC7A11 encodes a cystine/glutamate antiporter, which imports cystine into cells in exchange for glutamate. Cystine is used to synthesize glutathione, a key antioxidant. SLC7A11 maintains redox homeostasis and protects cells from ferroptosis, a type of cell death caused by lipid peroxidation. It is also implicated in cancer cell survival under oxidative stress.

Differential expression of phosphatidylinositol transfer protein membrane-associated 1 (PITPNM1) is shown in FIG. 50D, demonstrating a significant increase in expression in excursion batches that failed product quality specifications. The PITPNM1 protein is involved in lipid signaling and membrane trafficking. It regulates the transfer of phosphatidylinositol and other lipids between cellular compartments. PITPNM1 plays a role in maintaining lipid homeostasis, intracellular signaling, and vesicle transport. PITPNM1 has been associated with neurological function and may influence processes like synaptic signaling.

Therefore, the disclosed methods allow for the identification of biomarkers whose expression levels as early as day 4 are statistically significantly predictive of final product quality. These biomarkers can be used as early indicators for determining the outcome of a cell culture process far in advance of product quality testing, allowing for considerable savings in time and expense.

### 4.4. Identifying Differentially Expressed Genes Predictive of Final Viability

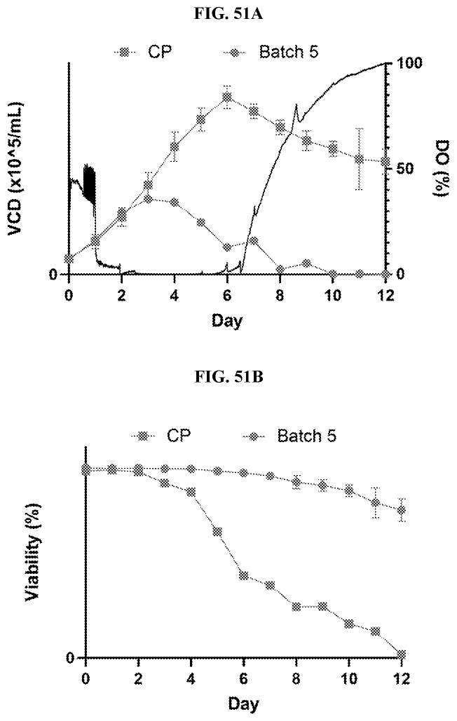

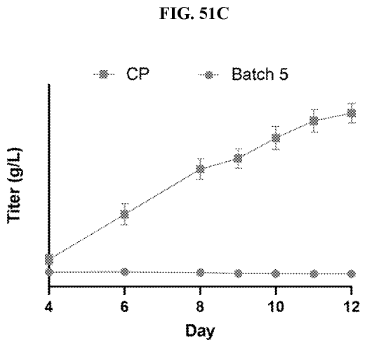

As another exemplary application of the disclosed transcriptomic methods, the collected transcriptomic data was used to identify genes associated with the final viability of a cell culture batch as early as day 4 of production. Batches that eventually died out as a result of excursions were compared against control batches and against excursion batches that had passed product quality testing. Day 4 data was subjected to differential gene expression analysis, principal component analysis and volcano plot analysis. Viable cell density, viability, and titer of an exemplary dead batch compared to a control batch is shown in FIG. 51A, FIG. 51B and FIG. 51C, respectively. At day 4, some differences in VCD and viability are apparent, but the full extent of the later viability issues in the dead batches is not yet clear. Therefore, methods for predicting a later decline in viability as early as day 4 would be useful for determining in advance whether a batch that has experienced a DO excursion should be allowed to proceed or should be terminated on the basis of viability.

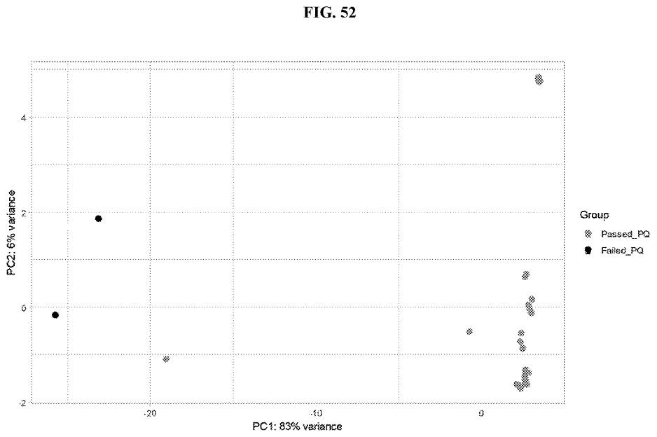

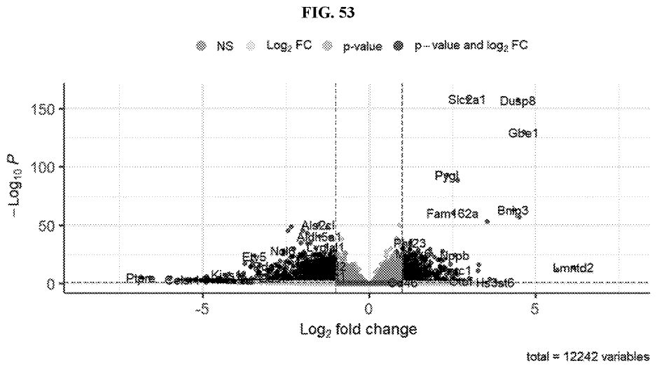

FIG. 52 shows a principal component analysis comparing day 4 gene expression differences between dead batches and excursion batches that passed product quality specifications. Dead batches form a cluster compared to successful batches, demonstrating that gene expression as early as day 4 of production can be predictive of later cell culture viability. The same data was further investigated using a volcano plot to identify genes with the most statistically significant and largest magnitude differential expression between dead batches and successful batches, as shown in FIG. 53. Identified genes were subjected to further analysis by comparing transcripts per million at day 4 between control batches, excursion batches that passed product quality testing, and dead batches, an exemplary subset of which are further described.

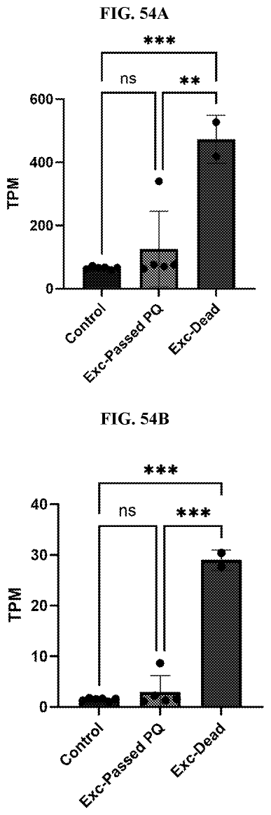

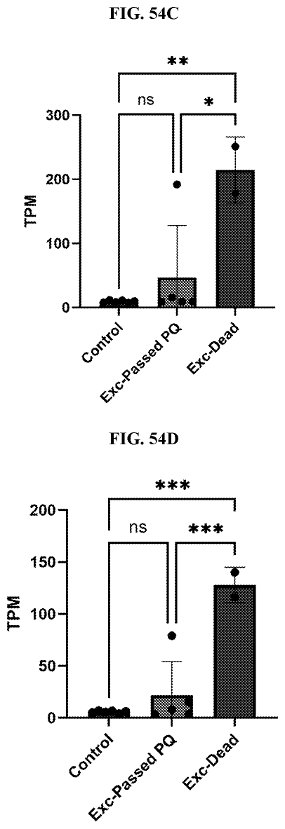

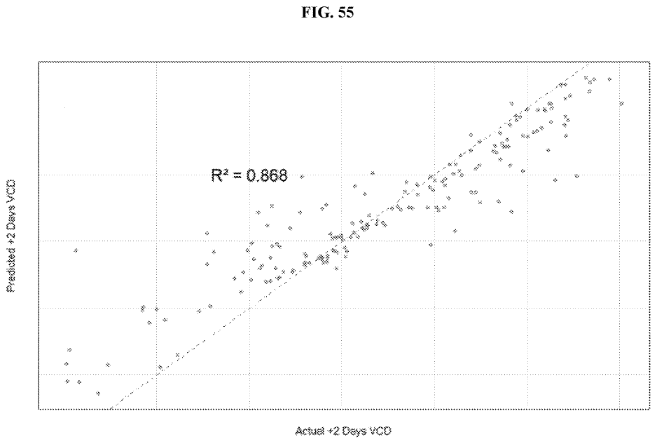

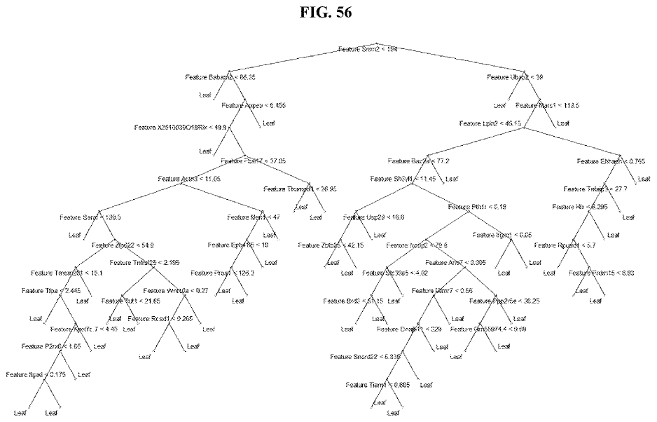

An analysis of solute carrier family 2 member 1 (SLC2A1) differential expression is shown in FIG. 54A, demonstrating a significant increase in expression in dead batches compared to control batches or excursion batches that passed product quality testing. SLC2A1 encodes GLUT1, a glucose transporter protein responsible for facilitating the transport of glucose across cell membranes. GLUT1 is critical for glucose uptake in tissues, especially those with high energy demands like the brain and red blood cells. Mutations in SLC2A1 are linked to Glut1 Deficiency Syndrome, which can cause seizures, developmental delay, and movement disorders.

Differential expression of dual specificity phosphatase 8 (DUSP8) is shown in FIG. 54B, demonstrating a significant increase in expression in dead batches compared to control batches or excursion batches that passed product quality testing. DUSP8 is part of the dual specificity phosphatase family, which regulates the activity of mitogen-activated protein kinases (MAPKs) by dephosphorylating them. It is involved in controlling cellular responses to stress, inflammation, and growth signals. DUSP8 specifically targets MAPKs like ERK, JNK, and p38, which are critical for processes such as apoptosis, cell proliferation, and immune responses.

Differential expression of glucan branching enzyme 1 (GBE1) is shown in FIG. 54C, demonstrating a significant increase in expression in dead batches compared to control batches or excursion batches that passed product quality testing. GBE1 encodes an enzyme responsible for glycogen branching during glycogen synthesis. It introduces α-1,6 glycosidic branches into glycogen, making it more soluble and functional. GBE1 is essential for normal glycogen storage and metabolism. Mutations in GBE1 are associated with Glycogen Storage Disease Type IV (Andersen Disease), which can lead to liver dysfunction, muscle weakness, and cardiomyopathy.

Differential expression of ankyrin repeat domain 37 (ANKRD37) is shown in FIG. 54D, demonstrating a significant increase in expression in dead batches compared to control batches or excursion batches that passed product quality testing. The ANKRD37 protein contains ankyrin repeat domains, which are involved in protein-protein interactions. ANKRD37 is thought to be involved in cellular stress responses, particularly hypoxia. It may play a role in regulating gene expression or protein stability under stress.

Therefore, the disclosed methods allow for the identification of biomarkers whose expression levels as early as day 4 are predictive of final cell culture batch viability. These biomarkers can be used as early indicators for determining the outcome of a cell culture process far in advance of cell culture harvesting, allowing for considerable savings in time and expense.

### 4.5. Using Random Forest Modeling to Predict Batch Performance

Random forest modeling was applied to correlate transcriptomic data collected from day 4 onward from the batches described in Table 4 to a prediction of final batch performance. Random forest modeling can include steps of data splitting and bootstrapping, building decision trees, aggregating predictions, evaluating model performance, and finally measuring feature importance, as further described below.

Random forest modeling begins by creating multiple subsets of the training data through a process called bootstrapping. This involves randomly sampling the data (with replacement), meaning some data points may be repeated while others are excluded from each subset. For each bootstrapped subset, a decision tree is built. However, instead of considering all features at every split, random forest involves selecting a random subset of features (a process called “feature bagging”) to reduce correlation between trees and improve diversity. Once all trees are built, the predictions are aggregated. For classification problems, majority voting is used, meaning that the class predicted most often by the trees is chosen. For regression problems, the average of all tree predictions is calculated.

The model's accuracy is assessed using metrics like accuracy (for classification) or mean squared error (for regression). Additionally, “out-of-bag” (OOB) error can be calculated using data points excluded from bootstrapping to evaluate performance without needing a separate validation set. Random forest modeling can also be used to measure feature importance by analyzing how much each feature contributes to reducing impurity in the trees. This helps identify the most relevant features for predictions.

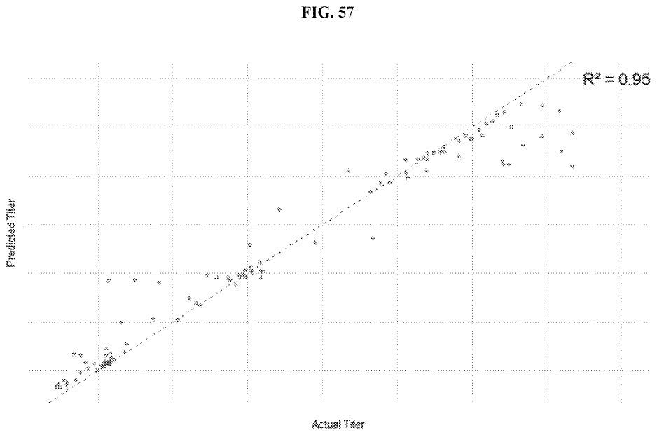

Random forest modeling was applied to the transcriptomic data to predict production batch performance based on gene expression at day 4 and later. The predicted viable cell density (VCD) at 2 days after gene expression measurement correlated with the actual VCD with an R² of 0.868, as shown in FIG. 55. An exemplary analysis of feature importance is shown in FIG. 56, with particular differentially expressed genes separated based on their importance to the random forest model prediction.

Random forest modeling was also applied to predict recombinant protein titer from the production batches, with an R² of 0.95 between the predicted and actual titer, as shown in FIG. 57. This demonstrates the usefulness of applying gene expression analysis for accurately predicting a final protein titer of a cell culture process.

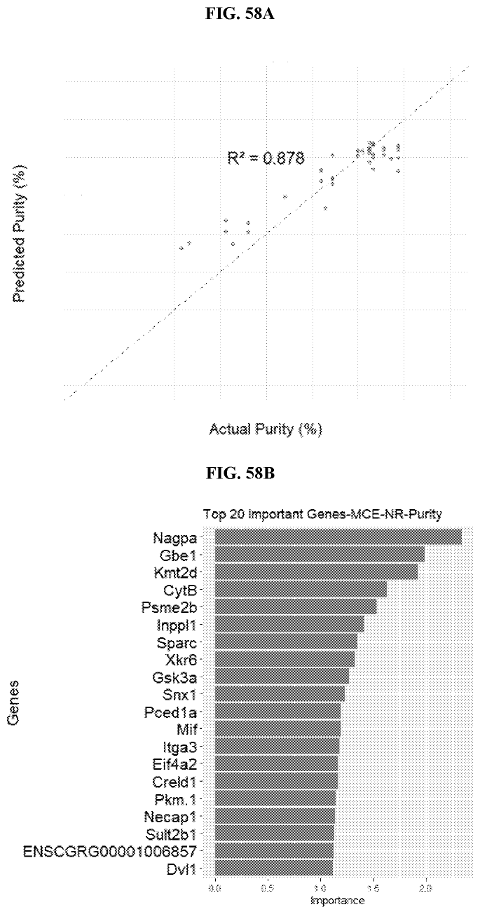

Random forest modeling was further applied to predict product quality outcome across a variety of attributes for production batches subjected to control conditions or dissolved oxygen excursions. For example, the model was applied to predict protein purity by non-reduced (NR) microfluidic capillary electrophoresis (MCE), with an R² of 0.878 between predicted and actual purity, as shown in FIG. 58A. Genes that were particularly important for predicting purity by NR MCE were identified using the same modeling system, as shown in FIG. 58B, allowing for the identification of biomarkers that can be used moving forward to quickly and easily predict product quality outcomes early on in a production batch.

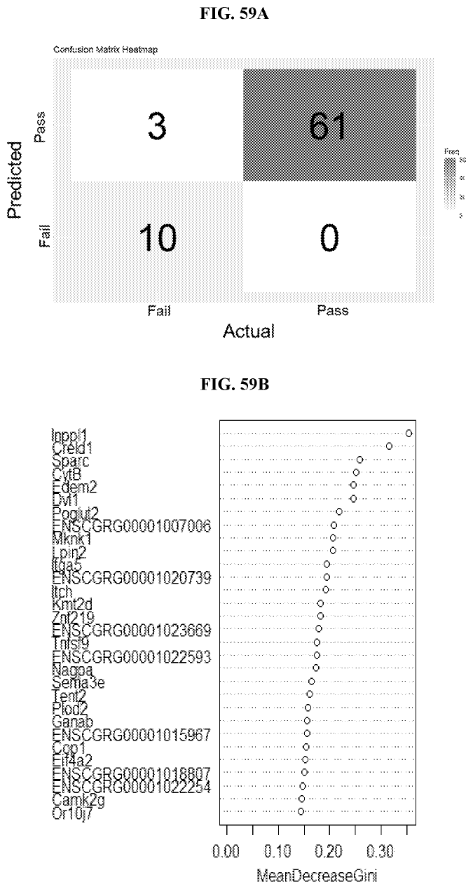

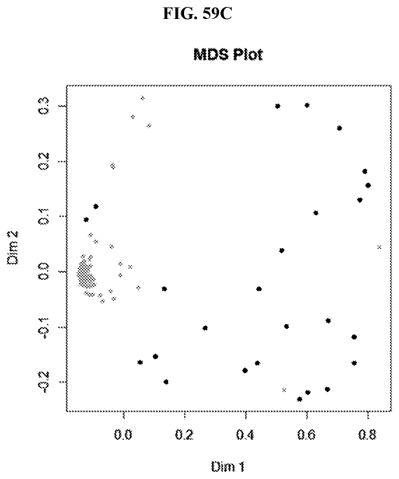

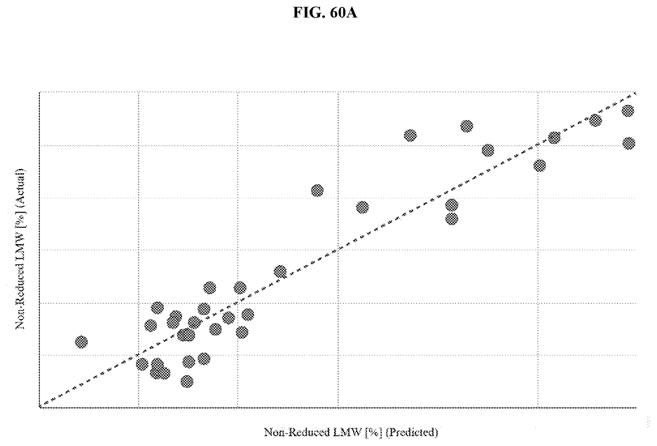

The overall accuracy of product quality outcome predictions was 96.0%, with a 95% confidence interval of 88.61%-99.16% and a p value of 0.0004742. A comparison of predicted passes and fails of product quality testing is illustrated in FIG. 59A. An analysis of feature importance is shown in FIG. 59B, demonstrating how random forest modeling can be used to identify genes of particular importance for predicting the outcome of a production batch, which can be used as biomarkers. A multidimensional scaling plot is shown in FIG. 59C, showing that failed batches and successful batches cluster separately based on the measured features, validating the predictive power of the transcriptomic dataset. The correlation of predicted versus actual product quality across various attributes is shown in Table 32 below, for random forest models based on transcriptomics data as described here as well as the regression models based on dissolved oxygen data as described in Example 1. Both models show a strong correlation across many product quality attributes and are useful for predicting final product quality.

TABLE 32
Random Forest Predictive Models for Product Quality Attributes

| | Random Forest | Regression |
|---|---|---|
| Attribute | Model R² | Model R² |
| Purity by NR MCE | 0.878 | 0.613 |
| LMW by NR MCE | 0.86 | 0.712 |
| Final titer | 0.853 | 0.819 |
| Fucosylated glycans | 0.647 | 0.9 |
| NGHC by R MCE | 0.626 | 0.54 |
| iCIEF Region 1 | 0.532 | 0.713 |
| iCIEF Region 2 | 0.407 | 0.802 |
| iCIEF Region 3 | 0.373 | 0.627 |
| HMW by SE-UPLC | 0.26 | N/A |

Overall, random forest modeling demonstrated reliable predictions of viable cell density (VCD) two days after gene expression measurements and of protein titer. Using gene expression profiles from day 4 onward, batch outcomes for product quality testing could be predicted with approximately 90% accuracy. Genes of particular importance for predicting the outcomes of production batches could be identified and used as biomarkers for efficient and reliable batch outcome predictions moving forward. This approach provides a robust framework for anticipating batch performance and identifying factors influencing product quality.

As with models derived from dissolved oxygen measurements as described in Examples 1-3, the use of models derived from gene expression measurements allows for determining, for example, whether a cell culture that has undergone an excursion from nominal conditions should undergo a modified manufacturing process, should be escalated in the quality system, or should be terminated. Instead of basing manufacturing decisions on the specifics of cell culture condition excursions, this method allows for using genetic biomarkers as early as day 4 of production to predict, for example, what a final product quality will be, and whether the product will pass or fail product quality specifications well in advance of the actual product testing. This method allows for online testing of in-process cell culture batches for reliably predicting an outcome of a production batch.

### Example 5. Multivariate Models for Predicting Product Quality

Multivariate models were produced using data from the transcriptomics study of Example 4 and from continuous cell culture health data for predicting product quality attributes of a recombinant protein. Online data was collected using a NOVA BioProfile® Flex Analyzer from the mAb1 production batches described in Table 4. Measured parameters included pH, partial pressure of oxygen (PO₂), partial pressure of carbon dioxide (PCO₂), glutamine (Gln), glutamate (Glu), glucose (Gluc), lactate (Lac), ammonium (NH₄+), sodium (Na+), potassium (K+), calcium (Ca++), phosphate, IgG, osmolality, total density, viable density, viability, average live diameter, vessel temperature, vessel pressure, sparging 02%, temperature-corrected pH (pH @ Temp), temperature-corrected partial pressure of oxygen (PO₂@Temp), temperature-corrected partial pressure of carbon dioxide (PCO₂@Temp), O₂ saturation, CO₂ saturation, and bicarbonate (HCO₃). It should be understood that any measurement of cell culture conditions could be included in this analysis, and can fall into the general term of, for example, “cell culture health parameter” used in this disclosure.

The cellular health data described above, the transcriptomics data described in Example 4, and product quality assay data were imported into SIMCA® multivariate data analysis software for further analysis. Batch evolution models (BEMs) were built for the cell culture health and transcriptomics data to produce a time series evolution of the input data. Batch level models (BLMs) were then built to predict product quality data based on either the cell culture health data or transcriptomics data. Each model was built for a specific product quality assay, for example iCIEF or MCE. Raw data were found to be best suited for product quality attribute prediction.

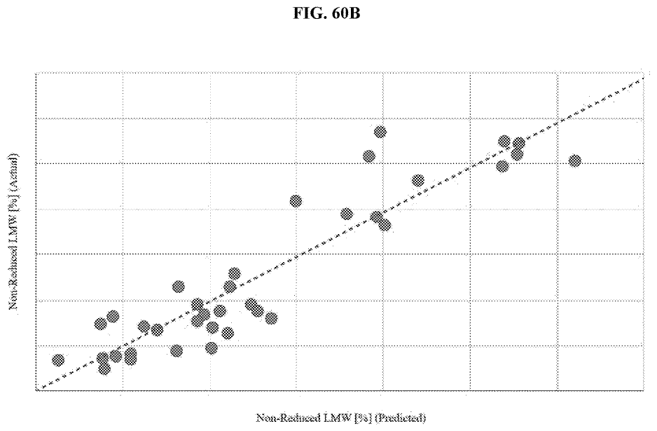

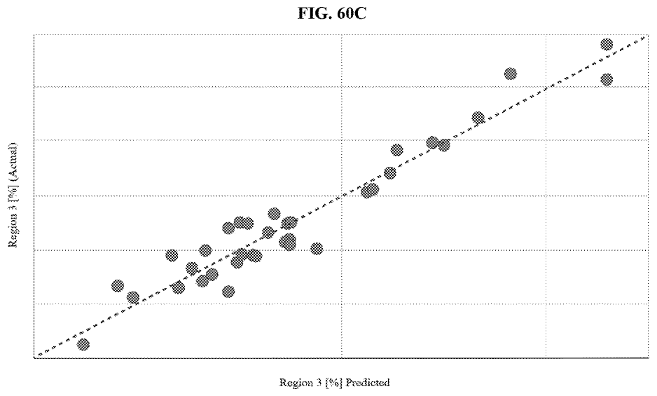

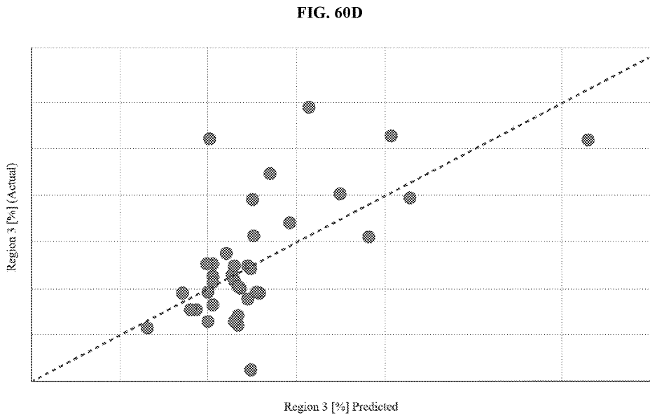

Predicted attributes were compared against observed attributes for each of the inputs. For example, FIG. 60A shows the observed versus predicted LMW by NR MCE using cell culture health data, which yielded a model with an R² of 0.8749. FIG. 60B shows the same prediction using transcriptomics data, which produced a model with an R² of 0.848.

FIG. 60C shows the observed versus predicted iCIEF Region 3 percentage using cell culture health data, with an R² of 0.9286. FIG. 60D shows the same prediction using transcriptomics data, with an R² of 0.5059.

Using multivariate modeling based on cell culture health data or transcriptomic data allowed for predicting individual product quality attributes of a recombinant protein product across production batches. This approach, for example using batch evolution models to produce a time series of cell culture data and batch level models to predict final product quality based on the cell culture data, provides a powerful tool for improving predictions of product quality.

This disclosure sets forth methods for determining a cell culture process based on predicting an effect of a process excursion on a cell culture outcome or recombinant product outcome. In particular, the Examples set forth the use of models correlating the properties of a dissolved oxygen excursion, gene expression, or cell culture health parameters to resulting impacts on product quality and cell culture batch outcomes. The Examples further set forth the use of predicted product quality and cell culture batch outcomes for controlling a manufacturing process of a recombinant protein, such as determining that a cell culture batch should be discontinued if a measured DO excursion, expression levels of biomarkers of interest, or cell culture health parameters are predicted to result in a product that falls outside of specification limits. This method results in significant improvements over conventional methods, which do not assess the effects of transient excursions on a cell culture process, do not correlate the properties of a DO excursion to an impact on product quality, do not correlate the expression of biomarker genes to final cell culture batch outcomes in response to a DO excursion, do not correlate cell culture health parameters to final cell culture batch outcomes in response to a DO excursion, and require time- and resource-intensive investigation of a product when a batch experiences a DO excursion.

The present invention is not to be limited in scope by the specific aspects described herein. Indeed, various modifications of the invention in addition to those described herein will become apparent to those skilled in the art from the foregoing description. Such modifications are intended to fall within the scope of the appended claims.

### ENUMERATED EXAMPLES

The following Enumerated Examples set forth below provide additional aspects of the present disclosure.
1. A method for controlling the duration of a cell culture batch, comprising:
   (a) culturing cells in a cell culture, wherein said cells produce a recombinant protein and said cell culture undergoes at least one dissolved oxygen (DO) excursion;
   (b) correlating said at least one DO excursion to at least one predicted product quality using at least one regression model; and
   (c) controlling the duration of said cell culture batch based on said at least one predicted product quality.
2. The method of Enumerated Example 1, wherein said controlling further comprises comparing each of said at least one predicted product qualities to a performance target for said product quality.
3. The method of Enumerated Example 1 or Enumerated Example 2, wherein said controlling further comprises terminating said cell culture batch when said at least one predicted product quality fails to meet said performance target.
4. The method of any one of Enumerated Examples 1-3, wherein said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.
5. The method of Enumerated Example 4, wherein said at least one product quality comprises protein titer and a performance target for protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.
6. The method of Enumerated Example 4 or Enumerated Example 5, wherein said at least one product quality comprises protein purity and a performance target for protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%.
7. The method of Enumerated Example 6, wherein said protein purity is measured by liquid chromatography, capillary electrophoresis, gel electrophoresis, or mass spectrometry.
8. The method of any one of Enumerated Examples 4-7, wherein said at least one product quality comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
9. The method of Enumerated Example 8, wherein said high molecular weight species are measured by liquid chromatography, capillary electrophoresis or gel electrophoresis.
10. The method of any one of Enumerated Examples 4-9, wherein said at least one product quality comprises low molecular weight species and a performance target for said lower molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
11. The method of Enumerated Example 10, wherein said low molecular weight species are measured by liquid chromatography, capillary electrophoresis or gel electrophoresis.
12. The method of any one of Enumerated Examples 4-11, wherein said at least one product quality comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%.
13. The method of Enumerated Example 12, wherein said non-glycosylated heavy chain is measured by reduced capillary electrophoresis.
14. The method of any one of Enumerated Examples 4-13, wherein said at least one product quality comprises charge variant profile and a performance target for said charge variant profile is an upper limit for a charge variant of about 50%, about 45%, about 40%, about 35%, about 30%, about 25%, about 20%, or about 15%.
15. The method of Enumerated Example 14, wherein said charge variant profile is measured by imaged capillary isoelectric focusing.
16. The method of any one of Enumerated Examples 4-15, wherein said at least one product quality comprises bispecific purity and a performance target for said bispecific purity is a lower limit of about 90%, about 91%, about 92%, about 93%, about 94%, about 95%, about 96%, about 97%, about 98%, or about 99%.
17. The method of Enumerated Example 16, wherein said bispecific purity is measured by protein A chromatography or mixed mode chromatography.
18. The method of any one of Enumerated Examples 4-17, wherein said at least one product quality comprises binding impurity and a performance target for said binding impurity is an upper limit of about 10%, about 9%, about 8%, about 7%, about 6%, about 5%, about 4%, about 3%, about 2%, or about 1%.
19. The method of Enumerated Example 18, wherein said binding impurity is measured by protein A chromatography or mixed mode chromatography.
20. The method of any one of Enumerated Examples 4-19, wherein said at least one product quality comprises non-binding impurity and a performance target for said non-binding impurity is an upper limit of about 10%, about 9%, about 8%, about 7%, about 6%, about 5%, about 4%, about 3%, about 2%, or about 1%.
21. The method of Enumerated Example 20, wherein said non-binding impurity is measured by protein A chromatography or mixed mode chromatography.
22. The method of any one of Enumerated Examples 1-21, wherein said cells are selected from the group consisting of CHO cells, HEK293 cells, and BHK cells.
23. The method of Enumerated Example 22, wherein said CHO cells are selected from the group consisting of CHO-K1, CHO DUX B-11, Veggie-CHO, GS-CHO, S-CHO, or CHO lec.
24. The method of any one of Enumerated Examples 1-23, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.
25. The method of any one of Enumerated Examples 1-24, wherein said recombinant protein is dupilumab.
26. The method of any one of Enumerated Examples 1-25, wherein said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.
27. The method of any one of Enumerated Examples 1-26, wherein said at least one DO excursion has a duration of less than 360 hours, from 240 hours to 360 hours, from 24 hours to 260 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.
28. The method of any one of Enumerated Examples 1-27, wherein said at least one DO excursion occurs during an early phase, a middle phase, and/or a late phase of said cell culture.
29. The method of any one of Enumerated Examples 1-28, wherein said at least one regression model comprises a random forest model, a batch evolution model and/or a batch level model.
30. A method for modifying a cell culture process, comprising:
   (a) culturing cells in a cell culture according to a first cell culture process, wherein said cells produce a recombinant protein and said cell culture undergoes at least one DO excursion;
   (b) predicting at least one recombinant protein parameter by using at least one regression model correlating said at least one DO excursion to said at least one recombinant protein parameter; and
   (c) culturing said cells according to a second cell culture process, wherein said second cell culture process is modified from said first cell culture process using said at least one predicted recombinant protein parameter.
31. The method of Enumerated Example 30, wherein modifying said first cell culture process comprises modifying a feed schedule, modifying a feed quantity, modifying an agitation rate, modifying a sparging rate, modifying a DO setpoint, and/or modifying a cell culture batch duration.
32. The method of Enumerated Example 30 or Enumerated Example 31, further comprising comparing each of said at least one recombinant protein parameters to a performance target for said recombinant protein parameter.
33. The method of any one of Enumerated Examples 30-32, wherein said at least one recombinant protein parameter is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.
34. The method of Enumerated Example 33, wherein said at least one recombinant protein parameter comprises protein titer and a performance target for said protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.
35. The method of Enumerated Example 33 or Enumerated Example 34, wherein said at least one recombinant protein parameter comprises protein purity and a performance target for said protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%.
36. The method of Enumerated Example 35, wherein said protein purity is measured by liquid chromatography, capillary electrophoresis, gel electrophoresis, or mass spectrometry.
37. The method of any one of Enumerated Examples 33-36, wherein said at least one recombinant protein parameter comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
38. The method of Enumerated Example 37, wherein said high molecular weight species are measured by liquid chromatography, capillary electrophoresis or gel electrophoresis.
39. The method of any one of Enumerated Examples 33-38, wherein said at least one recombinant protein parameter comprises low molecular weight species and a performance target for said low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
40. The method of Enumerated Example 39, wherein said low molecular weight species are measured by liquid chromatography, capillary electrophoresis or gel electrophoresis.
41. The method of any one of Enumerated Examples 33-40, wherein said at least one recombinant protein parameter comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%.
42. The method of Enumerated Example 41, wherein said non-glycosylated heavy chain is measured by reduced capillary electrophoresis.
43. The method of any one of Enumerated Examples 33-42, wherein said at least one recombinant protein parameter comprises charge variant profile and a performance target for said recombinant protein parameter is an upper limit for a charge variant of about 50%, about 45%, about 40%, about 35%, about 30%, about 25%, about 20%, or about 15%.
44. The method of Enumerated Example 43, wherein said charge variant profile is measured by imaged capillary isoelectric focusing.
45. The method of any one of Enumerated Examples 33-44, wherein said at least one recombinant protein parameter comprises bispecific purity and a performance target for said bispecific purity is a lower limit of about 90%, about 91%, about 92%, about 93%, about 94%, about 95%, about 96%, about 97%, about 98%, or about 99%.
46. The method of Enumerated Example 45, wherein said bispecific purity is measured by protein A chromatography or mixed mode chromatography.
47. The method of any one of Enumerated Examples 33-46, wherein said at least one recombinant protein parameter comprises binding impurity and a performance target for said binding impurity is an upper limit of about 10%, about 9%, about 8%, about 7%, about 6%, about 5%, about 4%, about 3%, about 2%, or about 1%.
48. The method of Enumerated Example 47, wherein said binding impurity is measured by protein A chromatography or mixed mode chromatography.
49. The method of any one of Enumerated Examples 33-48, wherein said at least one recombinant protein parameter comprises non-binding impurity and a performance target for said non-binding impurity is an upper limit of about 10%, about 9%, about 8%, about 7%, about 6%, about 5%, about 4%, about 3%, about 2%, or about 1%.
50. The method of Enumerated Example 49, wherein said non-binding impurity is measured by protein A chromatography or mixed mode chromatography.
51. The method of any one of Enumerated Examples 30-50, wherein said cells are selected from the group consisting of CHO cells, HEK293 cells, and BHK cells.
52. The method of Enumerated Example 51, wherein said CHO cells are selected from the group consisting of CHO-K1, CHO DUX B-11, Veggie-CHO, GS-CHO, S-CHO, or CHO lec.
53. The method of any one of Enumerated Examples 30-52, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.
54. The method of any one of Enumerated Examples 30-53, wherein said recombinant protein is dupilumab.
55. The method of any one of Enumerated Examples 30-54, wherein said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.
56. The method of any one of Enumerated Examples 30-55, wherein said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.
57. The method of any one of Enumerated Examples 30-56, wherein said at least one DO excursion occurs during an early phase, a middle phase, and/or a late phase of said cell culture.
58. The method of any one of Enumerated Examples 30-57, wherein said at least one regression model comprises a random forest model, a batch evolution model and/or a batch level model.
59. A system for improved production of a recombinant protein, said system comprising:
   (a) one or more vessels for culturing cells expressing a recombinant protein;
   (b) one or more probes for measuring one or more cell culture conditions in said one or more vessels;
   (c) one or more modules for controlling one or more settings in said one or more vessels;
   (d) a computing system coupled to said one or more probes and said one or more modules;
   (e) a prediction model stored in non-transitory machine-readable medium coupled to said computing system; and
   (f) a signal generator for controlling said one or more modules.
60. The system of Enumerated Example 59, wherein said one or more cell culture conditions are selected from a group consisting of dissolved oxygen levels, dissolved carbon dioxide levels, viable cell density, capacitance, pH, temperature, agitation rate, or sparging rate.
61. The system of Enumerated Example 59 or Enumerated Example 60, wherein said one or more settings are selected from a group consisting of pH, temperature, agitation rates, sparging rates, nutrient feeds, fluid control valves, and/or batch duration.
62. The system of any one of Enumerated Examples 59-61, further comprising one or more detectors for characterizing protein titer and/or product quality attributes.
63. The system of Enumerated Example 62, wherein said characterizing comprises imaged capillary isoelectric focusing (iCIEF), capillary electrophoresis (CE), microfluidic capillary electrophoresis (MCE), size exclusion chromatography (SEC), size exclusion-ultra-performance liquid chromatography (SE-UPLC), high-performance liquid chromatography (HPLC), mass spectrometry (MS), tandem mass spectrometry (MS/MS), liquid chromatography-mass spectrometry (LC-MS), capillary electrophoresis-mass spectrometry (CE-MS), gel electrophoresis, nuclear magnetic resonance, and/or surface plasmon resonance.
64. The system of any one of Enumerated Examples 59-63, further comprising a prediction model generator for generating a prediction model, comprising:
   (a) one or more vessels for culturing cells expressing a recombinant protein;
   (b) one or more probes for measuring one or more cell culture conditions in said one or more vessels;
   (c) one or more detectors for characterizing protein titer and/or product quality attributes of said recombinant protein;
   (d) a computing system for receiving data input from said one or more probes and one or more detectors; and
   (e) instructions stored in non-transitory machine-readable medium that when processed by said computing system perform operations comprising:
      (i) correlating data inputs from said one or more probes with data inputs from said one or more detectors;
      (ii) generating a prediction model of protein titer and/or product quality attributes based on said correlation(s); and
      (iii) storing said prediction model in a database.
65. The system of Enumerated Example 64, wherein said operations further comprise:
   (iv) determining one or more settings for said one or more vessels using said prediction model; and
   (v) storing said determined setting(s), said data inputs from said one or more probes and said data inputs from said one or more detectors in said database.
66. The system of Enumerated Example 64 or Enumerated Example 65, wherein said data input from said one or more probes of said prediction model generator includes a duration of at least one excursion of said one or more cell culture conditions, a magnitude of at least one excursion of said one or more cell culture conditions, and/or a timing of at least one excursion of said one or more cell culture conditions.
67. The system of Enumerated Example 66, wherein said timing is selected from a group consisting of day 0, day 1, day 2, day 3, day 4, day 5, day 6, day 7, day 8, day 9, day 10, day 11, day 12, day 13, day 14, and/or day 15 of said cell culture.
68. The system of Enumerated Example 66, wherein said timing is selected from a group consisting of an early phase, a middle phase, and/or a late phase of said cell culture.
69. The system of any one of Enumerated Examples 64-68, wherein said one or more cell culture conditions of said prediction model generator are selected from a group consisting of dissolved oxygen, dissolved carbon dioxide, viable cell density, capacitance, pH, temperature, agitation rate, and/or sparging rate.
70. The system of any one of Enumerated Examples 65-69, wherein said one or more settings of said prediction model generator are selected from a group consisting of pH, temperature, agitation rates, sparging rates, nutrient feeds, fluid control valves, and/or batch duration.
71. The system of any one of Enumerated Examples 64-70, wherein said operations of said prediction model generator further comprise comparing said protein titer and/or said product quality attributes to a performance target, wherein said performance target is based on values set forth in good manufacturing practices (GMP) or regulatory standards.
72. The system of any one of Enumerated Examples 59-71, further comprising a manual override disabling said signal generator.
73. The system of any one of Enumerated Examples 59-72, further comprising a report generator for generating a report from said prediction model.
74. The system of any one of Enumerated Examples 61-73, wherein said fluid control valves control transfer of said cells from one vessel to another vessel.
75. The system of any one of Enumerated Examples 59-74, wherein said prediction model comprises a random forest model, a batch evolution model and/or a batch level model.
76. A method for controlling the duration of a cell culture batch, comprising:
   (a) culturing cells in a cell culture, wherein said cells produce a recombinant protein and said cell culture undergoes at least one dissolved oxygen (DO) excursion;
   (b) correlating the expression of at least one gene in said cells to at least one predicted product quality using at least one regression model; and
   (c) controlling the duration of said cell culture batch based on said at least one predicted product quality.
77. The method of Enumerated Example 76, wherein said controlling further comprises comparing each of said at least one predicted product qualities to a performance target for said product quality.
78. The method of Enumerated Example 76 or Enumerated Example 77, wherein said controlling further comprises terminating said cell culture batch when said at least one predicted product quality fails to meet said performance target.
79. The method of any one of Enumerated Examples 76-78, wherein said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.
80. The method of Enumerated Example 79, wherein said at least one product quality comprises protein titer and a performance target for protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.
81. The method of Enumerated Example 79 or Enumerated Example 80, wherein said at least one product quality comprises protein purity and a performance target for protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%.
82. The method of Enumerated Example 81, wherein said protein purity is measured by liquid chromatography, capillary electrophoresis, gel electrophoresis, or mass spectrometry.
83. The method of any one of Enumerated Examples 79-82, wherein said at least one product quality comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
84. The method of Enumerated Example 83, wherein said high molecular weight species are measured by liquid chromatography, capillary electrophoresis or gel electrophoresis.
85. The method of any one of Enumerated Examples 79-84, wherein said at least one product quality comprises low molecular weight species and a performance target for said lower molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
86. The method of Enumerated Example 85, wherein said low molecular weight species are measured by liquid chromatography, capillary electrophoresis or gel electrophoresis.
87. The method of any one of Enumerated Examples 79-86, wherein said at least one product quality comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%.
88. The method of Enumerated Example 87, wherein said non-glycosylated heavy chain is measured by reduced capillary electrophoresis.
89. The method of any one of Enumerated Examples 79-88, wherein said at least one product quality comprises charge variant profile and a performance target for said charge variant profile is an upper limit for a charge variant of about 50%, about 45%, about 40%, about 35%, about 30%, about 25%, about 20%, or about 15%.
90. The method of Enumerated Example 89, wherein said charge variant profile is measured by imaged capillary isoelectric focusing.
91. The method of any one of Enumerated Examples 79-90, wherein said at least one product quality comprises bispecific purity and a performance target for said bispecific purity is a lower limit of about 90%, about 91%, about 92%, about 93%, about 94%, about 95%, about 96%, about 97%, about 98%, or about 99%.
92. The method of Enumerated Example 91, wherein said bispecific purity is measured by protein A chromatography or mixed mode chromatography.
93. The method of any one of Enumerated Examples 79-92, wherein said at least one product quality comprises binding impurity and a performance target for said binding impurity is an upper limit of about 10%, about 9%, about 8%, about 7%, about 6%, about 5%, about 4%, about 3%, about 2%, or about 1%.
94. The method of Enumerated Example 93, wherein said binding impurity is measured by protein A chromatography or mixed mode chromatography.
95. The method of any one of Enumerated Examples 79-94, wherein said at least one product quality comprises non-binding impurity and a performance target for said non-binding impurity is an upper limit of about 10%, about 9%, about 8%, about 7%, about 6%, about 5%, about 4%, about 3%, about 2%, or about 1%.
96. The method of Enumerated Example 95, wherein said non-binding impurity is measured by protein A chromatography or mixed mode chromatography.
97. The method of any one of Enumerated Examples 76-96, wherein said cells are selected from the group consisting of CHO cells, HEK293 cells, and BHK cells.
98. The method of Enumerated Example 97, wherein said CHO cells are selected from the group consisting of CHO-K1, CHO DUX B-11, Veggie-CHO, GS-CHO, S-CHO, or CHO lec.
99. The method of any one of Enumerated Examples 76-98, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.
100. The method of any one of Enumerated Examples 76-99, wherein said recombinant protein is dupilumab.
101. The method of any one of Enumerated Examples 76-100, wherein said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.
102. The method of any one of Enumerated Examples 76-101, wherein said at least one DO excursion has a duration of less than 360 hours, from 240 hours to 360 hours, from 24 hours to 260 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.
103. The method of any one of Enumerated Examples 76-102, wherein said at least one DO excursion occurs during an early phase, a middle phase, and/or a late phase of said cell culture.
104. The method of any one of Enumerated Examples 76-103, wherein said at least one regression model comprises a random forest model, a batch evolution model and/or a batch level model.
105. The method of any one of Enumerated Examples 76-104, wherein the expression of said at least one gene is measured using RNA sequencing (RNAseq), quantitative polymerase chain reaction (qPCR), or a microarray.
106. The method of any one of Enumerated Examples 76-105, wherein said at least one gene is a hypoxia-related gene.
107. A method for modifying a cell culture process, comprising:
   (a) culturing cells in a cell culture according to a first cell culture process, wherein said cells produce a recombinant protein and said cell culture undergoes at least one DO excursion;
   (b) predicting at least one recombinant protein parameter by using at least one regression model correlating the expression of at least one gene in said cells to said at least one recombinant protein parameter; and
   (c) culturing said cells according to a second cell culture process, wherein said second cell culture process is modified from said first cell culture process using said at least one predicted recombinant protein parameter.
108. The method of Enumerated Example 107, wherein modifying said first cell culture process comprises modifying a feed schedule, modifying a feed quantity, modifying an agitation rate, modifying a sparging rate, modifying a DO setpoint, and/or modifying a cell culture batch duration.
109. The method of Enumerated Example 107 or Enumerated Example 108, further comprising comparing each of said at least one recombinant protein parameters to a performance target for said recombinant protein parameter.
110. The method of any one of Enumerated Examples 107-109, wherein said at least one recombinant protein parameter is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.
111. The method of Enumerated Example 110, wherein said at least one recombinant protein parameter comprises protein titer and a performance target for said protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.
112. The method of Enumerated Example 110 or Enumerated Example 111, wherein said at least one recombinant protein parameter comprises protein purity and a performance target for said protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%.
113. The method of Enumerated Example 112, wherein said protein purity is measured by liquid chromatography, capillary electrophoresis, gel electrophoresis, or mass spectrometry.
114. The method of any one of Enumerated Examples 110-113, wherein said at least one recombinant protein parameter comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
115. The method of Enumerated Example 114, wherein said high molecular weight species are measured by liquid chromatography, capillary electrophoresis or gel electrophoresis.
116. The method of any one of Enumerated Examples 110-115, wherein said at least one recombinant protein parameter comprises low molecular weight species and a performance target for said low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
117. The method of Enumerated Example 116, wherein said low molecular weight species are measured by liquid chromatography, capillary electrophoresis or gel electrophoresis.
118. The method of any one of Enumerated Examples 110-117, wherein said at least one recombinant protein parameter comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%.
119. The method of Enumerated Example 118, wherein said non-glycosylated heavy chain is measured by reduced capillary electrophoresis.
120. The method of any one of Enumerated Examples 110-119, wherein said at least one recombinant protein parameter comprises charge variant profile and a performance target for said recombinant protein parameter is an upper limit for a charge variant of about 50%, about 45%, about 40%, about 35%, about 30%, about 25%, about 20%, or about 15%.
121. The method of Enumerated Example 120, wherein said charge variant profile is measured by imaged capillary isoelectric focusing.
122. The method of any one of Enumerated Examples 110-121, wherein said at least one recombinant protein parameter comprises bispecific purity and a performance target for said bispecific purity is a lower limit of about 90%, about 91%, about 92%, about 93%, about 94%, about 95%, about 96%, about 97%, about 98%, or about 99%.
123. The method of Enumerated Example 122, wherein said bispecific purity is measured by protein A chromatography or mixed mode chromatography.
124. The method of any one of Enumerated Examples 110-123, wherein said at least one recombinant protein parameter comprises binding impurity and a performance target for said binding impurity is an upper limit of about 10%, about 9%, about 8%, about 7%, about 6%, about 5%, about 4%, about 3%, about 2%, or about 1%.
125. The method of Enumerated Example 124, wherein said binding impurity is measured by protein A chromatography or mixed mode chromatography.
126. The method of any one of Enumerated Examples 110-125, wherein said at least one recombinant protein parameter comprises non-binding impurity and a performance target for said non-binding impurity is an upper limit of about 10%, about 9%, about 8%, about 7%, about 6%, about 5%, about 4%, about 3%, about 2%, or about 1%.
127. The method of Enumerated Example 126, wherein said non-binding impurity is measured by protein A chromatography or mixed mode chromatography.
128. The method of any one of Enumerated Examples 107-127, wherein said cells are selected from the group consisting of CHO cells, HEK293 cells, and BHK cells.
129. The method of Enumerated Example 128, wherein said CHO cells are selected from the group consisting of CHO-K1, CHO DUX B-11, Veggie-CHO, GS-CHO, S-CHO, or CHO lec.
130. The method of any one of Enumerated Examples 107-129, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.
131. The method of any one of Enumerated Examples 107-130, wherein said recombinant protein is dupilumab.
132. The method of any one of Enumerated Examples 107-131, wherein said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.
133. The method of any one of Enumerated Examples 107-132, wherein said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.
134. The method of any one of Enumerated Examples 107-133, wherein said at least one DO excursion occurs during an early phase, a middle phase, and/or a late phase of said cell culture.
135. The method of any one of Enumerated Examples 107-134, wherein said at least one regression model comprises a random forest model, a batch evolution model and/or a batch level model.
136. The method of any one of Enumerated Examples 107-135, wherein the expression of said at least one gene is measured using RNAseq, qPCR, or a microarray.
137. The method of any one of Enumerated Examples 107-136, wherein said at least one gene is a hypoxia-related gene.
138. A system for improved production of a recombinant protein, said system comprising:
   (a) one or more vessels for culturing cells expressing a recombinant protein;
   (b) one or more ports for collecting one or more samples from said one or more vessels; (c) one or more analyzers for analyzing said one or more samples;
   (d) one or more modules for controlling one or more settings in said one or more vessels;
   (e) a computing system coupled to said one or more analyzers and said one or more modules;
   (f) a prediction model stored in non-transitory machine-readable medium coupled to said computing system; and
   (g) a signal generator for controlling said one or more modules.
139. The system of Enumerated Example 138, wherein said one or more analyzers are configured to analyze gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O2%, temperature-corrected pH, temperature-corrected partial pressure of oxygen, temperature-corrected partial pressure of carbon dioxide, O2 saturation, CO2 saturation, and/or bicarbonate.
140. The system of Enumerated Example 138 or Enumerated Example 139, wherein said one or more analyzers are coupled online to said one or more ports.
141. The system of any one of Enumerated Examples 138-140, wherein said one or more settings are selected from a group consisting of pH, temperature, agitation rates, sparging rates, nutrient feeds, fluid control valves, and/or batch duration.
142. The system of any one of Enumerated Examples 138-141, further comprising one or more detectors for characterizing protein titer and/or product quality attributes.
143. The system of Enumerated Example 142, wherein said characterizing comprises imaged capillary isoelectric focusing (iCIEF), capillary electrophoresis (CE), microfluidic capillary electrophoresis (MCE), size exclusion chromatography (SEC), size exclusion-ultra-performance liquid chromatography (SE-UPLC), high-performance liquid chromatography (HPLC), mass spectrometry (MS), tandem mass spectrometry (MS/MS), liquid chromatography-mass spectrometry (LC-MS), capillary electrophoresis-mass spectrometry (CE-MS), gel electrophoresis, nuclear magnetic resonance, and/or surface plasmon resonance.
144. The system of any one of Enumerated Examples 138-143, further comprising a prediction model generator for generating a prediction model, comprising:
   (a) one or more vessels for culturing cells expressing a recombinant protein;
   (b) one or more ports for collecting one or more samples from said one or more vessels;
   (c) one or more analyzers for analyzing said one or more samples;
   (d) one or more detectors for characterizing protein titer and/or product quality attributes of said recombinant protein;
   (e) a computing system for receiving data input from said one or more analyzers and one or more detectors; and
   (f) instructions stored in non-transitory machine-readable medium that when processed by said computing system perform operations comprising:
      (i) correlating data inputs from said one or more analyzers with data inputs from said one or more detectors;
      (ii) generating a prediction model of protein titer and/or product quality attributes based on said correlation(s); and
      (iii) storing said prediction model in a database.
145. The system of Enumerated Example 144, wherein said operations further comprise:
   (iv) determining one or more settings for said one or more vessels using said prediction model; and
   (v) storing said determined setting(s), said data inputs from said one or more analyzers and said data inputs from said one or more detectors in said database.
146. The system of Enumerated Example 144 or Enumerated Example 145, wherein said data input from said one or more analyzers of said prediction model generator includes quantification of gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O2%, temperature-corrected pH, temperature-corrected partial pressure of oxygen, temperature-corrected partial pressure of carbon dioxide, O2 saturation, CO2 saturation, and/or bicarbonate.
147. The system of any one of Enumerated Examples 144-146, wherein said one or more analyzers are coupled online to said one or more ports.
148. The system of any one of Enumerated Examples 145-147, wherein said one or more settings of said prediction model generator are selected from a group consisting of pH, temperature, agitation rates, sparging rates, nutrient feeds, fluid control valves, and/or batch duration.
149. The system of any one of Enumerated Examples 144-148, wherein said operations of said prediction model generator further comprise comparing said protein titer and/or said product quality attributes to a performance target, wherein said performance target is based on values set forth in good manufacturing practices (GMP) or regulatory standards.
150. The system of any one of Enumerated Examples 138-149, further comprising a manual override disabling said signal generator.
151. The system of any one of Enumerated Examples 138-150, further comprising a report generator for generating a report from said prediction model.
152. The system of any one of Enumerated Examples 140-151, wherein said fluid control valves control transfer of said cells from one vessel to another vessel.
153. The system of any one of Enumerated Examples 138-152, wherein said prediction model comprises a random forest model, a batch evolution model and/or a batch level model.
154. The system of any one of Enumerated Examples 138-153, wherein said recombinant protein is dupilumab.
155. The system of any one of Enumerated Examples 59-75, wherein said recombinant protein is dupilumab.
156. A method for controlling the duration of a cell culture batch, comprising:
   (a) culturing cells in a cell culture, wherein said cells produce a recombinant protein and said cell culture undergoes at least one dissolved oxygen (DO) excursion;
   (b) correlating at least one cell culture health parameter to at least one predicted product quality using at least one regression model; and
   (c) controlling the duration of said cell culture batch based on said at least one predicted product quality.
157. The method of Enumerated Example 156, wherein said controlling further comprises comparing each of said at least one predicted product qualities to a performance target for said product quality.
158. The method of Enumerated Example 156 or Enumerated Example 157, wherein said controlling further comprises terminating said cell culture batch when said at least one predicted product quality fails to meet said performance target.
159. The method of any one of Enumerated Examples 156-158, wherein said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.
160. The method of Enumerated Example 159, wherein said at least one product quality comprises protein titer and a performance target for protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.
161. The method of Enumerated Example 159 or Enumerated Example 160, wherein said at least one product quality comprises protein purity and a performance target for protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%.
162. The method of Enumerated Example 161, wherein said protein purity is measured by liquid chromatography, capillary electrophoresis, gel electrophoresis, or mass spectrometry.
163. The method of any one of Enumerated Examples 159-162, wherein said at least one product quality comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
164. The method of Enumerated Example 163, wherein said high molecular weight species are measured by liquid chromatography, capillary electrophoresis or gel electrophoresis.
165. The method of any one of Enumerated Examples 159-164, wherein said at least one product quality comprises low molecular weight species and a performance target for said lower molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
166. The method of Enumerated Example 165, wherein said low molecular weight species are measured by liquid chromatography, capillary electrophoresis or gel electrophoresis.
167. The method of any one of Enumerated Examples 159-166, wherein said at least one product quality comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%.
168. The method of Enumerated Example 167, wherein said non-glycosylated heavy chain is measured by reduced capillary electrophoresis.
169. The method of any one of Enumerated Examples 159-168, wherein said at least one product quality comprises charge variant profile and a performance target for said charge variant profile is an upper limit for a charge variant of about 50%, about 45%, about 40%, about 35%, about 30%, about 25%, about 20%, or about 15%.
170. The method of Enumerated Example 169, wherein said charge variant profile is measured by imaged capillary isoelectric focusing.
171. The method of any one of Enumerated Examples 159-170, wherein said at least one product quality comprises bispecific purity and a performance target for said bispecific purity is a lower limit of about 90%, about 91%, about 92%, about 93%, about 94%, about 95%, about 96%, about 97%, about 98%, or about 99%.
172. The method of Enumerated Example 171, wherein said bispecific purity is measured by protein A chromatography or mixed mode chromatography.
173. The method of any one of Enumerated Examples 159-172, wherein said at least one product quality comprises binding impurity and a performance target for said binding impurity is an upper limit of about 10%, about 9%, about 8%, about 7%, about 6%, about 5%, about 4%, about 3%, about 2%, or about 1%.
174. The method of Enumerated Example 173, wherein said binding impurity is measured by protein A chromatography or mixed mode chromatography.
175. The method of any one of Enumerated Examples 159-174, wherein said at least one product quality comprises non-binding impurity and a performance target for said non-binding impurity is an upper limit of about 10%, about 9%, about 8%, about 7%, about 6%, about 5%, about 4%, about 3%, about 2%, or about 1%.
176. The method of Enumerated Example 175, wherein said non-binding impurity is measured by protein A chromatography or mixed mode chromatography.
177. The method of any one of Enumerated Examples 156-176, wherein said cells are selected from the group consisting of CHO cells, HEK293 cells, and BHK cells.
178. The method of Enumerated Example 177, wherein said CHO cells are selected from the group consisting of CHO-K1, CHO DUX B-11, Veggie-CHO, GS-CHO, S-CHO, or CHO lec.
179. The method of any one of Enumerated Examples 156-178, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.
180. The method of any one of Enumerated Examples 156-179, wherein said recombinant protein is dupilumab.
181. The method of any one of Enumerated Examples 156-180, wherein said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.
182. The method of any one of Enumerated Examples 156-181, wherein said at least one DO excursion has a duration of less than 360 hours, from 240 hours to 360 hours, from 24 hours to 260 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.
183. The method of any one of Enumerated Examples 156-182, wherein said at least one DO excursion occurs during an early phase, a middle phase, and/or a late phase of said cell culture.
184. The method of any one of Enumerated Examples 156-183, wherein said at least one regression model comprises a random forest model, a batch evolution model and/or a batch level model.
185. The method of any one of Enumerated Examples 156-184, wherein said at least one cell culture health parameter comprises one or more of pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O2%, temperature-corrected pH, temperature-corrected partial pressure of oxygen, temperature-corrected partial pressure of carbon dioxide, O2 saturation, CO2 saturation, and/or bicarbonate.
186. A method for modifying a cell culture process, comprising:
   (a) culturing cells in a cell culture according to a first cell culture process, wherein said cells produce a recombinant protein and said cell culture undergoes at least one DO excursion;
   (b) predicting at least one recombinant protein parameter by using at least one regression model correlating at least one cell culture health parameter to said at least one recombinant protein parameter; and
   (c) culturing said cells according to a second cell culture process, wherein said second cell culture process is modified from said first cell culture process using said at least one predicted recombinant protein parameter.
187. The method of Enumerated Example 186, wherein modifying said first cell culture process comprises modifying a feed schedule, modifying a feed quantity, modifying an agitation rate, modifying a sparging rate, modifying a DO setpoint, and/or modifying a cell culture batch duration.
188. The method of Enumerated Example 186 or Enumerated Example 187, further comprising comparing each of said at least one recombinant protein parameters to a performance target for said recombinant protein parameter.
189. The method of any one of Enumerated Examples 186-188, wherein said at least one recombinant protein parameter is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.
190. The method of Enumerated Example 189, wherein said at least one recombinant protein parameter comprises protein titer and a performance target for said protein titer is at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, or at least 12 g/L.
191. The method of Enumerated Example 189 or Enumerated Example 190, wherein said at least one recombinant protein parameter comprises protein purity and a performance target for said protein purity is a lower limit of about 80%, about 85%, about 86%, about 87%, about 88%, about 89%, about 90%, about 91%, about 92%, about 93%, about 94%, about 95%, about 96%, about 97%, about 98%, or about 99%.
192. The method of Enumerated Example 191, wherein said protein purity is measured by liquid chromatography, capillary electrophoresis, gel electrophoresis, or mass spectrometry.
193. The method of any one of Enumerated Examples 189-192, wherein said at least one recombinant protein parameter comprises high molecular weight species and a performance target for said high molecular weight species is an upper limit of about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
194. The method of Enumerated Example 193, wherein said high molecular weight species are measured by liquid chromatography, capillary electrophoresis or gel electrophoresis.
195. The method of any one of Enumerated Examples 189-194, wherein said at least one recombinant protein parameter comprises low molecular weight species and a performance target for said low molecular weight species is an upper limit of about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1%.
196. The method of Enumerated Example 195, wherein said low molecular weight species are measured by liquid chromatography, capillary electrophoresis or gel electrophoresis.
197. The method of any one of Enumerated Examples 189-196, wherein said at least one recombinant protein parameter comprises non-glycosylated heavy chain and a performance target for said non-glycosylated heavy chain is an upper limit of about 10%, about 9%, about 8%, about 7%, about 6%, about 5%, about 4%, about 3%, about 2%, or about 1%.
198. The method of Enumerated Example 197, wherein said non-glycosylated heavy chain is measured by reduced capillary electrophoresis.
199. The method of any one of Enumerated Examples 189-198, wherein said at least one recombinant protein parameter comprises charge variant profile and a performance target for said recombinant protein parameter is an upper limit for a charge variant of about 50%, about 45%, about 40%, about 35%, about 30%, about 25%, about 20%, or about 15%.
200. The method of Enumerated Example 199, wherein said charge variant profile is measured by imaged capillary isoelectric focusing.
201. The method of any one of Enumerated Examples 189-200, wherein said at least one recombinant protein parameter comprises bispecific purity and a performance target for said bispecific purity is a lower limit of about 90%, about 91%, about 92%, about 93%, about 94%, about 95%, about 96%, about 97%, about 98%, or about 99%.
202. The method of Enumerated Example 201, wherein said bispecific purity is measured by protein A chromatography or mixed mode chromatography.
203. The method of any one of Enumerated Examples 189-202, wherein said at least one recombinant protein parameter comprises binding impurity and a performance target for said binding impurity is an upper limit of about 10%, about 9%, about 8%, about 7%, about 6%, about 5%, about 4%, about 3%, about 2%, or about 1%.
204. The method of Enumerated Example 203, wherein said binding impurity is measured by protein A chromatography or mixed mode chromatography.
205. The method of any one of Enumerated Examples 189-204, wherein said at least one recombinant protein parameter comprises non-binding impurity and a performance target for said non-binding impurity is an upper limit of about 10%, about 9%, about 8%, about 7%, about 6%, about 5%, about 4%, about 3%, about 2%, or about 1%.
206. The method of Enumerated Example 205, wherein said non-binding impurity is measured by protein A chromatography or mixed mode chromatography.
207. The method of any one of Enumerated Examples 186-206, wherein said cells are selected from the group consisting of CHO cells, HEK293 cells, and BHK cells.
208. The method of Enumerated Example 207, wherein said CHO cells are selected from the group consisting of CHO-K1, CHO DUX B-11, Veggie-CHO, GS-CHO, S-CHO, or CHO lec.
209. The method of any one of Enumerated Examples 186-208, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.
210. The method of any one of Enumerated Examples 186-209, wherein said recombinant protein is dupilumab.
211. The method of any one of Enumerated Examples 186-210, wherein said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%,
212. The method of any one of Enumerated Examples 186-211, wherein said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.
213. The method of any one of Enumerated Examples 186-212, wherein said at least one DO excursion occurs during an early phase, a middle phase, and/or a late phase of said cell culture.
214. The method of any one of Enumerated Examples 186-213, wherein said at least one regression model comprises a random forest model, a batch evolution model and/or a batch level model.
215. The method of any one of Enumerated Examples 186-214, wherein said at least one cell culture health parameter comprises one or more of pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O2%, temperature-corrected pH, temperature-corrected partial pressure of oxygen, temperature-corrected partial pressure of carbon dioxide, O2 saturation, CO2 saturation, and/or bicarbonate.
216. A method for predicting at least one property of a cell culture batch, comprising:
   (a) measuring at least one condition of a cell culture batch, wherein said cells produce a recombinant protein and said cell culture batch undergoes at least one DO excursion; and
   (b) predicting at least one property of said cell culture batch by using at least one regression model correlating said at least one condition to said at least one property.
217. The method of Enumerated Example 216, wherein said at least one condition is one or more of DO excursion magnitude, DO excursion duration, DO excursion timing, gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O2%, temperature-corrected pH, temperature-corrected partial pressure of oxygen, temperature-corrected partial pressure of carbon dioxide, O2 saturation, CO2 saturation, and bicarbonate.
218. The method of Enumerated Example 216 or Enumerated Example 217, wherein said at least one property is one or more of viable cell density, viability, protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and terminal galactosylation.
219. The method of any one of Enumerated Examples 216-218, further comprising:
   (c) modifying a cell culture process for said cell culture batch using said prediction of said at least one property.
220. The method of Enumerated Example 219, wherein modifying said cell culture process comprises modifying a feed schedule, modifying a feed quantity, modifying an agitation rate, modifying a sparging rate, modifying a DO setpoint, and/or modifying a cell culture batch duration.
221. The method of any one of Enumerated Examples 216-220, wherein said recombinant protein is dupilumab.
222. A method for improving dupilumab production, comprising:
   (a) culturing cells in a cell culture according to a first cell culture process, wherein said cells produce dupilumab and said cell culture undergoes at least one DO excursion;
   (b) predicting at least one dupilumab product quality by using at least one regression model correlating at least one cell culture condition to said at least one product quality; and
   (c) culturing said cells according to a second cell culture process, wherein said second cell culture process is modified from said first cell culture process using said at least one predicted product quality to improve dupilumab production.
223. The method of Enumerated Example 222, wherein said at least one cell culture condition is one or more of DO excursion magnitude, DO excursion duration, DO excursion timing, gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O2%, temperature-corrected pH, temperature-corrected partial pressure of oxygen, temperature-corrected partial pressure of carbon dioxide, O2 saturation, CO2 saturation, and bicarbonate.
224. The method of Enumerated Example 222 or Enumerated Example 223, wherein modifying said first cell culture process comprises modifying a feed schedule, modifying a feed quantity, modifying an agitation rate, modifying a sparging rate, modifying a DO setpoint, and/or modifying a cell culture batch duration.
225. The method of any one of Enumerated Examples 222-224, further comprising comparing each of said at least one product quality to a performance target for said product quality.
226. The method of any one of Enumerated Examples 222-225, wherein said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.
227. The method of any one of Enumerated Examples 222-226, wherein said cells are selected from the group consisting of CHO-K1, CHO DUX B-11, Veggie-CHO, GS-CHO, S-CHO, or CHO lec.
228. The method of any one of Enumerated Examples 222-227, wherein said at least one DO excursion includes a DO of from 0% to 40%, from 0% to 20%, from 0% to 8%, about 0%, about 1%, about 2%, about 3%, about 4%, about 5%, about 6%, about 7%, about 8%, about 9%, or about 10%.
229. The method of any one of Enumerated Examples 222-228, wherein said at least one DO excursion has a duration of less than 360 hours, from 240 hours to 360 hours, from 24 hours to 360 hours, less than 300 hours, from 1 hour to 24 hours, from 4 hours to 48 hours, from 24 hours to 288 hours, about 1 hour, about 2 hours, about 3 hours, about 4 hours, about 5 hours, about 6 hours, about 10 hours, about 12 hours, about 18 hours, about 20 hours, about 24 hours, about 36 hours, about 48 hours, about 60 hours, about 72 hours, about 84 hours, about 96 hours, about 108 hours, about 120 hours, about 132 hours, about 144 hours, about 168 hours, about 192 hours, about 216 hours, about 240 hours, about 264 hours, about 288 hours, about 312 hours, about 336 hours, or about 360 hours.
230. The method of any one of Enumerated Examples 222-229, wherein said at least one DO excursion occurs during an early phase, a middle phase, and/or a late phase of said cell culture.
231. The method of any one of Enumerated Examples 222-230, wherein said at least one regression model comprises a random forest model, a batch evolution model and/or a batch level model.
232. The method of any one of Enumerated Examples 1-29, 76-106, 156-185, and 222-231, wherein said at least one product quality is a product quality attribute.
233. The method of any one of Enumerated Examples 30-58, 107-137, and 186-215, wherein said at least one recombinant protein parameter is a product quality attribute.
234. The method of any one of Enumerated Examples 216-221, wherein said at least one property is a product quality attribute.

What is claimed is:

1. A method for controlling the duration of a cell culture batch, comprising:
(a) culturing cells in a cell culture, wherein said cells produce a recombinant protein and said cell culture undergoes at least one dissolved oxygen (DO) excursion;
(b) correlating said at least one DO excursion to at least one predicted product quality using at least one regression model; and
(c) controlling the duration of said cell culture batch based on said at least one predicted product quality.

2. The method of claim 1, wherein said controlling further comprises comparing each of said at least one predicted product qualities to a performance target for said product quality.

3. The method of claim 1, wherein said controlling further comprises terminating said cell culture batch when said at least one predicted product quality fails to meet said performance target.

4. The method of claim 1, wherein said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.

5. The method of claim 1, wherein said cells are selected from the group consisting of CHO cells, HEK293 cells, and BHK cells.

6. The method of claim 1, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.

7. A method for controlling the duration of a cell culture batch, comprising:
(a) culturing cells in a cell culture, wherein said cells produce a recombinant protein and said cell culture undergoes at least one dissolved oxygen (DO) excursion;
(b) correlating the expression of at least one gene in said cells to at least one predicted product quality using at least one regression model; and
(c) controlling the duration of said cell culture batch based on said at least one predicted product quality.

8. The method of claim 7, wherein said controlling further comprises comparing each of said at least one predicted product qualities to a performance target for said product quality.

9. The method of claim 7, wherein said controlling further comprises terminating said cell culture batch when said at least one predicted product quality fails to meet said performance target.

10. The method of claim 7, wherein said at least one product quality is selected from the group consisting of protein titer, protein purity, low molecular weight (LMW) species, high molecular weight (HMW) species, non-glycosylated heavy chain, charge variant profile, glycosylation profile, bispecific purity, binding impurity, non-binding impurity, oxidized methionine, glycation, deamidation, trisulfide, disulfide shuffling, N-terminal pyroglutamate, amino acid sequence variants, C-terminal lysine removal, proteolysis, and/or terminal galactosylation.

11. The method of claim 7, wherein said recombinant protein is selected from a group consisting of an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, a variant thereof, a fragment thereof, and/or a multimer thereof.

12. The method of claim 7, wherein said at least one regression model comprises a random forest model, a batch evolution model and/or a batch level model.

13. The method of claim 7, wherein the expression of said at least one gene is measured using RNA sequencing (RNAseq), quantitative polymerase chain reaction (qPCR), or a microarray.

14. The method of claim 7, wherein said at least one gene is a hypoxia-related gene.

15. A system for improved production of a recombinant protein, said system comprising:
(a) one or more vessels for culturing cells expressing a recombinant protein;
(b) one or more ports for collecting one or more samples from said one or more vessels;
(c) one or more analyzers for analyzing said one or more samples;
(d) one or more modules for controlling one or more settings in said one or more vessels;
(e) a computing system coupled to said one or more analyzers and said one or more modules;
(f) a prediction model stored in non-transitory machine-readable medium coupled to said computing system; and
(g) a signal generator for controlling said one or more modules.

16. The system of claim 15, wherein said one or more analyzers are configured to analyze gene expression, pH, partial pressure of oxygen, partial pressure of carbon dioxide, glutamine, glutamate, glucose, lactate, ammonium, sodium, potassium, calcium, phosphate, IgG, osmolality, total density, viable density, viability, average live diameter, vessel temperature, vessel pressure, sparging O₂%, temperature-corrected pH, temperature-corrected partial pressure of oxygen, temperature-corrected partial pressure of carbon dioxide, O₂ saturation, CO₂ saturation, and/or bicarbonate.

17. The system of claim 15, wherein said one or more analyzers are coupled online to said one or more ports.

18. The system of claim 15, wherein said one or more settings are selected from a group consisting of pH, temperature, agitation rates, sparging rates, nutrient feeds, fluid control valves, and/or batch duration.

19. The system of claim 15, further comprising one or more detectors for characterizing protein titer and/or product quality attributes.

20. The system of claim 19, wherein said characterizing comprises imaged capillary isoelectric focusing (iCIEF), capillary electrophoresis (CE), microfluidic capillary electrophoresis (MCE), size exclusion chromatography (SEC), size exclusion-ultra-performance liquid chromatography (SE-UPLC), high-performance liquid chromatography (HPLC), mass spectrometry (MS), tandem mass spectrometry (MS/MS), liquid chromatography-mass spectrometry (LC-MS), capillary electrophoresis-mass spectrometry (CE-MS), gel electrophoresis, nuclear magnetic resonance, and/or surface plasmon resonance.
